(19) United States
(12) Patent Application Publication
(10) Pub. No.: US 2026/0215385 A1
(43) Pub. Date: Jul. 30, 2026

(54) FUSARIUM RESISTANT LETTUCE PLANTS

(71) Applicant: NUNHEMS B.V., Nunhem (NL)

(72) Inventors: Gowtham PRAKASH, Nunhem (NL); Daan SCHREURS, Nunhem (NL); Robert Johannes Martinus RAEDTS, Nunhem (NL); Michiel VAN DEN MUNCKHOF, Nunhem (NL); Bart GERAATS, Nunhem (NL); Annick HOOGENDAM, Nunhem (NL); Noor BREUGELMANS-DROUEN, Nunhem (NL)

(21) Appl. No.: 19/159,020

(22) PCT Filed: Feb. 20, 2024
(86) PCT No.: PCT/EP2024/054255

§ 371 Date: Aug. 22, 2025

(30) Foreign Application Priority Data
Feb. 23, 2023 (EP) 23158335.2

(51) Int. Cl. A01H 6/14; A01H 1/00; A01H 1/02; A01H 1/04; A01H 5/12

(52) U.S. Cl. CPC A01H 6/1472; A01H 1/021; A01H 1/045; A01H 1/1255; A01H 5/12

(57) ABSTRACT

The invention relates to a lettuce plant (Lactuca sativa L.) comprising a resistance QTL which confers dual resistance to Fusarium oxysporum f.sp lactucae race Fol-1 and race Fol-4, wherein the QTL is located on chromosome 7. The invention further provides seeds, progeny, propagation material and food products from the plant.

### FIELD OF THE INVENTION

The present invention relates to plants and plant parts, in particular lettuce plants (Lactuca sativa L.), which have a single Quantitative Trait locus (QTL7.1) from Lactuca serriola conferring resistance to Fusarium oxysporum f.sp. lactucae (Fol) race 1 (Fol-1) and/or race 4 (Fol-4), especially conferring dual resistance to Fol-1 and Fol-4. The invention further relates to parts of these plants, to seeds, to propagation material, to the progeny of these plants, and use of the plants as germplasm in breeding for at least Fol-1 and/or Fol-4 resistance, or for breeding for dual Fol-1 and Fol-4 resistance. The QTL7.1 was identified in an accession of the wild species Lactuca serriola and was introgressed into Lactuca sativa.

### BACKGROUND OF THE INVENTION

A common disease of lettuce worldwide is Fusarium wilt, which was first observed in Japan in 1955. Later Fusarium wilt has been identified in the United States, Taiwan, Iran, Portugal, Italy, and Brazil. Initially three races (Fol-1, Fol-2, Fol-3) of pathogenic Fusarium oxysporum f. sp. lactucae have been identified in lettuce, until a fourth race, Fol-4, was identified in 2017 in the Netherlands (Gilardi et al. 2017, A new race of Fusarium oxysporum f. sp. lactucae of lettuce. Plant Pathol 66:677-688). Gilardi et al. refers to the race as ‘race 4’ and later the ISF adopted this race as new race Fol: 4, taking the race 04750888 of Gilard et al. as representative of type Fol: 4 see (world wide web at//worldseed.org/document/differential-sets-fol-race-4/).

The four races, referred to as Fol-1, Fol-2, Fol-3 and Fol-4 herein (or as Fol: 1, Fol: 2, Fol: 3 and Fol: 4 by the ISF), can be differentiated based on e.g. the phenotypes on differential lettuce hosts, as published by the ISF (world wide web at//worldseed.org/document/differential-sets-fol-race-4/) and based on e.g. race-specific PCR. To differentiate the resistance reactions of the races on the host varieties, the CPVO protocol described on the world wide web at cpvo.europa.eu is used.

Race Fol-4 is increasingly spreading. Claerbout et al. (2022,//doi.org/10.1111/ppa.13668) reported that in Belgium 91% of Fusarium isolates found in commercial lettuce greenhouses belonged to race Fol-4, while only 6% belonged to race Fol-1. Also, variety Patriot was found to be a good variety for differentiating Fol-4.

Two QTLs conferring resistance against race Fol-1 were identified in crisphead lettuce cultivar (L. sativa) VI185 and were mapped to chromosome 7 (qFOL7.1) and chromosome 8 (qFOL8.1) by Seki et al. (2020, Euphytica 216:174). qFOL7.1 was mapped to chromosome 7 of the V8 genome at 85.69 Mb-100.59 Mb (shoot) and at 87.943 Mb-98.36 Mb (root), see Table 2 therein. In contrast, the present QTL7.1 is located at about 61.49 Mb-66.46 Mb of the V8 genome (data not shown).

Also in Spain the occurrence of race Fol-4 has recently been reported, as confirmed by race-specific PCR and pathogenicity tests (Plant Dis. 2023 Feb. 12. doi: 10.1094/PDIS-12-22-2819-PDN; Online ahead of print).

US2022/0346339 describes a lettuce variety called NUN06240 as comprising resistance to race 4 and to race 1. Applicant has compared the chromosome 7 SNP haplotype of NUN06240 with the SNP donor haplotype of the QTL7.1 and found that these are significantly different. Further Applicant has confirmed that in NUN06240 two separate loci are responsible for the resistance against race 1 and against race 4 and these two loci are not on chromosome 7. Therefore NUN06240 does not contain the instant QTL7.1.

Garibaldi et al. 2004 (Crop Protection 23:845-851) describe “one Italian isolate (FOL 4)” in pathogenicity tests. On page 850 (Left column, Discussion) it is mentioned that this isolate is a ‘race 1’ isolate, i.e. what is herein referred to as Fol-1. Fol-4 did not exist at the time of the publication and the name was likely just given by numbering isolates of the collection rather and not by reference to the race.

Murray et al. (2021, HortScience 56 (12): 1552-1564) describe a study to identify resistance in Florida, USA, against race Fol-1, which was detected in Florida for the first time in 2017. Fol-4 was not present in the USA at that time. Murray et al. mainly screened Florida adapted Lactuca sativa cultivars for resistance against Fol-1 but also included some wild Lactuca accessions. However, no resistance to root symptoms of Fol-1 was identified amongst the wild Lactuca accessions tested (page 1562, right column, lines 1-3).

Mestdagh et al. (2023, Front. Plant Sci., Volume 14-2023,//doi.org/10.3389/fpls.2023.1272136) have developed a PCR assay to differentiate between race Fol-1 and race Fol-4.

As race Fol-4 is spreading rapidly, there is an urgent need to develop lettuce plants that are resistant against race Fol-4. No such resistance has been identified yet.

### FIGURES

The present invention will be further elucidated in the examples that follow. These examples are for illustrative purposes only and are not to be construed as limiting the invention in any way. The examples make reference to the following figures:

FIG. 1: Foto taken 12 days after inoculation with Fol-4; plants of the L. sativa line comprising QTL7.1 are shown in the black square, showing high resistance to race Fol-4.

FIG. 2: Foto taken 12 days after inoculation with Fol-1; plants of the L. sativa line comprising QTL7.1 are shown in the black square, showing intermediate resistance to race Fol-1.

FIG. 3: Blast output of SEQ ID NO: 1 (Query) against L. sativa V11 genome (Subject); SNP_01 is at nucleotide 64382881 of chromosome 7 and is a G (Guanine) in the L. sativa reference genome V11.

FIG. 4: Schematic diagram of chromosome 7 and the position of QTL7.1, with the QTL7.1 flanking markers (SNP_01 and SNP_10) and the QTL7.1 peak marker (not according to scale); the peak marker is SNP_07 and markers SNP_01 to SNP_06 are ‘upstream’ of SNP_07 on the chromosome, while markers SNP_08 to SNP_10 are ‘downstream’ of SNP_07 on the chromosome.

### GENERAL DEFINITIONS

The indefinite article “a” or “an” does not exclude the possibility that more than one of the element is present, unless the context clearly requires that there be one and only one of the elements. The indefinite article “a” or “an” thus usually means “at least one”.

As used herein, the term “plant” includes the whole plant or any parts or derivatives thereof, such as plant organs (e.g., harvested or non-harvested leaves, seeds, etc.), plant cells, plant protoplasts, plant cell or tissue cultures from which whole plants can be regenerated, plant calli, plant cell clumps, parts of plants, such as embryos, pollen, ovules, ovaries (e.g. harvested tissues or organs, such as harvested heads or parts thereof), flowers, leaves, seeds, clonally propagated plants, roots, stems, root tips and the like. Also any developmental stage is included, such as seedlings, immature and mature, etc. When “seeds of a plant” are referred to, these either refer to seeds from which the plant can be grown or to seeds produced on the plant, after self-fertilization or cross-fertilization.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of one or a few loci or genes (or phenotypic characteristics due to these specific loci or genes), but which can otherwise differ from one another enormously as regards the other loci or genes. Thus, e.g. a plant defined only by the presence of one QTL is not a plant variety, as thousands of other genes which define a plant variety are undefined and a plant defined only by the presence of QTL7.1 is not uniform and stable for these thousands of genes and the characteristics conferred by these genes. QTL7.1 can be used to develop many different lettuce varieties, e.g. a lettuce variety which is uniform and stable for its physiological and morphological characteristics such as leaf size or shape, leaf margins and color, etc. and which also comprises QTL7.1.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous). Thus, for example reference may herein be made to a “Fol-4 and/or Fol-1 resistance allele” of QTL7.1.

The term “gene” means a (genomic) DNA sequence comprising a region (transcribed region), which is transcribed into a messenger RNA molecule (mRNA) in a cell, and an operably linked regulatory region (e.g. a promoter). Different alleles of a gene are thus different alternatives form of the gene, which may be in the form of e.g. differences in one or more nucleotides of the genomic DNA sequence (e.g. in the promoter sequence, the exon sequences, intron sequences, etc.), mRNA and/or amino acid sequence of the encoded protein.

The term “locus” (loci plural) means a specific place or places or a site on a chromosome where for example a QTL, a gene or genetic marker is found. The Fol-4 and/or Fol-1 resistance locus is, thus, the location in the genome of L. sativa, where QTL7.1 is found.

A “quantitative trait locus”, or “QTL” is a chromosomal locus that encodes for one or more alleles that affect the expressivity of a continuously distributed (quantitative) phenotype. The Fol-4 and/or Fol-1 resistance conferring quantitative trait locus is named QTL7.1 herein.

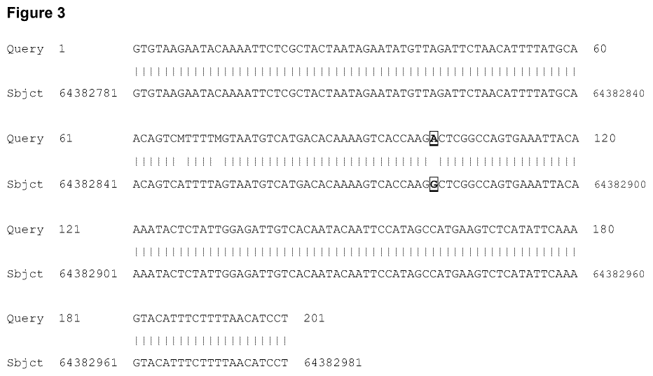

“Lactuca sativa genome” and “physical position on the lettuce genome” and “chromosome 7” refers to the physical genome of the reference genome V11, and the physical chromosomes and the physical position on the chromosomes. So, for example SNP_01 is located at the nucleotide (or ‘base’) positioned physically at nucleotide 64.382.881 of chromosome 7, as shown in FIG. 3.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). In lettuce, wild relatives of cultivated lettuce can be used to introgress fragments of the wild genome into the genome of cultivated Lactuca sativa genome L. Such a cultivated lettuce plant thus has a “genome of cultivated Lactuca sativa” but comprises in the genome a fragment on chromosome 7 of a wild L. serriola donor. So, for example, a cultivated lettuce plant is provided herein comprising a genome of L. sativa, and in that genome one introgression fragment on chromosome 7 which confers Fol-4 and/or Fol-1 resistance compared to the cultivated L. sativa genome lacking the introgression fragment (and having a chromosomes 7 of cultivated L. sativa without the introgression fragment). It is understood that the term “introgression fragment” never includes a whole chromosome, but only a part of a chromosome. The introgression fragment can be large, e.g. even three quarter or half of a chromosome, but is preferably smaller, such as about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

“Lettuce” or “cultivated lettuce” or “cultivated Lactuca sativa” refers herein to plants of the species Lactuca sativa L. (or seeds from which the plants can be grown), and parts of such plants, bred by humans for food and having good agronomic characteristics. This includes any cultivated lettuce, such as breeding lines (e.g. backcross lines, inbred lines), cultivars and varieties of any type.

Generally heading and non-heading types of lettuce are distinguished. Heading types include for example crisphead, butterhead and romaine (cos) types, while non-heading types include leaf-types. Cultivated lettuce plants are not “wild lettuce” plants or “wild Lactuca” plants, i.e. plants which generally have much poorer yields and poorer agronomic characteristics than cultivated plants and e.g. grow naturally in wild populations.

“Wild lettuce” or “wild Lactuca” accessions refers to plants of species other than cultivated Lactuca sativa, such as Lactuca virosa, Lactuca serriola, Lactuca saligna, Lactuca perennis, and others. Preferably, such wild lettuce comprises or consists of Lactuca species which are cross fertile with L. sativa, optionally with the aid of embryo rescue techniques (see Maisonneuve 1987, Agronomique 7:313-319 and Maisonneuve et al. 1995, Euphytica 85:281-285) and/or chromosome doubling techniques (Thompson and Ryder 1961, US Dept Agric Tech Bul. 1224), or methods whereby genes can be transferred into L. sativa via a bridge species, such as L. serriola.

“Somatic cells” and “reproductive cells” can be distinguished, whereby somatic cells are cells other than gametes (e.g. ovules and pollen), germ cells and gametocytes. Gametes, germ cells and gametocytes are “reproductive cells”.

“Tissue Culture” or “cell culture” refers to an in vitro composition comprising isolated cells of the same or a different type or a collection of such cells organized into plant tissue. Tissue cultures and cell cultures of lettuce, and regeneration of lettuce plants therefrom, is well known and widely published (see, e.g., Teng et al., HortScience. 1992, 27 (9): 1030-1032 Teng et al., HortScience. 1993, 28 (6): 669-1671, Zhang et al., Journal of Genetics and Breeding. 1992, 46 (3): 287-290).

“Harvested plant material” refers herein to plant parts (e.g., leaves, leaf parts or heads detached from the whole plant) which have been collected for further storage and/or further use.

“Harvested seeds” refers to seeds harvested from a line or variety, e.g., produced after self-fertilization or cross-fertilization and collected.

“Harvested leaves” or “harvested heads” as used herein refers to lettuce leaves, or leaf parts or heads, i.e., the plant without the root system, for example substantially all (harvested) leaves. Leaves may be whole or cut into parts.

“Progeny” or “progenies” or “descendants” as used herein refers to offspring, or the first and all further descendants derived from (obtained from) (derivable from or obtainable from) a plant of the invention that comprises (retains) the QTL7.1 according to the invention. Progeny may be derived by regeneration of cell culture or tissue culture, or parts of a plant, or selfing of a plant, or by producing seeds of a plant. In further embodiments, progeny may also encompass lettuce plants derived from crossing of at least one lettuce plant with another lettuce plant of the same or another variety or (breeding) line, or wild Lactuca plants, backcrossing, inserting of a locus into a plant or mutation. A progeny is, e.g., a first generation progeny, i.e. the progeny is directly derived from, obtained from, obtainable from or derivable from the parent plant by, e.g., traditional breeding methods (selfing and/or crossing) or regeneration or transformation. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above. Also double haploid plants are progeny.

A “plant line” or “breeding line” refers to a plant and its progeny being highly uniform in plant phenotype. As used herein, the term “inbred line” refers to a plant line which has been repeatedly selfed and is nearly homozygous for all alleles. Thus, an “inbred line” or “parent line” refers to a plant which has undergone several generations (e.g. at least 2, 3, 4, 5, 6, 7 or more) of inbreeding, resulting in a plant line with a high uniformity.

“F1, F2, F3, etc.” refers to the consecutive related generations following a cross between two parent plants or parent lines. The plants grown from the seeds produced by crossing two plants or lines is called the F1 generation. Selfing the F1 plants results in the F2 generation, etc.

“Hybrid” refers to the seeds harvested from crossing one plant line or variety with another plant line or variety, and the plants or plant parts grown from said seeds.

“F1 hybrid” plant (or F1 hybrid seed) is the generation obtained from crossing two non-isogenic inbred parent lines. Thus, F1 hybrid seeds are seeds from which F1 hybrid plants grow.

An “interspecific hybrid” refers to a hybrid produced from crossing a plant of one species, e.g. L. sativa, with a plant of another species, e.g. L. serriola.

“Crossing” refers to the mating of two parent plants. Equally “Cross-pollination” refers to fertilization by the union of two gametes from different plants.

“Selfing” refers to the self-pollination of a plant, i.e. to the union of gametes from the same plant.

“Regeneration” refers to the development of a plant from cell culture or tissue culture or vegetative propagation.

“Non-propagating cell” refers to a cell which cannot be regenerated into a whole plant.

“Single (or double or triple) locus converted (conversion) plant” refers to plants which are developed by plant breeding techniques comprising or consisting of backcrossing, wherein essentially all of the desired morphological and/or physiological characteristics of a lettuce plant are recovered in addition to the characteristics of the single locus (or two or three loci) having been transferred into the plant via e.g. the backcrossing technique.

“Transgene” or “chimeric gene” refers to a genetic locus comprising a DNA sequence which has been introduced into the genome of a lettuce plant by transformation. A plant comprising a transgene stably integrated into its genome is referred to as “transgenic plant”.

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimizes the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS as available on the world wide web under ebi.ac.uk/Tools/psa/emboss_needle/). Alternatively, sequence similarity or identity may be determined by searching against databases such as FASTA, BLAST, etc., but hits should be retrieved and aligned pairwise to compare sequence identity. Two proteins or two protein domains, or two nucleic acid sequences have “substantial sequence identity” if the percentage sequence identity is at least 90%, 95%, 98%, 99% or more (as determined by Emboss “needle” using default parameters, i.e. gap creation penalty=10, gap extension penalty=0.5, using scoring matrix DNAFULL for nucleic acids an Blosum62 for proteins). When reference is made to a nucleic acid sequence (e.g. DNA or genomic DNA) having “substantial sequence identity to” a reference sequence or having a sequence identity of at least 95%, 96%, 97%, 98% or 99% nucleic acid sequence identity to a reference sequence, in one embodiment said nucleotide sequence is considered substantially identical to the given nucleotide sequence and can be identified using stringent hybridisation conditions. The % identity is e.g. measured over sequences of the same length, e.g. two sequences of 201 nucleotides in length with a SNP at nucleotide 101. This can be done e.g. by pairwise alignment using e.g. Needle or by BLAST analysis of the sequence against e.g. the genomic sequence or chromosome sequence. For example a ‘SNP nucleotide at nucleotide 101 of SEQ ID NO: 1 or at nucleotide 101 in a variant sequence/or at the equivalent nucleotide in a variant sequence’, refers to the SNP nucleotide at the same position (e.g. nucleotide 101) but the flanking nucleotides to the right and to the left of the SNP may not be 100% identical to the flanking nucleotides to the left and to the right of nucleotide 101 in SEQ ID NO: 1. Both sequences of the same length, when aligned, may therefore only have 95%, 96%, 97%, 98% or 99% sequence identity.

In another embodiment, the nucleic acid sequence comprises one or more mutations compared to the given nucleotide sequence but still can be identified using stringent hybridisation conditions.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100nt) are for example those which include at least one wash in 0.2×SSC at 63° C. for 20 min, or equivalent conditions. Stringent conditions for DNA-DNA hybridisation (Southern blots using a probe of e.g. 100nt) are for example those which include at least one wash (usually 2) in 0.2×SSC at a temperature of at least 50° C., usually about 55° C., for 20 min, or equivalent conditions.

The term “Fol-4 and/or Fol-1 resistance QTL7.1” or “QTL7.1” or refers to the locus QTL7.1 introgressed into cultivated lettuce (onto cultivated L. sativa chromosome 7) from L. serriola. The term “Fol-4 and/or Fol-1 resistance QTL7.1”, thus, also encompasses QTL7.1 obtainable from the seeds deposited herein or from progeny thereof, but also from other wild L. serriola accessions. When one or two copies of the Fol-4 and/or Fol-1 resistance QTL7.1 are present at the locus in the genome (i.e. in heterozygous or homozygous form) or when two copies of the Fol-4 and/or Fol-1 resistance QTL7.1 are present (i.e. in homozygous form), the plant line or variety comprises a significantly higher resistance against races Fol-4 and/or Fol-1 than the control or genetic control lacking QTL7.1. The absence of QTL7.1 on chromosome 7 of L. sativa is herein also designated as ‘wild type’ (wt) genomic DNA of L. sativa being present at the corresponding locus.

The genotype of the SNP markers provided herein is also indicative of the absence of QTL7.1 or of QTL7.1 being present in homozygous or heterozygous form. E.g. the genotype of SNP_07 indicative of QTL7.1 is ‘TC’ or ‘TX’, with X being A, G or C (QTL7.1/wt) or ‘TT’ (QTL7.1/QTL7.1) while the genotype indicative of the wild type, i.e. of the cultivated lettuce, is ‘CC’ (wt/wt) or XX, with X being A, G or C. The genotype of SNP_06 indicative of QTL7.1 is ‘GT’ or GX with X being A, T or C (QTL7.1/wt) or ‘GG’ (QTL7.1/QTL7.1) while the genotype indicative of the wild type, i.e. of the cultivated lettuce, is ‘TT’ (wt/wt) or XX with X being A, T or C. Likewise the SNP haplotype for SNP_06 and SNP_07 of the donor is ‘G-T’.

A genetic element, an introgression fragment, or a gene or allele or QTL conferring a trait (such as Fol-4 and/or Fol-1 resistance) is said to be “obtainable from” or can be “obtained from” or “derivable from” or can be “derived from” or “as present in” or “as found in” a plant or plant part or seed or tissue or cell if it can be transferred from the plant or seed in which it is present into another plant or seed in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only seeds deposited and comprising the genetic element, locus, introgression fragment, gene or allele can be used, but also progeny/descendants from such seeds which have been selected to retain the genetic element, locus, introgression fragment, gene or allele, can be used and are encompassed herein, such as commercial varieties developed from the deposited seeds or from descendants thereof. Whether a plant (or genomic DNA, cell or tissue of a plant) comprises the same genetic element, locus, introgression fragment, gene or allele as obtainable from the deposited seeds can be determined by the skilled person using one or more techniques known in the art, such as phenotypic assays, whole genome sequencing, molecular marker analysis, trait mapping, chromosome painting, allelism tests and the like, or combinations of techniques.

A “SNP (=Single Nucleotide Polymorphism)” in context with the present invention is to be understood as a variation in a single nucleotide that occurs at a specific position in the genome. A SNP is the variation of the single nucleotide at the given position in a genome between two plants. If a wild plant having a Fol-4 and/or Fol-1 resistance (donor plant) shows in its corresponding sequence at a specific single position a nucleotide which is different from the corresponding nucleotide at the same position of a cultivated lettuce plant, the position defines a SNP between the wild donor and the cultivated lettuce. If the donor plant has one of the four possible nucleotides (A, C, T or G) at a specific position, a SNP occurs, when the cultivated plant has either of the remaining three possible nucleotides at the same corresponding sequence position. In a cultivated lettuce plant comprising an introgression fragment from a donor, it can therefore easily be determined if the single nucleotide of the SNP is from the donor or from the cultivated lettuce (recipient).

“SNP marker” refer herein to single nucleotide polymorphisms of a genomic sequence linked to QTL7.1, whereby a specific nucleotide, which is also referred to as the donor SNP nucleotide, (e.g. for SNP_07 a Thymine at nucleotide 101 of SEQ ID NO:7, or a Thymine at nucleotide 101 of a sequence comprising at least 95%, 96%, 97%, 98%, 99% sequence identity to SEQ ID NO: 7), or the sequence comprising the specific nucleotide, is linked to the QTL. This nucleotide, or sequence comprising the nucleotide, is also referred to as the ‘SNP genotype’ or ‘SNP nucleotide’ of the plant or plant part, and SNP_07 may be ‘T’ (haploid, on one chromosome) or ‘TT’ (diploid, on both chromosomes). Markers SNP_01 to SNP_10, especially SNP_02 to SNP_09 (which are in-between or flanked by SNP_01 and SNP_10), are linked to QTL7.1 and are present on the introgression fragment which comprises QTL7.1.

The ‘haplotype’ or “haploid genotype” refers to the haploid genotype of several genetic loci in a plant, especially of several SNP markers or several sequences comprising the SNP markers. For QTL7.1 the SNP haplotype may, thus, be the haploid genotype of at least 2, 3, 4, 5, 6, 7, 8, 9 or 10 SNP markers of SNP_01 to SNP_10 (or of the sequences comprising the SNP markers), or 2, 3, 4, 5, 6, 7 or 8 of SNP_02 to SNP_09 (or of the sequences comprising the SNP markers). For example, the plant comprising QTL7.1 may comprise a ‘G’ for SNP_06 at nucleotide 101 in SEQ ID NO: 6 (or at nucleotide 101 of a sequence which is at least 95%, 96%, 97%, 98% or 99% identical to SEQ ID NO: 6), a ‘T’ for SNP_07 at nucleotide 101 in SEQ ID NO: 7 (or at nucleotide 101 of a sequence which is at least 95%, 96%, 97%, 98% or 99% identical to SEQ ID NO: 7, and a ‘T’ for SNP_08 at nucleotide 101 in SEQ ID NO: 8 (or at nucleotide 101 of a sequence which is at least 95%, 96%, 97%, 98% or 99% identical to SEQ ID NO: 8), and it thus has the SNP haplotype G-T-T for SNP_06 to SNP_08, which is the SNP haplotype of SNP_06 to SNP_08 of the L. serriola donor (also referred to as the donor SNP haplotype). A diploid plant homozygous for QTL7.1 (and the introgression fragment comprising QTL7.1) would have the SNP genotype GG-TT-TT for SNP_06 to SNP_08.

A “Variant” or “orthologous” sequence or a “variant QTL7.1” refers to a Fol-4 and/or Fol-1 resistance conferring QTL7.1, or an introgression fragment comprising the QTL, which is derived from a different L. serriola donor plant than the QTL7.1 present in NCIMB44108. Such a variant QTL can e.g. be identified as having the same SNP haplotype as present in NCIMB44108 for at least 2, 3, 4, 5, 6, 7, 8, 9 or 10 markers (preferably consecutive markers) selected from SNP_01 to SNP_10, preferably selected from SNP_02 to SNP_09. So for example a plant comprising a variant QTL7.1 may comprise a SNP haplotype G-G-T-T for SNP_05 to SNP_08, i.e. a ‘G’ for SNP_05 at nucleotide 101 in SEQ ID NO: 5 (or at nucleotide 101 of a sequence which is at least 95%, 96%, 97%, 98% or 99% identical to SEQ ID NO: 5), a ‘G’ for SNP_06 at nucleotide 101 in SEQ ID NO: 6 (or at nucleotide 101 of a sequence which is at least 95%, 96%, 97%, 98% or 99% identical to SEQ ID NO: 6), a ‘T’ for SNP_07 at nucleotide 101 in SEQ ID NO: 7 (or at nucleotide 101 of a sequence which is at least 95%, 96%, 97%, 98% or 99% identical to SEQ ID NO: 7), a ‘T’ for SNP_08 at nucleotide 101 in SEQ ID NO: 8 (or at nucleotide 101 of a sequence which is at least 95%, 96%, 97%, 98% or 99% identical to SEQ ID NO: 8). In addition, the variant QTL7.1 confers confers reduced susceptibility/increased resistance to Fol-4 and/or Fol-1 infection, as described herein.

“Resistant” or “being resistant to” shall be understood in context of the present invention to mean a plant which is a host species of a particular pathogen and can, therefore, be infected by a given pathogen, but wherein the plant comprises one or more genetic element (e.g. one or more introgression fragments) resulting in reduction of pathogen growth and/or spreading in the plant after infection compared to the susceptible plant lacking the genetic elements. In context of the present invention “resistant” or “being resistant to” or in particular refers to plant cells or plants being resistant to Fusarium oxysporum f.sp. lactucae races Fol-4 and/or Fol-1, especially having at least ‘high resistance’ (HR) against race Fol-4 and/or ‘intermediate resistance’ (IR) against race Fol-1. Resistance is a relative term which can span a range of (different) reactions in the plant cell or plant, triggered by pathogen infection. The effect of those reactions by the plant cell or plant can be measured by various means. Typically, the effect is measured by defining a symptom level appearing in the plant part or plant. Typically, average symptoms (average disease score) of several plants of a line or genotype (e.g. 5, 6, 7, 8, 9, 10 or more) are compared to average symptoms (average disease score) of several plants of a control line or variety, preferably a susceptible control line or variety. Thus at least 5, 6, 7, 8, 9, 10 or more individual plants of a line or variety are scored at one or more time points post infection/inoculation and the average disease score is calculated. Concerning the present invention, the following symptom levels (or disease score) are applied according to phenotypic observations taken after Fol-4 or Fol-1 infection, using the CPVO protocol for soaking seedling roots in inoculum:
0: plant without symptoms and healthy vessels
1: plant with brown vessels only below the cotyledon, without yellowing and wilting
2: plant with brown vessels above the cotyledon, without yellowing and wilting
3: plant yellowing and wilting, brown vessels
4: dead plant

See also the Examples. The average disease score is calculated for each genotype. In one aspect a plant line or variety is said to comprise “resistance” against infection with race Fol-4 or Fol-1 if it has an average disease score of equal to or less than 2.5 for Fol-4 (e.g. equal to or less than 2.4, 2.3, 2.2, 2.1, 2.0, 1.9, 1.8, 1.7, 1.6, 1.5, 1.4, 1.3, 1.2, 1.1, 1.0, 0.9, 0.8 or 0.7) or equal to or less than 2.1 for Fol-1 (e.g. equal to or less than 2.0, 1.9, 1.8, 1.7, 1.6, 1.5, 1.4, 1.3, 1.2, 1.1, 1.0, 0.9, 0.8 or 0.7), while the susceptible control line or variety has the expected disease score, such as variety Gisela for Fol-4 or variety Patriot for Fol-1, when grown under the same conditions and infected in the same way. Preferably several susceptible control varieties are included, which need to show the expected symptoms to know that the infection has worked, see also the Differential Sets of the ISF. Resistance can be further subdivided into HR (high resistance) and IR (intermediate resistance), although for Fol-1 there is no such official subdivision. For Fol-4 the varieties Lomeria and Palmos are HR and the varieties Patriot, Rombella, Banchu Red Fire and Ballerina are IR. Gisela and Costa Rica No 4 are S (Susceptible) to Fol-4. For Fol-1, only HR (varieties Costa Rica No 4, Rombella and Palmos) or S (varieties Gisela, Patriot, Banchu Red Fire, Ballerina and Lomeria) are distinguished officially.

“Resistance against Fol-4 and/or Fol-1”, or an “increased (or enhanced) resistance against Fol-4 and/or Fol-1 infection” or a “significantly increased (or enhanced) Fol-4 and/or Fol-1 resistance” or a “reduced susceptibility to Fol-4 and/or Fol-1 infection” refers to a cultivated lettuce plant, plant line, hybrid or variety comprising QTL7.1 (or a variant thereof), having (due to QTL7.1, especially when in homozygous form) a significantly lower average disease score (on the scale of 0 to 4, described above) at one or more of the measured time-points after infection/inoculation (e.g. at 18 dpi) compared to the control plant lacking the QTL, preferably the genetic control plant or recurrent parent. Preferably the average disease score of the line or variety is decreased by at least 1.0 point, 1.5 points, 2.0 points or more on the scale of 0 to 4. In the Examples the recurrent parent plant line lacking the QTL has an average disease score of 3.6 for Fol-1 or 3.1 for Fol-4, and the introduction of QTL7.1 into the susceptible parent, especially in homozygous form, decreases the average disease score to e.g. 2.1 or less for Fol-1 and to e.g. 0.9 or less for Fol-4.

“Dual resistance” refers herein to QTL7.1 (or a variant thereof) conferring resistance against both races Fol-4 and Fol-1, e.g. IR or HR against Fol-4 and IR or HR against Fol-1.

“Control plant” is a cultivated lettuce genotype, breeding line or variety lacking QTL7.1 (or a variant thereof), e.g. lacking the introgression fragment. The control plant is preferably of the same type as the plant comprising the introgression fragment. For example, the original (e.g. susceptible) parent line into which the QTL was introgressed (also referred to as the recurrent parent) is a suitable control. Other controls are e.g. known susceptible varieties such as those of the differential set.

The term “marker assay” refers to a molecular marker assay which can be used to test whether on cultivated lettuce chromosome 7 an introgression from a L. serriola donor is present which introgression fragment comprises the QTL7.1 (or a variant thereof), by determining the genotype or haplotype of any one or more markers linked to QTL7.1, e.g. the genotype or haplotype of one or more SNP markers selected from SNP_01 to SNP_10, especially selected from SNP_02 to SNP_09.

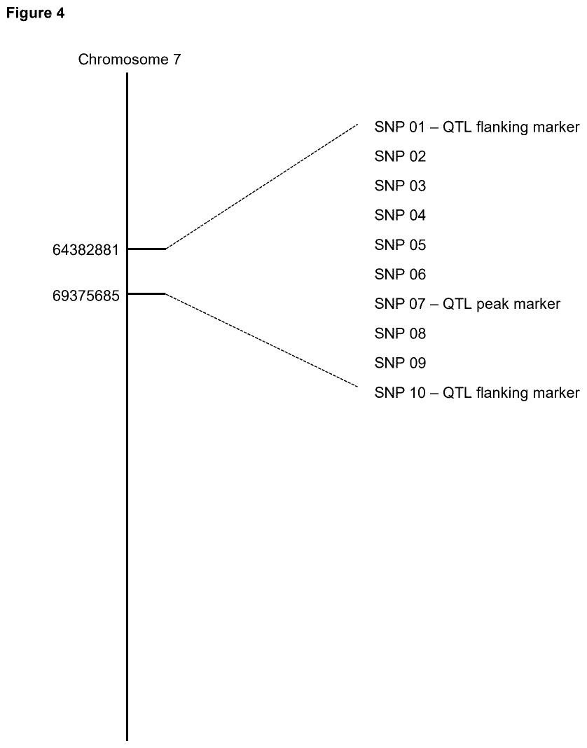

“Flanking markers” are markers which are on either side of the region comprising the QTL, i.e. the QTL is located on the chromosomal region in-between the flanking markers, e.g. the QTL7.1 (or a variant QTL7.1) is located in between the flanking markers SNP_01 and SNP_10 (see FIG. 4).

The SNP markers provided herein are located in the given order on the introgression fragment (see FIG. 4). “Consecutive” markers refers to markers in the same consecutive order, so e.g. two consecutive markers may be SNP_02 and SNP_03; SNP_03 and SNP_04, etc. and three consecutive markers may be SNP_03; SNP_02 and SNP_03 and SNP_04; etc.

“Average” or “mean” refers herein to the arithmetic mean and both terms are used interchangeably. The term “average” or “mean” thus refers to the arithmetic mean of several measurements. The skilled person understands that the phenotype of a plant line or variety depends to some extent on growing conditions and that, therefore, arithmetic means of at least 5, 6, 7, 8, 9, 10, 15, 20, 30, 40, 50 or more plants (or plant parts) are measured, preferably in randomized experimental designs with several replicates and suitable control plants grown under the same conditions in the same experiment.

“Statistically significant” or “statistically significantly” different or “significantly” different refers to a characteristic of a plant line or variety that, when compared to a suitable control (e.g. the genetic control) show a statistically significant difference in that characteristic (e.g. the p-value is less than 0.05, p<0.05, using ANOVA) from the (mean of the) control.

A “recombinant chromosome” refers to a chromosome having a new genetic makeup arising through crossing-over between homologous chromosomes, e.g. a “recombinant chromosome 7”, i.e. a chromosome 7 which is not present in either of the parent plants and arose through a rare double crossing-over event between homologous chromosomes of a chromosome 7 pair. Herein, for example, recombinant L. sativa chromosome 7 is provided comprising an introgression fragment from a L. serriola donor.

The term “traditional breeding techniques” encompasses herein crossing, backcrossing, selfing, selection, double haploid production, embryo rescue, protoplast fusion, marker assisted selection, mutation breeding etc., all as known to the breeder (i.e, methods other than genetic modification/transformation/transgenic methods), by which, for example, a recombinant chromosome 1, 2 or 3 can be obtained, identified and/or transferred.

“Backcrossing” refers to a breeding method by which a (single) trait, such as a Fol4 and/or Fol-1 resistance QTL7.1, can be transferred from a (generally inferior) genetic background (e.g. a L. serriola; also referred to as “donor”) into a (generally superior) genetic background (also referred to as “recurrent parent”), e.g. L. sativa. An offspring of a cross (e.g. an F1 plant obtained by crossing a donor with a cultivated lettuce; or an F2 plant or F3 plant, etc., obtained from selfing the F1) is “backcrossed” to the parent with the superior genetic background, e.g. to the cultivated lettuce. After repeated backcrossing, the trait of the (generally inferior) genetic background will have been incorporated into the (generally superior) genetic background.

“Marker assisted selection” or “MAS” is a process of using the presence of molecular markers (such as SNP markers or INDEL markers), which are genetically linked to a particular locus or to a particular chromosome region (e.g. introgression fragment), to select plants for the presence of the specific locus or region (introgression fragment). For example, a molecular marker physically linked to a QTL7.1 (or a variant thereof), can be used to detect and/or select L. sativa plants comprising the Fol-4 and/or Fol-1 resistance QTL7.1 (or variant) on chromosome 7, or to detect and/or select other L. serriola donors comprising QTL7.1 or variants thereof. The closer the linkage of the molecular marker to the locus, the less likely it is that the marker is dissociated from the locus through meiotic recombination. Likewise, the closer two markers are linked to each other the less likely it is that the two markers will be separated from one another (and the more likely they will co-segregate as a unit).

“LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favor the presence of linkage and a LOD score greater than 3.0 is considered evidence for linkage. A LOD score of +3 indicates 1000 to 1 odds that the linkage being observed did not occur by chance. In the Examples provided, QTL7.1 was mapped with a LOD of 8.75 for the peak marker.

“Vegetative propagation”, “vegetative reproduction” or “clonal propagation” are used interchangeably herein and mean the method of taking part of a plant and allowing that plant part to form at least roots where plant part is, e.g., defined as or derived from (e.g. by cutting of) leaf, pollen, embryo, cotyledon, hypocotyl, cells, protoplasts, meristematic cell, root, root tip, pistil, anther, flower, shoot tip, shoot, stem, fruit, petiole, etc. When a whole plant is regenerated by vegetative propagation, it is also referred to as a vegetative propagation.

“Cell culture” or “tissue culture” refers to the in vitro culture of cells or tissues of a plant.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule.

A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

“Fine-mapping” refers to methods by which the position of a QTL can be determined more accurately (narrowed down) and by which the size of the introgression fragment comprising the QTL is reduced. For example Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

“Genotyping” methods or assays are methods whereby the genotype or allelic composition of a plant or plant part or seed can be determined. Bi-allelic genotyping assays, such as KASP-assays, can distinguish between two alleles or genotypes at a locus. Herein SNP marker genotyping for one or more of SNP_01 to SNP_10, especially for one or more of SNP_02 to SNP_09, is encompassed, which can e.g. be used to detect and/or select plants, seeds, plant parts or cells or tissues comprising QTL7.1 or a variant of QTL7.1 in wild L. serriola or in cultivated lettuce. For example, oligonucleotide primers can be made for use in a SNP genotyping assay for e.g. MAS (marker assisted selection) of QTL7.1 or a variant thereof.

“Oligonucleotides” or “oligos” or “oligonucleotide primers or probes” are short, single-stranded polymers of nucleic acid, e.g. at least 10, 11, 12, 13, 14, 15, 16, 17, 18, 19, 20, 21, 22, 23, 24 or more nucleotides in length. Oligos may be unmodified or modified with a variety of chemistries depending on their intended use, for example, the addition of 5′ or 3′ phosphate groups to enable ligation or block extension, respectively, labelling with radionucleotides or fluorophores and/or quenchers for use as probes, the incorporation of thiol, amino, or other reactive moieties to enable the covalent coupling of functional molecules such as enzymes, and extension with other linkers and spacers of diverse functionality. DNA oligos are the most commonly used, but RNA oligos are also available. The length of an oligo is usually designated by adding the suffix-mer. For example, an oligonucleotide with 19 nucleotides (bases) is called a 19-mer. For most uses, oligonucleotides are designed to base-pair with a strand of DNA or RNA. The most common use for oligonucleotides is as primers for PCR (polymerase chain reaction). Primers are designed with at least part of their sequence complementary to the sequence targeted for amplification. Optimal primer length for a complementary sequence is e.g. 18 to 22 nucleotides. Optimal primer sequences for PCR are usually determined by primer design software.

“DNA microarrays” are arrays which have many microscopic spots of DNA, usually oligonucleotides, bound on a solid support. Assay targets can be DNA, cDNA, or cRNA. Depending on the system, the hybridization of targets to specific spots is detected by fluorescence, chemiluminescence, or colloidal silver or gold. Microarrays are used for multiple applications such as simultaneous measurement of the expression of large numbers of genes, enabling genome-wide gene expression analysis, as well as genotyping studies using e.g. single-nucleotide polymorphism (SNP) or InDel analysis.

“Complementary strands” refer to two strands of complementary sequence, and may be referred to as sense (or plus) and anti-sense (or minus) strands for double stranded DNA. The sense/plus strand is, generally, the transcribed sequence of DNA (or the mRNA that was generated in transcription), while the anti-sense/minus strand is the strand that is complementary to the sense sequence. For any of the sequences provided herein only one strand of the sequence is given, but the complementary strand of the given strand is also encompassed herein. The complementary nucleotides of DNA are A complementary to T, and G complementary to C. The complementary nucleotides of RNA are A complementary to U, and G complementary to C.

### DETAILED DESCRIPTION

In the research that led to the present invention, a large set of wild lettuce accessions were screened for resistance against different races of Fusarium wilt, including races Fol-4 and Fol-1. One wild L. serriola accession was found, which had resistance against both of these races. Surprisingly, when mapping the resistance, only a single QTL was found, which was located on chromosome 7 of the lettuce reference genome. The QTL was therefore named QTL7.1.

The QTL7.1 of the L. serriola donor was crossed into a Fol-1 and Fol-4 susceptible L. sativa variety (recurrent parent), to generate an L. sativa line comprising QTL7.1 from the L. serriola donor. In the initial disease test, the L. sativa line had high resistance against Fol-4, but lower resistance than the L. serriola donor line against Fol-1. Without limiting the invention, it might be that the genetic background of the recurrent parent influences the expression of the Fol-1 resistance.

In one aspect of the invention a lettuce plant (Lactuca sativa L.) is provided comprising QTL7.1 (or a variant thereof) from L. serriola, whereby the QTL7.1 (or a variant thereof) confers high resistance against the Fusarium race Fol-4 and further intermediate resistance and/or high resistance against the race Fol-1.

Thus, in one aspect of the invention a lettuce plant (Lactuca sativa L.) is provided comprising QTL7.1 (or a variant of QTL7.1) from L. serriola, whereby the QTL7.1 (or variant thereof) confers resistance against the Fusarium race Fol-4 and resistance against the race Fol-1. Such dual resistance conferred by a single QTL is of particular advantage in breeding, as only a single QTL needs to be present, rather than two separate QTLs. This makes breeding simpler, e.g. by marker assisted breeding, and also allows stacking QTL7.1 (or variant QTL7.1) with other genes at different loci, e.g. other Fol-1 resistance genes.

The L. sativa line comprising QTL7.1 from the L. serriola donor was deposited by Nunhems B.V. (Napoleonsweg 152, 6083 AB Nunhem, The Netherlands) under the Budapest Treaty and received accession number NCIMB44108. Thus, in one aspect QTL7.1 is the QTL7.1 as present in, or obtainable from, or obtained from seeds deposited under NCIMB44108. The QTL7.1 (and the introgression fragment comprising the QTL7.1) is present in homozygous form in the deposited seeds.

The QTL has an “additive” effect, meaning that the effect on Fusarium wilt resistance of the QTL in homozygous form is the combined effect of the QTL in heterozygous form. Therefore, in one aspect the QTL7.1 is preferably present in homozygous form in the L. sativa plant, to confer the highest level of resistance. However, the QTL may also be present in heterozygous form in one aspect.

The QTL according to the invention, or an introgression fragment comprising the QTL, is obtainable from the genome of wild accessions of Lactuca serriola containing the QTL7.1 (or a variant thereof) which confers Fusarium wilt resistance, including but not limited to, wild L. serriola plants obtainable from e.g. the CGN collection or the US ARS-GRIN collection. The QTL7.1 of the invention is obtainable from e.g. the resistant L. serriola accession which has been used as donor to introgress QTL7.1 into L. sativa, representative seeds of which have been deposited under NCIMB44108.

The present invention, thus, relates to a cultivated lettuce plant comprising a QTL, or an introgression fragment from L. serriola comprising a QTL, which confers a Fol-1 and/or Fol-4 resistance, wherein the QTL is located on chromosome 7 of the L. sativa genome, in the region between nucleotide 64.382.881 and nucleotide 69.375.685 of chromosome 7 of the reference genome of lettuce, which is genome V11, found in the world wide web at ncbi.nlm.nih.gov/data-hub/genome/?taxon=4236.

The resistance conferring QTL7.1, or a variant thereof from e.g. a different L. serriola accession, is located on an L. serriola introgression fragment, located between the flanking markers SNP_01 and SNP_10. The physical location of the QTL on chromosome 7 is, thus, in-between the nucleotide (or base) for SNP_01 and the nucleotide for SNP_10 on the chromosome 7 of L. sativa.

As SNP_01 corresponds to nucleotide 64.382.881 of chromosome 7 of the L. sativa V11 reference genome and SNP_10 corresponds to nucleotide 69.375.685 of chromosome 7 of the L. sativa V11 reference genome, the QTL7.1 (or variant thereof, or the introgression fragment comprising QTL7.1 or variant) is located in-between nucleotide 64.382.881 and nucleotide 69.375.685 of chromosome 7 of the L. sativa V11 reference genome. Which nucleotide (A, T, C or G) is actually present at the SNP_01 position and at SNP_10 position on chromosome 7 is not relevant.

In total 10 SNP markers (Single Nucleotide Polymorphism markers) are provided herein which are linked to QTL7.1 (or a variant thereof), or which are ‘indicative’ of QTL7.1 or variant QTL7.1 (or which are ‘indicative of the introgression fragment’ comprising QTL7.1 or variant) and one or more of these SNP markers can be used to identify plants, seeds and plant parts comprising QTL7.1 (or a variant). These 10 SNP markers are referred to as SNP_01 tot SNP_10, with SNP_07 being the peak marker in the QTL mapping and SNP_01 and SNP_10 being ‘flanking’ markers either side of the region comprising QTL7.1 (or a variant) from the L. serriola donor. See also FIG. 4. The flanking markers, SNP_01 and SNP_10, thus delimit the region on chromosome 7 between which QTL7.1 (or a variant QTL7.1) is found.

The SNP markers are the ‘single nucleotide polymorphisms’ which have either the L. serriola donor nucleotide or a different nucleotide, e.g. a nucleotide of the recurrent parent. For all 10 SNP markers the right and left sequence flanking the SNP marker (i.e. the polymorphic nucleotide) is provided herein from the wild L. serriola donor (SEQ ID NO: 11 to SEQ ID NO: 20), from the L. sativa reference genome V11 (SEQ ID NO: 21 to SEQ ID NO: 30) and as a consensus sequence of both of these sequences, which takes the nucleotide differences in the genomic DNA sequence flanking the SNP nucleotide between the wild L. serriola donor and the L. sativa reference genome into account (SEQ ID NO: 1 to SEQ ID NO: 10). For example nucleotide 67 of SEQ ID NO: 11 is a ‘C’, while it is an ‘A’ in SEQ ID NO: 21, whereby the consensus sequence of SEQ ID NO: 1 has an ‘M’ (indicating C or A) at nucleotide 67.

In one aspect a cultivated lettuce (L. sativa) plants, seeds, plant parts, cells and/or tissues is provided which comprises an introgression fragment from a wild L. serriola donor, whereby the introgression fragment comprises QTL7.1 (or a variant thereof), which is located physically in the region starting at nucleotide 64.382.881 (corresponding to SNP_01 at nucleotide 101 of SEQ ID NO: 1, 11 or 21) and ending at nucleotide 69.375.685 (corresponding to SNP_10 at nucleotide 101 of SEQ ID NO: 10, 20 or 30) of chromosome 7, with reference to the chromosome 7 of the V11 reference genome.

In one aspect the introgression fragment comprising the QTL7.1 (or a variant QTL7.1) comprises the L. serriola donor nucleotide for at least 1, 2, 3, 4, 5, 6, 7, 8, 9 or 10 SNP markers selected from SNP_01 to SNP_10, especially for at least 1, 2, 3, 4, 5, 6, 7 or 8 SNP markers selected from SNP_02 to SNP_09, i.e. selected from: SNP_02 at nucleotide 101 of SEQ ID NO: 2 or at the equivalent position in a sequence comprising at least 95% sequence identity to SEQ ID NO:2, SNP_03 at nucleotide 101 of SEQ ID NO: 3 or at the equivalent position in a sequence comprising at least 95% sequence identity to SEQ ID NO: 3, SNP_04 at nucleotide 101 of SEQ ID NO: 4 or at the equivalent position in a sequence comprising at least 95% sequence identity to SEQ ID NO: 4, SNP_05 at nucleotide 101 of SEQ ID NO: 5 or at the equivalent position in a sequence comprising at least 95% sequence identity to SEQ ID NO: 5, SNP_06 at nucleotide 101 of SEQ ID NO: 6 or at the equivalent position in a sequence comprising at least 95% sequence identity to SEQ ID NO: 6, SNP_07 at nucleotide 101 of SEQ ID NO: 7 or at the equivalent position in a sequence comprising at least 95% sequence identity to SEQ ID NO: 7, SNP_08 at nucleotide 101 of SEQ ID NO: 8 or at the equivalent position in a sequence comprising at least 95% sequence identity to SEQ ID NO: 8, SNP_09 at nucleotide 101 of SEQ ID NO: 9 or at the equivalent position in a sequence comprising at least 95% sequence identity to SEQ ID NO: 9.

In one aspect said at least 1, 2, 3, 4, 5, 6, 7 or 8 markers include the L. serriola donor nucleotide for at least the following SNP marker: SNP_07 at nucleotide 101 of SEQ ID NO: 7 or at the equivalent position in a sequence comprising at least 95% sequence identity to SEQ ID NO: 7.

In another aspect said at least 1, 2, 3, 4, 5, 6, 7 or 8 markers include the L. serriola donor nucleotide for at least one or more of the following SNP markers: SNP_07 at nucleotide 101 of SEQ ID NO: 7 or at the equivalent position in a sequence comprising at least 95% sequence identity to SEQ ID NO: 7, SNP_06 at nucleotide 101 of SEQ ID NO: 6 or at the equivalent position in a sequence comprising at least 95% sequence identity to SEQ ID NO: 6 and/or SNP_08 at nucleotide 101 of SEQ ID NO: 8 or at the equivalent position in a sequence comprising at least 95% sequence identity to SEQ ID NO: 8.

In another aspect said at least 1, 2, 3, 4, 5, 6, 7 or 8 markers include the L. serriola donor nucleotide for at least one or more of the following SNP markers: SNP_07 at nucleotide 101 of SEQ ID NO: 7 or at the equivalent position in a sequence comprising at least 95% sequence identity to SEQ ID NO: 7, SNP_05 at nucleotide 101 of SEQ ID NO: 5 or at the equivalent position in a sequence comprising at least 95% sequence identity to SEQ ID NO: 5, SNP_06 at nucleotide 101 of SEQ ID NO: 6 or at the equivalent position in a sequence comprising at least 95% sequence identity to SEQ ID NO: 6, SNP_08 at nucleotide 101 of SEQ ID NO: 8 or at the equivalent position in a sequence comprising at least 95% sequence identity to SEQ ID NO: 8 and/or SNP_09 at nucleotide 101 of SEQ ID NO: 9 or at the equivalent position in a sequence comprising at least 95% sequence identity to SEQ ID NO: 9.

In one aspect a cultivated Lactuca sativa plant comprising an introgression fragments from a wild L. serriola donor comprising QTL7.1 (or a variant thereof) in homozygous or heterozygous form is provided, wherein said introgression fragment comprises
a Quantitative Trait Locus (QTL7.1, or a variant) located between the Single Nucleotide Polymorphism marker SNP_01 at nucleotide 101 of SEQ ID NO: 1 (or of a variant of SEQ ID NO: 1) and the Single Nucleotide Polymorphism marker SNP_10 at nucleotide 101 of SEQ ID NO: 10 (or of a variant of SEQ ID NO: 10), which QTL confers resistance against Fusarium oxysporum f.sp. lactucae (Fol) race 1 (Fol-1) and/or race 4 (Fol-4) when in homozygous or heterozygous form.

In one aspect QTL7.1 (or a variant thereof, or the introgression fragment comprising the QTL) is present in heterozygous form in a cultivated Lactuca sativa plant, cell or tissue.

In another aspect QTL7.1 (or a variant thereof, or the introgression fragment comprising the QTL) is present in homozygous form in a Lactuca sativa plant, seed, plant part, cell or tissue.

In one aspect the L. serriola donor of the QTL is selected from a L. serriola donor comprising the same SNP haplotype for at least 2, 3, 4, 5, 6, 7, 8, 9 or 10 SNP markers for the QTL as present in seeds deposited under NCIMB44108, deposited under the Budapest Treaty on Feb. 1, 2023 by Nunhems B.V. Therefore, in one aspect seeds, or progeny of seeds, deposited under NCIMB44108 (comprising QTL7.1) can be used as donor for QTL7.1, or any other L. serriola donor which is resistant against Fol-1 and/or Fol-4 can be used, which wild L. serriola donor has the same SNP haplotype for at least 2, 3, 4, 5, 6, 7, 8, 9 or 10 SNP markers described herein for the QTLs present in NCIMB44108 can be used as donors for QTL7.1 or a variant QTL7.1.

Thus, the present invention relates to a cultivated Lactuca sativa plant comprising a Fol-4 and/or Fol-1 resistance-conferring QTL introgressed from a Fol-4 and/or Fol-1 resistant L. serriola accession, especially a dual resistant L. serriola accession. Thus, the increased resistance is conferred by an introgression fragment on cultivated lettuce chromosome 7 (comprising QTL7.1 or a variant thereof), wherein said introgression fragment is from an L. serriola accession, referred to as the ‘donor’ of the QTL.

When reference is made herein to an introgression fragment on chromosome 7 having a Fol-4 and/or Fol-1 resistance-conferring QTL, this encompasses various sizes of introgression fragments, e.g. the fragment as found in NCIMB 44108 comprising the donor SNP nucleotide or all SNP markers linked to the QTL (SNP_01 to SNP_10, or SNP_02 to SNP_09, or any marker in between these), but also smaller introgression fragments (comprising less than these 10 or 8 SNP markers such as only e.g. 2, 3, 4, 5, 6, 7, 8 or 9 of the SNP markers), where however the fragment remains large enough to confer Fol-4 and/or Fol-1 resistance (compared to the control or genetic control) when the introgression fragment is in heterozygous or preferably in homozygous form in the cultivated lettuce genome. In other words, the fragment retains QTL7.1, or a variant thereof, i.e. it still confers significantly enhanced Fol-4 and/or Fol-1 resistance (compared to the control, e.g. the genetic control) when the introgression fragment is in heterozygous or preferably in homozygous form in the cultivated lettuce genome.

Further, when reference is made herein to an introgression fragment on chromosome 7 having a Fol-4 and/or Fol-1 resistance-conferring QTL this encompasses introgression fragments from various donors which comprise the same or variant QTL7.1 of the QTL7.1 present in e.g. NCIMB 44108. Such variant QTL7.1 has the same SNP haplotype (or SNP genotype) for at least 2, 3, 4, 5, 6, 7, 8, 9 or all 10 of the SNP makers, described e.g. in Table 1, wherein said at least 2, 3, 4, 5, 6, 7 or 8 markers are preferably selected from SNP_02 to SNP_09. For example, L. serriola donors available at the ARS-GRIN collection in the US (see world wide web at npgsweb.ars-grin.gov/gringlobal/search) or available at the Center for Genetic Resources, Wageningen University,//cgngenis.wur.nl/(CGN), or other accessions, which comprise the same SNP haplotype or genotype for at least 2, 3, 4, 5, 6, 7, 8, 9 or all 10 markers. Preferably the donor which comprises the same SNP haplotype or genotype for at least 2, 3, 4, 5, 6, 7, 8, 9 or all 10 markers also has Fol-4 and/or Fol-1 resistance, e.g. HR or IR resistance against Fol-4 and/or HR or IR resistance against Fol-1. This can be tested as described in the Examples. Different donors may, for example, first be selected based on their resistance phenotype and then screened for the presence of QTL7.1 (or a variant thereof) using one or more or all of SNP_01 to SNP_10 or of SNP_02 to SNP_09, or the other way round, they may first be screened for the presence of QTL7.1 (or a variant thereof) and then tested for Fol-4 and/or Fol-1 resistance phenotype.

“SNP_01” is to be understood in context with the present invention to be a SNP at position 101 in SEQ ID NO: 1, or at the equivalent position in a sequence comprising at least 95%, 96%, 97%, 98% or 99% sequence identity to SEQ ID NO: 1, such as SEQ ID NO: 11 (which comprises SNP_01 at nucleotide 101 of SEQ ID NO: 11) or SEQ ID NO: 21 (which comprises SNP_01 at nucleotide 101 of SEQ ID NO: 21). The position of SNP_01 on chromosome 7 (of the V11 reference genome of lettuce) is found in Table 1, 2 and 3. As SNP_01 is a SNP marker flanking the genomic region comprising QTL7.1 (or a variant thereof), the SNP_01 nucleotide may in one aspect be either from the L. serriola donor or from the L. sativa plant. The SNP_01 nucleotide may, thus, be either an Adenine (indicative of the resistant L. serriola donor) or a Guanine, Thymine or Cytosine (indicative of the L. sativa genome).

“SNP_02” is to be understood in context with the present invention to be a SNP at position 101 in SEQ ID NO: 2, or at the equivalent position in a sequence comprising at least 95%, 96%, 97%, 98% or 99% sequence identity to SEQ ID NO: 2, such as SEQ ID NO: 12 (which comprises SNP_02 at nucleotide 101 of SEQ ID NO: 12) or SEQ ID NO: 22 (which comprises SNP_02 at nucleotide 101 of SEQ ID NO: 22). The position of SNP_02 on chromosome 7 (of the V11 reference genome of lettuce) is found in Table 1, 2 and 3. The SNP_02 nucleotide is in one aspect from the L. serriola donor, i.e. it is a Guanine (indicative of the resistant L. serriola donor).

“SNP_03” is to be understood in context with the present invention to be a SNP at position 101 in SEQ ID NO: 3, or at the equivalent position in a sequence comprising at least 95%, 96%, 97%, 98% or 99% sequence identity to SEQ ID NO: 3, such as SEQ ID NO: 13 (which comprises SNP_03 at nucleotide 101 of SEQ ID NO: 13) or SEQ ID NO: 23 (which comprises SNP_03 at nucleotide 101 of SEQ ID NO: 23). The position of SNP_03 on chromosome 7 (of the V11 reference genome of lettuce) is found in Table 1, 2 and 3. The SNP_03 nucleotide is in one aspect from the L. serriola donor, i.e. it is a Cytosine (indicative of the resistant L. serriola donor).

“SNP_04” is to be understood in context with the present invention to be a SNP at position 101 in SEQ ID NO: 4, or at the equivalent position in a sequence comprising at least 95%, 96%, 97%, 98% or 99% sequence identity to SEQ ID NO: 4, such as SEQ ID NO: 14 (which comprises SNP_04 at nucleotide 101 of SEQ ID NO: 14) or SEQ ID NO: 24 (which comprises SNP_04 at nucleotide 101 of SEQ ID NO: 24). The position of SNP_04 on chromosome 7 (of the V11 reference genome of lettuce) is found in Table 1, 2 and 3. The SNP_04 nucleotide is in one aspect from the L. serriola donor, i.e. it is an Adenine (indicative of the resistant L. serriola donor).

“SNP_05” is to be understood in context with the present invention to be a SNP at position 101 in SEQ ID NO: 5, or at the equivalent position in a sequence comprising at least 95%, 96%, 97%, 98% or 99% sequence identity to SEQ ID NO: 5, such as SEQ ID NO: 15 (which comprises SNP_05 at nucleotide 101 of SEQ ID NO: 15) or SEQ ID NO: 25 (which comprises SNP_05 at nucleotide 101 of SEQ ID NO: 25). The position of SNP_05 on chromosome 7 (of the V11 reference genome of lettuce) is found in Table 1, 2 and 3. The SNP_05 nucleotide is in one aspect from the L. serriola donor, i.e. it is a Guanine (indicative of the resistant L. serriola donor).

“SNP_06” is to be understood in context with the present invention to be a SNP at position 101 in SEQ ID NO: 6, or at the equivalent position in a sequence comprising at least 95%, 96%, 97%, 98% or 99% sequence identity to SEQ ID NO: 6, such as SEQ ID NO: 16 (which comprises SNP_06 at nucleotide 101 of SEQ ID NO: 16) or SEQ ID NO: 26 (which comprises SNP_06 at nucleotide 101 of SEQ ID NO: 26). The position of SNP_06 on chromosome 7 (of the V11 reference genome of lettuce) is found in Table 1, 2 and 3. The SNP_06 nucleotide is in one aspect from the L. serriola donor, i.e. it is a Guanine (indicative of the resistant L. serriola donor).

“SNP_07” is to be understood in context with the present invention to be a SNP at position 101 in SEQ ID NO: 7, or at the equivalent position in a sequence comprising at least 95%, 96%, 97%, 98% or 99% sequence identity to SEQ ID NO: 7, such as SEQ ID NO: 17 (which comprises SNP_07 at nucleotide 101 of SEQ ID NO: 17) or SEQ ID NO: 27 (which comprises SNP_07 at nucleotide 101 of SEQ ID NO: 27). The position of SNP_07 on chromosome 7 (of the V11 reference genome of lettuce) is found in Table 1, 2 and 3. The SNP_07 nucleotide is in one aspect from the L. serriola donor, i.e. it is a Thymine (indicative of the resistant L. serriola donor).

“SNP_08” is to be understood in context with the present invention to be a SNP at position 101 in SEQ ID NO: 8, or at the equivalent position in a sequence comprising at least 95%, 96%, 97%, 98% or 99% sequence identity to SEQ ID NO: 8, such as SEQ ID NO: 18 (which comprises SNP_08 at nucleotide 101 of SEQ ID NO: 18) or SEQ ID NO: 28 (which comprises SNP_08 at nucleotide 101 of SEQ ID NO: 28). The position of SNP_08 on chromosome 7 (of the V11 reference genome of lettuce) is found in Table 1, 2 and 3. The SNP_08 nucleotide is in one aspect from the L. serriola donor, i.e. it is a Thymine (indicative of the resistant L. serriola donor).

“SNP_09” is to be understood in context with the present invention to be a SNP at position 101 in SEQ ID NO: 9, or at the equivalent position in a sequence comprising at least 95%, 96%, 97%, 98% or 99% sequence identity to SEQ ID NO: 9, such as SEQ ID NO: 19 (which comprises SNP_09 at nucleotide 101 of SEQ ID NO: 19) or SEQ ID NO: 29 (which comprises SNP_09 at nucleotide 101 of SEQ ID NO: 29). The position of SNP_09 on chromosome 7 (of the V11 reference genome of lettuce) is found in Table 1, 2 and 3. The SNP_09 nucleotide is in one aspect from the L. serriola donor, i.e. it is an Adenine (indicative of the resistant L. serriola donor).

“SNP_10” is to be understood in context with the present invention to be a SNP at position 101 in SEQ ID NO: 10, or at the equivalent position in a sequence comprising at least 95%, 96%, 97%, 98% or 99% sequence identity to SEQ ID NO: 10, such as SEQ ID NO: 20 (which comprises SNP_10 at nucleotide 101 of SEQ ID NO: 20) or SEQ ID NO: 30 (which comprises SNP_10 at nucleotide 101 of SEQ ID NO: 30). The position of SNP_10 on chromosome 7 (of the V11 reference genome of lettuce) is found in Table 1, 2 and 3. As SNP_10 is a SNP marker flanking the genomic region comprising QTL7.1 (or a variant thereof), the SNP_10 nucleotide may in one aspect be either from the L. serriola donor or from the L. sativa plant. The SNP_10 nucleotide may, thus, be either an Guanine (indicative of the resistant L. serriola donor) or a Adenine, Thymine or Cytosine (indicative of the L. sativa genome).

Thus, when referring herein to a SNP nucleotide at nucleotide 101 of a particular SEQ ID NO, this encompasses the SNP nucleotide at the equivalent position in a sequence comprising at least 95%, 96%, 97%, 98%, 99% sequence identity to the SEQ ID NO (which sequence is referred to herein as a ‘variant sequence’ of the specific SEQ ID NO, or alternatively as a sequence comprising ‘substantial sequence identity’ to the SEQ ID NO). Pairwise sequence alignment can be used to e.g. determine whether the nucleotide is present at the equivalent position in such a sequence.

### QTL7.1 and Variants of QTL7.1 on Chromosome 7

Thus, in one aspect a cultivated L. sativa plant is provided comprising an introgression fragment from a wild L. serriola donor, wherein the introgression fragment comprises QTL7.1, or a variant thereof, and wherein the introgression fragment comprises all or part of the region starting at nucleotide (or base) 64382881 of chromosome 7 (corresponding to SNP_01) and ending at nucleotide (or base) 69375685 of chromosome 7 (corresponding to SNP_10). In other words, all or part of the region starting at nucleotide 64382881 of chromosome 7 (SNP_01) and ending at nucleotide 69375685 of chromosome 7 (SNP_10) is, in one aspect, from a wild L. serriola donor and comprises QTL7.1 or a variant thereof. In particular this region comprises the L. serriola donor nucleotide for one or more or all of SNP_02 to SNP_09. Which sub-region in-between SNP_01 and SNP_10 contains QTL7.1 (or a variant) can be identified by e.g. fine-mapping. The peak marker of QTL7.1 is SNP_07, so if QTL7.1 (or a variant) is found to be located in between SNP_05 and SNP_09, then the plant of the invention only needs to comprise the introgression region starting at nucleotide 66347850 of chromosome 7 (SNP_05) and ending at nucleotide 69331664 (SNP_09) of chromosome 7.

In one aspect QTL7.1 (or a variant thereof) is located in-between marker SNP_01 at nucleotide 101 of SEQ ID NO: 1 (or at nucleotide 101 in a variant sequence of SEQ ID NO: 1) and marker SNP_10 at nucleotide 101 of SEQ ID NO: 10 (or at nucleotide 101 in a variant sequence of SEQ ID NO: 10).

In another aspect QTL7.1 (or a variant thereof) is located in-between marker SNP_02 at nucleotide 101 of SEQ ID NO: 2 (or at nucleotide 101 in a variant sequence of SEQ ID NO: 2) and marker SNP_09 at nucleotide 101 of SEQ ID NO: 09 (or at nucleotide 101 in a variant sequence of SEQ ID NO: 9).

In a further aspect QTL7.1 (or a variant thereof) is located in-between marker SNP_03 at nucleotide 101 of SEQ ID NO: 3 (or at nucleotide 101 in a variant sequence of SEQ ID NO: 3) and marker SNP_09 at nucleotide 101 of SEQ ID NO: 09 (or at nucleotide 101 in a variant sequence of SEQ ID NO: 9). In a further aspect QTL7.1 (or a variant thereof) is located in-between marker SNP_04 at nucleotide 101 of SEQ ID NO: 4 (or at nucleotide 101 in a variant sequence of SEQ ID NO: 4) and marker SNP_09 at nucleotide 101 of SEQ ID NO: 09 (or at nucleotide 101 in a variant sequence of SEQ ID NO: 9). In yet another aspect QTL7.1 (or a variant thereof) is located in-between marker SNP_05 at nucleotide 101 of SEQ ID NO: 5 (or at nucleotide 101 in a variant sequence of SEQ ID NO: 5) and marker SNP_09 at nucleotide 101 of SEQ ID NO: 09 (or at nucleotide 101 in a variant sequence of SEQ ID NO: 9). In still a further aspect QTL7.1 (or a variant thereof) is located in-between marker SNP_06 at nucleotide 101 of SEQ ID NO: 6 (or at nucleotide 101 in a variant sequence of SEQ ID NO: 6) and marker SNP_08 at nucleotide 101 of SEQ ID NO: 08 (or at nucleotide 101 in a variant sequence of SEQ ID NO: 8).

In one aspect the introgression fragment of the invention (comprising QTL7.1 or a variant thereof) is a fragment comprising a smaller fragment (part) of the region starting at nucleotide (or base) 64442022 of chromosome 7 (SNP_02 location) and ending at nucleotide (or base) 69331664 of chromosome 7 (SNP_09 location), e.g. having a size of e.g. 4.5 Mb, 4.0 Mb, 3.5 Mb, 3.0 Mb, 2.5 Mb, 2.0 Mb, 1.5 Mb, 1.0 Mb, 0.5 Mb, 100 kb, 50 kb, 35 kb, 30 kb, 20 kb, or less and comprising the QTL or a variant thereof. In one aspect the part is at least 5 kb, 10 kb, 20 kb in size, or more.

In one aspect the introgression fragment on chromosome 7 comprising QTL7.1, or a variant thereof, is obtainable by crossing a plant grown from NCIMB44108 with another lettuce plant, especially a cultivated lettuce plant.

In one aspect the cultivated lettuce plant of the invention comprising QTL7.1, or a variant thereof, is a plant wherein said introgression fragment on chromosome 7 is obtainable by crossing a plant grown from seeds deposited under accession number NCIMB44108 with another lettuce plant. Thus, in one aspect the QTL is the QTL present in seeds deposited under accession number NCIMB44108.

In a further aspect the cultivated lettuce plant of the invention comprising QTL7.1, or a variant thereof, is a plant wherein said introgression fragment on chromosome 7 is obtainable by crossing a plant comprising the same donor SNP haplotype (or donor SNP genotype) for at least 2, 3, 4, 5, 6, 7, 8, 9 or 10 SNP markers linked to the QTL (i.e. SNP_01 to SNP_10, or SNP_02 to SNP_09, for QTL7.1 as shown in Table 1) with another lettuce plant, especially with an elite breeding line. Thus, in one aspect the QTL is the variant QTL present in wild L. serriola donor accessions which comprise the same donor SNP haplotype (or donor genotype) for at least 2, 3, 4, 5, 6, 7, 8, 9 or 10 SNP markers e.g. as present in NCIMB44108. Preferably the donor also comprises a resistance phenotype against Fol-4 and/or Fol-1 races, preferably dual resistance.

When referring to the SNP markers herein, which are indicative of the presence of the introgression fragment (and the Fol-4 and/or Fol-1 resistance QTL present on the introgression fragment), it is understood that the donor SNP genotype or haplotype which is indicative of the introgression fragment is referred to, i.e. the donor SNP genotype or haplotype as provided e.g. in Tables 1 to 3. It is noted that the SNP marker genotype can distinguish between the introgression fragment being in homozygous or heterozygous form. In homozygous form the SNP nucleotide is identical, while in heterozygous form the nucleotide is not identical. The SNP genotype of the ‘wild type’ chromosome lacking the introgression fragment is the other haplotype, e.g. the haplotype of the recurrent parent). So, e.g. the genotype of SNP_07 indicative of the introgression fragment comprising QTL7.1 is ‘TC’ (QTL7.1/wt) or ‘TT’ (QTL7.1/QTL7.1) while the SNP genotype indicative of the wild type/genetic control (lacking the introgression fragment) is e.g. ‘CC’ (wt/wt). This can also be written as genotype TX′ (QTL7.1/wt) or ‘TT’ (QTL7.1/QTL7.1) while the SNP genotype indicative of the wild type/genetic control (lacking the introgression fragment) is e.g. ‘XX’ (wt/wt). X may be any nucleotide (A, T, C or G), especially any other nucleotide than the donor nucleotide (e.g. A, C or G). Thus, when referring to a plant or plant part (e.g. cell) comprising the introgression fragment in homozygous or heterozygous form, it is understood that the SNP markers linked to the introgression fragment have the corresponding SNP genotype or haplotype.

So, in one aspect, a cultivated L. sativa plant is provided comprising an introgression fragment on chromosome 7 in homozygous or heterozygous form, wherein said introgression fragment confers Fol-4 and/or Fol-1 resistance compared to the lettuce plant lacking the introgression fragment on chromosome 7, e.g. the genetic control or control variety, when grown under the same conditions.

The plants of the invention, therefore, comprise a genome of cultivated lettuce, with at least one (heterozygous) or two (homozygous) recombinant chromosomes 7. The recombinant chromosomes comprise a fragment of a L. serriola donor, which is easily distinguishable from the cultivated L. sativa genome by molecular marker analysis (e.g. of one or more of SNP_01 to SNP_10, especially of SNP_02 to SNP_09), whole genome sequencing, chromosome painting and similar techniques.

In one aspect the introgression fragment on chromosome 7 is from a L. serriola donor, and comprises the QTL7.1, or a variant thereof, and comprises all or part of the region starting at nucleotide SNP_01 and ending at SNP_10, especially all or part of the region starting at SNP_02 and ending at SNP_09.

Thus, the introgression fragment comprises the QTL7.1, or a variant thereof, and one or more or all (e.g. 2, 3, 4, 5, 6, 7, 8, 9 or 10) SNP markers of the donor selected from SNP_01 to SNP_10 or selected from SNP_02 to SNP_09 as shown in e.g. Table 1.

In one aspect the introgression fragment comprises QTL7.1 (or a variant) and one or more or all of SEQ ID NO: 1 to SEQ ID NO: 10, or of SEQ ID NO: 2 to SEQ ID NO: 9, or variant sequences thereof comprising at least 95% sequence identity to any one of these sequences, e.g. one or more or all of SEQ ID NO: 11 to SEQ ID NO: 20, or of SEQ ID NO: 12 to SEQ ID NO: 19; or one or more or all of SEQ ID NO: 21 to SEQ ID NO: 30, or of SEQ ID NO: 22 to SEQ ID NO: 29.

In one aspect the presence of the introgression fragment on chromosomes 7 comprising QTL7.1 (or a variant) in the genome of the plant, seed, plant part or plant cell or plant tissue (or in the DNA extracted therefrom) is detectable by a molecular marker assay (e.g. a genotyping assay) which detects one or more molecular markers of the introgression fragment, especially the donor SNP haplotype (or genotype) for at least 2, 3, 4, 5, 6, 7, 8, 9, 10 or more of SNP_01 to SNP_10, at nucleotide 101 of SEQ ID NO: 1 to 10 (or at nucleotide 101 of variant sequences of these), respectively, especially the donor SNP haplotype (or genotype) for at least 2, 3, 4, 5, 6, 7 or 8 markers of SNP_02 to SNP_09. However, as mentioned, other techniques may be used, e.g. the SNP genotype of the markers may also be determined by sequencing or by using alternative markers located in between the SNP markers provided herein.

In one aspect the presence of the introgression fragment on chromosomes 7 comprising QTL7.1 (or a variant) in the genome of the plant, seed, plant part or plant cell or plant tissue (or in the DNA extracted therefrom) is detectable by detecting the presence of one or more or all of SEQ ID NO: 1 to 10 or of SEQ ID NO: 2 to 9, one or more or all of SEQ ID NO: 11 to 20 or of SEQ ID NO: 12 to 19, or one or more or all of SEQ ID NO: 21 to 30 or of SEQ ID NO: 22 to 29.

When reference is made herein to one or more molecular markers or sequences being “detectable” by e.g. a molecular marker assay, this means of course that the plant or plant part comprises the one or more markers or sequences in its genome, as the marker or sequence would otherwise not be detectable.

### Lettuce Plants Comprising an Introgression Fragment on Chromosome 7 (OTL 7.1)

In one aspect a cultivated L. sativa plant comprising an introgression fragment from a L. serriola donor on chromosome 7 in homozygous or heterozygous form is provided, wherein said introgression fragment comprises a Quantitative Trait Locus (QTL) located between the Single Nucleotide Polymorphism marker SNP_01 at nucleotide 101 of SEQ ID NO: 1 (or at nucleotide 101 of a variant of SEQ ID NO: 1 comprising at least 95% sequence identity to SEQ ID NO: 1) and the Single Nucleotide Polymorphism marker SNP_10 at nucleotide 101 of SEQ ID NO: 10 (or at nucleotide 101 of a variant of SEQ ID NO: 10 comprising at least 95% sequence identity to SEQ ID NO:), which QTL confers Fol-4 and/or Fol-1 resistance. In one aspect the QTL is located between base 64382881 (SNP_01) and base 69375685 (SNP_10) of chromosome 7.

Thus, in one aspect QTL7.1 (or a variant thereof) is located in the region between SNP_01 in SEQ ID NO: 1 (or in a variant thereof) and SNP_10 in SEQ ID NO: 10 (or in a variant thereof).

Provided is, in one aspect, a Lactuca sativa plant comprising an introgression fragment from Lactuca serriola on chromosome 7, which comprises a Quantitative Trait Locus (QTL) that confers resistance against Fusarium oxysporum f.sp. lactucae races Fol-4 and/or Fol-1 and wherein said introgression fragment on chromosome 7 comprises QTL7.1 in the region starting at nucleotide 64442022 and ending at nucleotide 69331664 of chromosome 7, wherein said introgression fragment comprises at least 1, 2, 3, 4, 5, 6, 7 or 8 of the following markers:
a) the GX or GG genotype for the Single Nucleotide Polymorphism marker SNP_02 at nucleotide 101 of SEQ ID NO: 2 (or at nucleotide 101 in a variant thereof comprising at least 95% sequence identity to SEQ ID NO: 2);
b) the CX or CC genotype for the Single Nucleotide Polymorphism marker SNP_03 at nucleotide 101 of SEQ ID NO: 3 (or at nucleotide 101 in a variant thereof comprising at least 95% sequence identity to SEQ ID NO: 3);
c) the AX or AA genotype for the Single Nucleotide Polymorphism marker SNP_04 at nucleotide 101 of SEQ ID NO: 4 (or at nucleotide 101 in a variant thereof comprising at least 95% sequence identity to SEQ ID NO: 4);
d) the GX or GG genotype for the Single Nucleotide Polymorphism marker SNP_05 at nucleotide 101 of SEQ ID NO: 5 (or at nucleotide 101 in a variant thereof comprising at least 95% sequence identity to SEQ ID NO: 5);
e) the GX or GG genotype for the Single Nucleotide Polymorphism marker SNP_06 at nucleotide 101 of SEQ ID NO: 6 (or at nucleotide 101 in a variant thereof comprising at least 95% sequence identity to SEQ ID NO: 6);
f) the TX or TT genotype for the Single Nucleotide Polymorphism marker SNP_07 at nucleotide 101 of SEQ ID NO: 7 (or at nucleotide 101 in a variant thereof comprising at least 95% sequence identity to SEQ ID NO: 7);
g) the TX or TT genotype for the Single Nucleotide Polymorphism marker SNP_08 at nucleotide 101 of SEQ ID NO: 8 (or at nucleotide 101 in a variant thereof comprising at least 95% sequence identity to SEQ ID NO: 8);
h) the AX or AA genotype for the Single Nucleotide Polymorphism marker SNP_09 at nucleotide 101 of SEQ ID NO: 9 (or at nucleotide 101 in a variant thereof comprising at least 95% sequence identity to SEQ ID NO: 9);

Therefore, in one aspect a L. sativa plant is provided comprising an introgression fragment on chromosome 7 in homozygous or heterozygous form, wherein said introgression fragment confers Fol-4 and/or Fol-1 resistance (compared to the plant lacking the introgression fragment, e.g. the recurrent parent or control) and wherein said introgression fragment comprises the SNP marker haplotype or genotype of at least 1, 2, 3, 4, 5, 6, 7 or 8 of the SNP markers selected from the group consisting of:
a) the GX or GG genotype for the Single Nucleotide Polymorphism marker SNP_02 at nucleotide 101 of SEQ ID NO: 2 (or at nucleotide 101 in a variant thereof comprising at least 95% sequence identity to SEQ ID NO: 2);
b) the CX or CC genotype for the Single Nucleotide Polymorphism marker SNP_03 at nucleotide 101 of SEQ ID NO: 3 (or at nucleotide 101 in a variant thereof comprising at least 95% sequence identity to SEQ ID NO: 3);
c) the AX or AA genotype for the Single Nucleotide Polymorphism marker SNP_04 at nucleotide 101 of SEQ ID NO: 4 (or at nucleotide 101 in a variant thereof comprising at least 95% sequence identity to SEQ ID NO: 4);
d) the GX or GG genotype for the Single Nucleotide Polymorphism marker SNP_05 at nucleotide 101 of SEQ ID NO: 5 (or at nucleotide 101 in a variant thereof comprising at least 95% sequence identity to SEQ ID NO: 5);
e) the GX or GG genotype for the Single Nucleotide Polymorphism marker SNP_06 at nucleotide 101 of SEQ ID NO: 6 (or at nucleotide 101 in a variant thereof comprising at least 95% sequence identity to SEQ ID NO: 6);
f) the TX or TT genotype for the Single Nucleotide Polymorphism marker SNP_07 at nucleotide 101 of SEQ ID NO: 7 (or at nucleotide 101 in a variant thereof comprising at least 95% sequence identity to SEQ ID NO: 7);
g) the TX or TT genotype for the Single Nucleotide Polymorphism marker SNP_08 at nucleotide 101 of SEQ ID NO: 8 (or at nucleotide 101 in a variant thereof comprising at least 95% sequence identity to SEQ ID NO: 8);
h) the AX or AA genotype for the Single Nucleotide Polymorphism marker SNP_09 at nucleotide 101 of SEQ ID NO: 9 (or at nucleotide 101 in a variant thereof comprising at least 95% sequence identity to SEQ ID NO: 9).

In another aspect a L. sativa plant is provided comprising an introgression fragment on chromosome 7 in homozygous or heterozygous form, wherein said introgression fragment confers Fol-4 and/or Fol-1 resistance (compared to the plant lacking the introgression fragment, e.g. the recurrent parent or control) and wherein said introgression fragment comprises the SNP marker haplotype or genotype of at least 1, 2, 3, 4, 5, 6, 7, 8, 9 or 10 of the SNP markers selected from the group consisting of:
a) the AX or AA genotype for the Single Nucleotide Polymorphism marker SNP_01 at nucleotide 101 of SEQ ID NO: 1 (or at nucleotide 101 in a variant thereof comprising at least 95% sequence identity to SEQ ID NO: 1);
b) the GX or GG genotype for the Single Nucleotide Polymorphism marker SNP_02 at nucleotide 101 of SEQ ID NO: 2 (or at nucleotide 101 in a variant thereof comprising at least 95% sequence identity to SEQ ID NO: 2);
c) the CX or CC genotype for the Single Nucleotide Polymorphism marker SNP_03 at nucleotide 101 of SEQ ID NO: 3 (or at nucleotide 101 in a variant thereof comprising at least 95% sequence identity to SEQ ID NO: 3);
d) the AX or AA genotype for the Single Nucleotide Polymorphism marker SNP_04 at nucleotide 101 of SEQ ID NO: 4 (or at nucleotide 101 in a variant thereof comprising at least 95% sequence identity to SEQ ID NO: 4);
e) the GX or GG genotype for the Single Nucleotide Polymorphism marker SNP_05 at nucleotide 101 of SEQ ID NO: 5 (or at nucleotide 101 in a variant thereof comprising at least 95% sequence identity to SEQ ID NO: 5);
f) the GX or GG genotype for the Single Nucleotide Polymorphism marker SNP_06 at nucleotide 101 of SEQ ID NO: 6 (or at nucleotide 101 in a variant thereof comprising at least 95% sequence identity to SEQ ID NO: 6);
g) the TX or TT genotype for the Single Nucleotide Polymorphism marker SNP_07 at nucleotide 101 of SEQ ID NO: 7 (or at nucleotide 101 in a variant thereof comprising at least 95% sequence identity to SEQ ID NO: 7);
h) the TX or TT genotype for the Single Nucleotide Polymorphism marker SNP_08 at nucleotide 101 of SEQ ID NO: 8 (or at nucleotide 101 in a variant thereof comprising at least 95% sequence identity to SEQ ID NO: 8);
i) the AX or AA genotype for the Single Nucleotide Polymorphism marker SNP_09 at nucleotide 101 of SEQ ID NO: 9 (or at nucleotide 101 in a variant thereof comprising at least 95% sequence identity to SEQ ID NO: 9);
j) the GX or GG genotype for the Single Nucleotide Polymorphism marker SNP_10 at nucleotide 101 of SEQ ID NO: 10 (or at nucleotide 101 in a variant thereof comprising at least 95% sequence identity to SEQ ID NO: 10).

When referring to a SNP in a variant sequence, that variant sequence comprises at least 95%, 96%, 97%, 98% or 99% sequence identity with the mentioned sequence. X refers to any nucleotide for the sequence on the other chromosome 7 of the pair of chromosomes. In one aspect X may be the nucleotide of the recurrent parent as described in Table 1, 2 and 3. In another aspect X is a different nucleotide than the L. serriola donor nucleotide described in Tables 1, 2 and 3.

In one aspect said at least 1, 2, 3, 4, 5, 6, 7, 8, 9 or 10 markers are consecutive markers.

In one aspect the introgression fragment comprises the donor SNP marker haplotype or genotype for at least SNP_06, SNP_07 and SNP_08, as SNP_07 was mapped to be the peak marker for QTL7.1 (or a variant).

The fragment comprising the QTL7.1 (or variant) may, thus, be large (comprising the donor nucleotide for SNP_01 to SNP_10 or for SNP_02 to SNP_09), or may be smaller and lack markers having the genotype or haplotype of the L. serriola donor (e.g. the markers have the L. sativa genotype or haplotype instead, see also Table 1), but it may still confer Fol-4 and/or Fol-1 resistance onto the L. sativa plant, i.e. it can still comprise the QTL7.1 or a variant of QTL7.1. Such smaller introgression fragments are an embodiment of the invention. Plants having smaller introgression fragments which still confer the Fol-4 and/or Fol-1 resistance (i.e. contain the resistance conferring QTL7.1, or a variant) can be generated using known techniques, such as fine-mapping or similar techniques. For example by starting with a plant comprising the introgression fragment as found in seeds deposited under accession number NCIMB44108 and crossing such a plant with another L. sativa plant and selfing the progeny of said cross, and/or backcrossing the progeny, to generate a population of plants which may contain recombinants having a smaller introgression fragment on chromosome 7, which fragments still confer Fol-4 and/or Fol-1 resistance in relation to a plant lacking the introgression fragment (such as the control), e.g. a fragment comprising the donor nucleotide for markers SNP_03 to SNP_09, or SNP_04 to SNP_09 or SNP_05 to SNP_09 or SNP_05 to SNP_08 or SNP_6 to SNP_09, or SNP_06 to SNP_08.

Marker assays can be used to determine the size of the smaller introgression fragment. One or more of the SNP markers with the genotype or haplotype of the donor may be missing. The L. sativa genotype or haplotype is then detected for these SNP markers. The Fol-4 and/or Fol-1 resistance of plants comprising such a smaller introgression fragment can then be compared in a disease assay as described herein, i.e. growing a plurality of plants comprising the smaller introgression fragment in experiments together with suitable control plants, lacking the introgression fragments.

Alternatively, the same or variant QTL (QTL7.1 or variant QTL7.1) may be introgressed from a different L. serriola donor accession, whereby optionally not all SNP markers disclosed herein may be present, i.e. the SNP haplotype of the donor accession may only be identical to the SNP haplotype of the QTL7.1 present in seeds of NCIMB44108 for e.g. 2, 3, 4, 5, 6, 7 or 8 of the SNPs. Such alternative donor sources can be identified using the SNP markers provided herein, by screening germplasm (i.e. accessions of) L. serriola using a marker assay to detect the genotype or haplotype of one or more of markers SNP_01 to SNP_10, or e.g. of markers SNP_02 to SNP_09, SNP_03 to SNP_09, SNP_05 to SNP_09, or SNP_06 to SNP_09, or SNP_06 to SNP_08, or even only a smaller subgroup of these markers. Plants comprising the same or variant QTL7.1 from other L. serriola accessions are, therefore, also an embodiment of the invention. Thus, as long as at least 2, 3, 4, 5, 6 or 7, 8, 9 or 10 of the donor SNP nucleotides of SNP_01 to SNP_10, or of the SNPs of SNP_02 to SNP_09, SNP_03 to SNP_09, SNP_05 to SNP_09, or SNP_06 to SNP_09, or SNP_06 to SNP_08 are present, the donor may contain QTL7.1 (or a variant thereof) and is encompassed herein. The skilled person can then introgress the QTL7.1 (or a variant thereof) into L. sativa in order to enhance Fol-4 and/or Fol-1 resistance as described herein and in order to confirm that the QTL enhances Fol-4 and/or Fol-1 resistance when present in L. sativa. Prior to introgression the wild donor may also be tested for Fol-4 and/or Fol-1 resistance in an assay as described and e.g. a donor may be selected that comprises at least IR or HR resistance against Fol-4 and/or Fol-1.

As described above, in one embodiment the L. sativa plant of the invention comprises an introgression fragment comprising at least a subset of SNP markers with the genotype (or haplotype) of the L. serriola donor, i.e. at least 2, 3, 4, 5, 6, 7, 8, 9 or 10 markers of SNP_01 to SNP_10, or at least 2 or 3 or 4, 5, 6, 7 or 8 markers of SNP_01 to SNP_10, or of the SNPs of SNP_02 to SNP_09, SNP_03 to SNP_09, SNP_05 to SNP_09, or SNP_06 to SNP_09, or SNP_06 to SNP_08. In one aspect the cultivated lettuce plant comprises all, or all except 1 or 2 markers of SNP_01 to SNP_10, or all except 1 or 2 markers of SNP_02 to SNP_09. Especially one or more markers next to the flanking markers may be absent, e.g. SNP_02, or SNP_02 and SNP_03 or SNP_09 or SNP_09 and SNP 08.

Thus, the introgression fragment (and a L. sativa plant or plant part, e.g., a cell, comprising the introgression fragment) can be detected in a marker assay by detecting the SNP genotype or haplotype of the introgression fragment (i.e. of the donor genome fragment) of one or more or all of the markers above, preferably at least 2, 3, 4, 5, 6, 7, 8, 9 or 10.

Thus, in one aspect, a Quantitative Trait Locus (QTL7.1 or a variant thereof) was found to be present on chromosome 7 of a wild L. serriola donor which, when transferred (introgressed) into a L. sativa variety or breeding line, and when present in heterozygous or homozygous form, confers Fol-4 and/or Fol-1 resistance onto the cultivated lettuce plant, preferably dual resistance. The QTL, or the introgression fragment comprising the QTL, is additive, i.e. it is sufficient to have the introgression fragment on one of the chromosomes 7 (one recombinant chromosome 7), while the homologous chromosome 7 of the pair may be a (non-recombinant) chromosome 7 of L. sativa lacking the introgression fragment.

Accessions of L. serriola, are obtainable from e.g. the USDA National Plant Germplasm System collection or other seed collections, and can thus be screened for the presence of QTL7.1 (or a variant) using e.g. a marker assay as described herein, and accessions comprising 2, 3, 4, 5, 6, 7, 8 or more of the SNP markers indicative of QTL7.1 (or a variant) can be crossed with a L. sativa plant having normal wild-type, non-recombinant chromosomes 7. The F1 or F2 generation (or further generation, such as the F3 or a backcross generation) can then be screened for recombinant plants having the introgression fragment or a part thereof retaining the QTL, using the molecular marker assays described herein.

In one embodiment the introgression fragment comprising QTL7.1 (or a variant) is identifiable (and thus comprises) by one or more of the markers described elsewhere herein, especially the donor nucleotide for one or more of markers SNP_01 to SNP_10 or of SNP_02 to SNP_09 for the introgression fragment on chromosome 7, or a subset of markers. In one aspect the invention provides a L. sativa plant, having a genome of cultivated (domesticated) lettuce which comprises Fol-4 and/or Fol-1 resistance, wherein the resistance is conferred by an introgression fragment on the lettuce chromosome 7, wherein said introgression fragment, or wherein said QTL, is obtained by (or obtainable by) crossing a plant grown from seeds deposited under NCIMB 44108 or progeny of this plant (which comprises one or more the markers disclosed herein linked to the QTL) with another lettuce plant. Thus, in one aspect the L. sativa plant of the invention comprises the same introgression fragment and the same recombinant chromosome 7 as present in NCIMB 44108 (comprising all of the wild donor haplotype for SNP markers SNP_01 to SNP_10, or SNP_01 to SNP_09, or comprising SEQ ID NO: 1 to 10, or SEQ ID NO: 2 to 9, or SEQ ID NO: 11 to 20, or SEQ ID NO: 12 to 19), or it comprises a shorter fragment of that introgression fragment, whereby the shorter fragment retains the genetic element conferring resistance (QTL7.1).

Thus in one aspect the invention relates to a plant of the invention i.e. a cultivated L. sativa plant comprising an introgression fragment comprising QTL7.1 from a L. serriola plant, on chromosome 7, in homozygous or heterozygous form and wherein said introgression fragment is the introgression fragment “as in”/is “identical to”/is “the same as in” the seeds deposited under number NCIMB 44108, or is a shorter fragment (sub-fragment) thereof, but still confers Fol-4 and/or Fol-1 resistance due to the presence of QTL7.1 on the sub-fragment.

Likewise in one aspect the invention relates to a plant of the invention i.e. a cultivated L. sativa plant comprising an introgression fragment comprising QTL7.1 from a L. serriola plant, on chromosome 7, in homozygous or heterozygous form and wherein said QTL is the QTL “as in”/is “identical to”/is “the same as in” the seeds deposited under number NCIMB 44108 and confers Fol-4 and/or Fol-1 resistance due to the presence of QTL7.1.

As SEQ ID NO: 11 to 20 are from the wild donor used to generate NCIMB44108, one or more of these sequences, especially one or more of SEQ ID NO: 12 to 19 (or a sequence comprising at least 95%, 96%, 97%, 98% sequence identity to any one of SEQ ID NO: 11 to SEQ ID NO: 20) can be used to identify the introgression fragment or sub-fragments of the specific donor, as the entire sequence (the SNP nucleotide and the flanking sequences) is from the donor accession used in the Examples.

However, likewise one or more of the consensus sequences of Table 1 (SEQ ID NO: 1 to SEQ ID NO: 10, especially SEQ ID NO: 2 to SEQ ID NO: 9, or a sequence comprising at least 95%, 96%, 97%, 98% sequence identity to any one of these sequences) or the sequences of Table 3 can be used, as the donor SNP nucleotide of all these sequences is specific for L. serriola and the donor SNP nucleotide of SNP_02 to SNP_06 and of SNP_08 to SNP_09 is specific for the donor accession used in the Examples.

In one embodiment the invention relates to a plant of the invention i.e. a cultivated Lactuca sativa plant comprising an introgression fragment comprising QTL7.1 (or a variant) from a L. serriola donor on chromosome 7 in homozygous or heterozygous form and wherein said introgression fragment is the introgression fragment as in the deposited seeds, or is a variant of the introgression fragment of seeds deposited under number NCIMB 44108, i.e. it comprises the QTL 7.1 (or a variant), but the genomic sequence may be different. As wild accessions will be genetically divergent, the genomic sequence of an introgression fragment comprising QTL7.1 (or a variant) from other Fol-4 and/or Fol-1 resistant L. serriola accessions will most likely not be 100% identical to the genomic sequence as introgressed into NCIMB 44108, but e.g. at least 95%, 96%, 97% or 98% identical to the sequence. The divergence can be seen in that certain SNP markers linked to QTL7.1 (or a variant thereof) may be commonly found in various accessions, while other SNP markers may only be found in specific accessions. So, for example not all of SNP_2 to SNP_09 may be found in other L. serriola donors and the SNP donor haplotype may comprise 2, 3, 4, 5, 6, 7, 8 or 9 of the donor SNP markers SNP_1 to SNP_10, or of SNP_02 to SNP_09. For example, another Fol-4 and/or Fol-1 resistant L. serriola donor may have a slightly different donor SNP haplotype for SNP_01 to SNP_10, or for SNP_02 to SNP_09, with e.g. 1, 2 or 3 SNPs having a different donor nucleotide.

However, QTL7.1 (or a variant of QTL7.1) may still be present in such wild accessions. The skilled person is capable of identifying and introgressing the QTL7.1 (or variant) comprising region, as delimited by flanking markers of SNP_01 and SNP_10, found in other L. serriola donors into cultivated lettuce, e.g. detecting wild accessions comprising the SNP markers or a subset thereof and transferring these SNP markers (or subset) into a cultivated lettuce line or variety and assessing the Fol-4 and/or Fol-1 resistance of the cultivated line or variety compared to the line or variety lacking the SNP markers (or subset), i.e. lacking the introgression fragment.

Even in cases where the SNP haplotype for SNP_01 to SNP_10 or for SNP_02 to SNP_09 is identical to the SNP haplotype of QTL7.1 found in seeds of NCIMB 44108, the actual nucleotide sequences flanking the SNP nucleotide may be different in other donors and therefore also different in the consensus sequence which takes variation between the donor and the L. sativa reference genome into account. So other donors may comprise the same donor SNP nucleotide at nucleotide 101, but in a sequence comprising at least 95%, 96%, 97%, 98% or 99% sequence identity to SEQ ID NO: 1 to (or to SEQ ID NO: 2 to 9) when e.g. aligned pairwise, or in a sequence comprising at least 95%, 96%, 97%, 98% or 99% sequence identity to SEQ ID NO: 11 to 20 (or to SEQ ID NO: 12 to 19) when aligned pairwise, or in a sequence comprising at least 95%, 96%, 97%, 98% or 99% sequence identity to SEQ ID NO: 21 to 30 (or to SEQ ID NO: 22 to 29) when aligned pairwise.

In one embodiment the presence of the introgression fragment comprising QTL7.1, or the chromosome 7 region (or variant or orthologous chromosome 7 region), comprising QTL7.1, is detectable by (and the genome comprises one or more molecular markers) a molecular marker assay which detects at least 1, 2, 3, 4, 5, 6, 7, 8, 9 or 10 Single Nucleotide Polymorphism (SNP) markers selected from the group consisting of:
a) the AX or AA genotype for the Single Nucleotide Polymorphism marker SNP_01 at nucleotide 101 of SEQ ID NO: 1, or of SEQ ID NO: 11, or of SEQ ID NO: 21 (or at nucleotide 101 in a variant thereof comprising at least 95%, 96%, 97%, 98% or 99% sequence identity to SEQ ID NO: 1, or SEQ ID NO: 11 or SEQ ID NO: 21); (this marker is optional, as it is a flanking marker, which delimits the QTL comprising region at one side)
b) the GX or GG genotype for the Single Nucleotide Polymorphism marker SNP_02 at nucleotide 101 of SEQ ID NO: 2, or of SEQ ID NO: 12, or of SEQ ID NO: 22 (or at nucleotide 101 in a variant thereof comprising at least 95%, 96%, 97%, 98% or 99% sequence identity to SEQ ID NO: 2, or SEQ ID NO: 12 or SEQ ID NO: 22);
c) the CX or CC genotype for the Single Nucleotide Polymorphism marker SNP_03 at nucleotide 101 of SEQ ID NO: 3, or of SEQ ID NO: 13, or of SEQ ID NO: 23 (or at nucleotide 101 in a variant thereof comprising at least 95%, 96%, 97%, 98% or 99% sequence identity to SEQ ID NO: 3 or SEQ ID NO: 13 or SEQ ID NO: 23);
d) the AX or AA genotype for the Single Nucleotide Polymorphism marker SNP_04 at nucleotide 101 of SEQ ID NO: 4, or of SEQ ID NO: 14, or of SEQ ID NO: 24 (or at nucleotide 101 in a variant thereof comprising at least 95%, 96%, 97%, 98% or 99% sequence identity to SEQ ID NO: 4 or SEQ ID NO: 14 or SEQ ID NO: 24);
e) the GX or GG genotype for the Single Nucleotide Polymorphism marker SNP_05 at nucleotide 101 of SEQ ID NO: 5, or of SEQ ID NO: 15, or of SEQ ID NO: 25 (or at nucleotide 101 in a variant thereof comprising at least 95%, 96%, 97%, 98% or 99% sequence identity to SEQ ID NO: 5 or SEQ ID NO: 15 or SEQ ID NO: 25);
f) the GX or GG genotype for the Single Nucleotide Polymorphism marker SNP_06 at nucleotide 101 of SEQ ID NO: 6, or of SEQ ID NO: 16, or of SEQ ID NO: 26 (or at nucleotide 101 in a variant thereof comprising at least 95%, 96%, 97%, 98% or 99% sequence identity to SEQ ID NO: 6 or SEQ ID NO: 16 or SEQ ID NO: 26);
g) the TX or TT genotype for the Single Nucleotide Polymorphism marker SNP_07 at nucleotide 101 of SEQ ID NO: 7, or of SEQ ID NO: 17, or of SEQ ID NO: 27 (or at nucleotide 101 in a variant thereof comprising at least 95%, 96%, 97%, 98% or 99% sequence identity to SEQ ID NO: 7 or SEQ ID NO: 17 or SEQ ID NO: 27);
h) the TX or TT genotype for the Single Nucleotide Polymorphism marker SNP_08 at nucleotide 101 of SEQ ID NO: 8, or of SEQ ID NO: 18, or of SEQ ID NO: 28 (or at nucleotide 101 in a variant thereof comprising at least 95%, 96%, 97%, 98% or 99% sequence identity to SEQ ID NO: 8 or SEQ ID NO: 18 or SEQ ID NO: 28);
i) the AX or AA genotype for the Single Nucleotide Polymorphism marker SNP_09 at nucleotide 101 of SEQ ID NO: 9, or of SEQ ID NO: 19, or of SEQ ID NO: 29 (or at nucleotide 101 in a variant thereof comprising at least 95%, 96%, 97%, 98% or 99% sequence identity to SEQ ID NO: 9 or SEQ ID NO: 19 or SEQ ID NO: 29);
j) the GX or GG genotype for the Single Nucleotide Polymorphism marker SNP_10 at nucleotide 101 of SEQ ID NO: 10, or of SEQ ID NO: 20, or of SEQ ID NO: 30 (or at nucleotide 101 in a variant thereof comprising at least 95%, 96%, 97%, 98% or 99% sequence identity to SEQ ID NO: 10 or SEQ ID NO: 20 or SEQ ID NO: 30); (this marker is optional, as it is a flanking marker, which delimits the QTL comprising region at the other side).

Thus, in one embodiment the presence of the introgression fragment comprising QTL7.1 (or a variant), or the chromosome 7 region (or variant chromosome 7 region), comprising QTL7.1 (or a variant), is detectable by (and the genome comprises one or more molecular markers) a molecular marker assay which detects at least 1, 2, 3, 4, 5, 6, 7, or 8 Single Nucleotide Polymorphism (SNP) markers selected from the group consisting of:
a) the GX or GG genotype for the Single Nucleotide Polymorphism marker SNP_02 at nucleotide 101 of SEQ ID NO: 2, or of SEQ ID NO: 12, or of SEQ ID NO: 22 (or at nucleotide 101 in a variant thereof comprising at least 95%, 96%, 97%, 98% or 99% sequence identity to SEQ ID NO: 2, or SEQ ID NO: 12 or SEQ ID NO: 22);
b) the CX or CC genotype for the Single Nucleotide Polymorphism marker SNP_03 at nucleotide 101 of SEQ ID NO: 3, or of SEQ ID NO: 13, or of SEQ ID NO: 23 (or at nucleotide 101 in a variant thereof comprising at least 95%, 96%, 97%, 98% or 99% sequence identity to SEQ ID NO: 3 or SEQ ID NO: 13 or SEQ ID NO: 23);
c) the AX or AA genotype for the Single Nucleotide Polymorphism marker SNP_04 at nucleotide 101 of SEQ ID NO: 4, or of SEQ ID NO: 14, or of SEQ ID NO: 24 (or at nucleotide 101 in a variant thereof comprising at least 95%, 96%, 97%, 98% or 99% sequence identity to SEQ ID NO: 4 or SEQ ID NO: 14 or SEQ ID NO: 24);
d) the GX or GG genotype for the Single Nucleotide Polymorphism marker SNP_05 at nucleotide 101 of SEQ ID NO: 5, or of SEQ ID NO: 15, or of SEQ ID NO: 25 (or at nucleotide 101 in a variant thereof comprising at least 95%, 96%, 97%, 98% or 99% sequence identity to SEQ ID NO: 5 or SEQ ID NO: 15 or SEQ ID NO: 25);
e) the GX or GG genotype for the Single Nucleotide Polymorphism marker SNP_06 at nucleotide 101 of SEQ ID NO: 6, or of SEQ ID NO: 16, or of SEQ ID NO: 26 (or at nucleotide 101 in a variant thereof comprising at least 95%, 96%, 97%, 98% or 99% sequence identity to SEQ ID NO: 6 or SEQ ID NO: 16 or SEQ ID NO: 26);
f) the TX or TT genotype for the Single Nucleotide Polymorphism marker SNP_07 at nucleotide 101 of SEQ ID NO: 7, or of SEQ ID NO: 17, or of SEQ ID NO: 27 (or at nucleotide 101 in a variant thereof comprising at least 95%, 96%, 97%, 98% or 99% sequence identity to SEQ ID NO: 7 or SEQ ID NO: 17 or SEQ ID NO: 27);
g) the TX or TT genotype for the Single Nucleotide Polymorphism marker SNP_08 at nucleotide 101 of SEQ ID NO: 8, or of SEQ ID NO: 18, or of SEQ ID NO: 28 (or at nucleotide 101 in a variant thereof comprising at least 95%, 96%, 97%, 98% or 99% sequence identity to SEQ ID NO: 8 or SEQ ID NO: 18 or SEQ ID NO: 28);
h) the AX or AA genotype for the Single Nucleotide Polymorphism marker SNP_09 at nucleotide 101 of SEQ ID NO: 9, or of SEQ ID NO: 19, or of SEQ ID NO: 29 (or at nucleotide 101 in a variant thereof comprising at least 95%, 96%, 97%, 98% or 99% sequence identity to SEQ ID NO: 9 or SEQ ID NO: 19 or SEQ ID NO: 29).

In one aspect said at least 1, 2, 3, 4 or more markers which are detected are consecutive markers.

In one aspect at least SNP_07 is detected (i.e. is present in the genome), in addition to one or more markers, especially one or more markers consecutive with SNP_07, e.g. SNP_06 and/or SNP_08, or SNP_05 and SNP_06 and/or SNP_08, or SNP_08 and/or SNP_09. Thus, in one aspect SNP_07 is present/detected and in addition one or more consecutive markers selected from upstream and/or downstream of SNP_07, such as SNP_2 to SNP_06 upstream and SNP_08 and SNP_09 downstream of SNP_07, see FIG. 4 also.

Thus, in one embodiment the lettuce plant, seed or tissue according to the invention comprises
optionally an Adenine (A) (i.e. the AA or AX genotype) at nucleotide 101 of SEQ ID NO: 1, or of SEQ ID NO: 11, or of SEQ ID NO: 21 (referred to as SNP_01) or at the equivalent nucleotide of a genomic sequence comprising substantial sequence identity to SEQ ID NO:

1, 11 or 21 (in other words there is an Adenine at the physical position of chromosome 7 shown in Table 1);

and/or
at least a Guanine (G) (i.e. the GG or GX genotype) at nucleotide 101 of SEQ ID NO: 2, or of SEQ ID NO: 12 or of SEQ ID NO: 22 (referred to as SNP_02) or at the equivalent nucleotide of a genomic sequence comprising substantial sequence identity to SEQ ID NO: 2, 12 or 22 (in other words there is a Guanine at the physical position of chromosome 7 shown in Table 1);
   and/or
at least a Cytosine (C) (i.e. the CC or CX genotype) at nucleotide 101 of SEQ ID NO: 3, or of SEQ ID NO: 13 or of SEQ ID NO: 23 (referred to as SNP_03) or at the equivalent nucleotide of a genomic sequence comprising substantial sequence identity to SEQ ID NO: 3, 13 or 23 (in other words there is a Cytosine at the physical position of chromosome 7 shown in Table 1);
   and/or
at least an Adenine (A) (i.e. the AA or AX genotype) at nucleotide 101 of SEQ ID NO: 4, or of SEQ ID NO: 14 or of SEQ ID NO: 24 (referred to as SNP_04) or at the equivalent nucleotide of a genomic sequence comprising substantial sequence identity to SEQ ID NO: 4, 14 or 24 (in other words there is an Adenine at the physical position of chromosome 7 shown in Table 1);
   and/or
at least a Guanine (G) (i.e. the GG or GX genotype) at nucleotide 101 of SEQ ID NO: 5, or of SEQ ID NO: 15 or of SEQ ID NO: 25 (referred to as SNP_05) or at the equivalent nucleotide of a genomic sequence comprising substantial sequence identity to SEQ ID NO: 5, 15 or 25 (in other words there is a Guanine at the physical position of chromosome 7 shown in Table 1);
   and/or
at least a Guanine (G) (i.e. the GG or GX genotype) at nucleotide 101 of SEQ ID NO: 6, or of SEQ ID NO: 16 or of SEQ ID NO: 26 (referred to as SNP_06) or at the equivalent nucleotide of a genomic sequence comprising substantial sequence identity to SEQ ID NO: 6, 16 or 26 (in other words there is a Guanine at the physical position of chromosome 7 shown in Table 1);
   and/or
at least a Thymine (T) (i.e. the TT or TX genotype) at nucleotide 101 of SEQ ID NO: 7, or of SEQ ID NO: 17 or of SEQ ID NO: 27 (referred to as SNP_07) or at the equivalent nucleotide of a genomic sequence comprising substantial sequence identity to SEQ ID NO: 7, 17 or 27 (in other words there is a Thymine at the physical position of chromosome 7 shown in Table 1);
   and/or
at least a Thymine (T) (i.e. the TT or TX genotype) at nucleotide 101 of SEQ ID NO: 8, or of SEQ ID NO: 18 or of SEQ ID NO: 28 (referred to as SNP_08) or at the equivalent nucleotide of a genomic sequence comprising substantial sequence identity to SEQ ID NO: 8, 18 or 28 (in other words there is a Thymine at the physical position of chromosome 7 shown in Table 1);
   and/or
at least an Adenine (A) (i.e. the AA or AX genotype) at nucleotide 101 of SEQ ID NO: 9, or of SEQ ID NO: 19 or of SEQ ID NO: 29 (referred to as SNP_09) or at the equivalent nucleotide of a genomic sequence comprising substantial sequence identity to SEQ ID NO: 9, 19 or 29 (in other words there is an Adenine at the physical position of chromosome 7 shown in Table 1);
   and/or
optionally a Guanine (G) (i.e. the GG or GX genotype) at nucleotide 101 of SEQ ID NO: 10, or of SEQ ID NO: 20 or of SEQ ID NO: 30 (referred to as SNP_10) or at the equivalent nucleotide of a genomic sequence comprising substantial sequence identity to SEQ ID NO: 10, 20 or 30 (in other words there is an Guanine at the physical position of chromosome 7 shown in Table 1).

In a further one embodiment the presence of the introgression fragment, or the chromosome 7 region (or variant or orthologous chromosome 7 region), comprising QTL7.1, is detectable by a molecular marker assay which detects at least 3, 4 or 5 Single Nucleotide Polymorphism (SNP) markers of the sub-groups consisting of: SNP_06 to SNP_08; SNP_04 to SNP_08; SNP_05 to SNP_08, SNP_04 to SNP_09, SNP_04 to SNP_08, SNP_03 to SNP_09, SNP_03 to SNP_08, SNP_02 to SNP_09, SNP_02 to SNP_08, SNP_05 to SNP_07, or SNP_07 to SNP_09.

The SNP genotype refers to two nucleotides, and genomic sequences comprising one of these two nucleotides, one on each chromosome 7. So a plant having a TT genotype for SNP_07 has an identical nucleotide (T) on both chromosomes (i.e. is homozygous), while a plant having an TX genotype for SNP_07 has one chromosome with an T at nucleotide 101 of SEQ ID NO: 1, 11 or 21 (or at the equivalent nucleotide of a genomic sequence comprising substantial sequence identity to SEQ ID NO:1, 11 or 21) and one chromosome with a X at nucleotide 101 of SEQ ID NO: 1, 11 or 21 (or at the equivalent nucleotide of a genomic sequence comprising substantial sequence identity to SEQ ID NO:1, 11 or 21) and is heterozygous, whereby X may be any nucleotide. As the genomic sequences around the SNP markers provided herein may vary slightly in introgression fragments from other donors (i.e. variants or orthologous chromosome 7 regions) it is clear that the nucleotide sequences before and after the SNP may not be 100% identical to the sequences provided herein. Therefore, sequences having substantial sequence identity (i.e. at least 95% identity) to the sequences provided herein, but which comprise the same SNP nucleotide at the SNP position, are encompassed herein.

In one aspect, the introgression fragment comprising QTL7.1 (or a variant), or the chromosome 7 region (or variant or orthologous chromosome 7 region) comprising the QTL (QTL7.1 or variant), which is detectable by the above one or more markers, is from a L. serriola accession which is resistant to Fol-4 and/or Fol-1. In one aspect it is the same introgression fragment as found on chromosome 7 in seeds deposited under accession number NCIMB 44108, or a smaller fragment retaining the QTL. SNP markers SNP_01 to SNP_10 span a region of about 5 Mb on chromosome 7. In one aspect the introgression fragment on chromosome 7 is equal to or less than 5 Mb in size, preferably equal to or less than 4.9 Mb in size, more preferably equal to or less than 4.5 Mb or 4.0 Mb in size, e.g. equal to or less than 3 Mb or 2 Mb or 1.5 Mb. In one aspect the introgression fragment is at least 0.2 Mb, 0.5 Mb, 1.0 Mb, 1.1 Mb, 1.5 Mb, 1.9 Mb, 2.0 Mb, 2.5 Mb, 2.7 Mb or 3 Mb in size. Thus, various ranges of introgression sizes are encompassed herein, such as fragments less than 5 Mb but more than 0.2 Mb, less than 4 Mb or 3 Mb but more than 0.2 Mb, 0.5 Mb, 1 Mb or 1.1 Mb, etc., which retain the QTL7.1 (or a variant) and one or more of the SNP markers of SNP_01 to SNP_10 or one or more of the SNP markers of SNP_02 to SNP_09, or of the subgroups described above. As mentioned before, the location of the QTL7.1 (or a variant) in the region spanning SNP_01 to SNP_10 can be determined by finemapping and recombinants comprising QTL7.1 (or a variant) on a smaller introgression fragment can be generated. The size of an introgression fragment can be easily determined by e.g. whole genome sequencing or Next Generation Sequencing. Especially introgression regions can be easily distinguished from cultivated genomic regions due to the genetic variation (SNPs, INDELs, etc.) in the introgression region.

To obtain the introgression fragment present on chromosome 7 (comprising QTL7.1) from the deposited seeds (NCIMB44108), i.e. to transfer the introgression fragments comprising the QTL to another cultivated lettuce plant, a plant is grown from the seed and the plant is crossed with a cultivated lettuce plant to obtain F1 seeds. As NCIMB44108 contains two recombinant chromosomes 7 (comprising the introgression fragment) all of the F1 seed and plants grown therefrom, contain one recombinant chromosome 7 from the NCIMB44108 parent and one non-recombinant chromosome 7 from the other cultivated parent. Thus, by traditional breeding one can transfer the recombinant chromosome 7 from NCIMB44108 into other cultivated lettuce lines or varieties. Plants which comprise the QTL7.1 can be screened for, and selected for, by the presence of one or more of the above SNP markers in order to identify plants comprising a recombinant chromosome 7.

To generate shorter introgression fragments (comprising QTL7.1) meiosis needs to take place and plants comprising the recombinant chromosomes 7, and especially new meiotic recombination events within the introgression fragment, need to be identified. For example, seeds of NCIMB44108 can be selfed one or more times to produce S1, S2 or S3 plants (or further selfing generations), and/or S1, S2 or S3 plants (etc.) comprising a recombinant chromosome 7 can be backcrossed to a cultivated parent. Plants which comprise the recombinant chromosome 7 can be screened for, and selected for, by the presence of one or more of the above SNP markers in order to identify plants comprising a smaller introgression fragment. Such new recombinants can then be tested for the presence of the QTL7.1 on the smaller introgression fragment by determining the average disease score in a Fol-4 and/or Fol-1 disease assay compared to the (genetic) control lacking the introgression fragment.

Similarly, cultivated lettuce plants comprising QTL7.1 (or a variant thereof) can be generated and/or identified using different methods. For example, to obtain a cultivated lettuce plant comprising an introgression fragment from a wild L. serriola donor, a wild L. serriola donor is identified which comprises one or more of the SNP markers linked to QTL7.1 (or a variant) disclosed herein, e.g. any one, or more, or all of the markers described herein above. This can, for example, be done for a large number of accessions. The identified plant is crossed with a cultivated lettuce plant to obtain F1 seeds. The F1 can be selfed to produce F2, F3, etc. plants, and/or F2 plants or F3 plants, etc., can be backcrossed to the cultivated lettuce parent. Plants which are comprising QTL7.1 (or a variant thereof) can be screened for, and/or selected for, by the presence of one or more of the above SNP markers and/or screened for and/or selected for Fol-4 and/or Fol-1 resistance phenotype compared to the initial cultivated parent (lacking the introgressions). Alternatively or in addition, QTL mapping can be carried out in order to identify further molecular markers linked to the QTL7.1 (or a variant thereof) and/or to generate cultivated lettuce plants comprising an introgression fragment on chromosome 7 which confers Fol-4 and/or Fol-1 resistance.

In one embodiment the presence of the introgression fragment in a cultivated lettuce plant, or the chromosome 7 region (or variant chromosome 7 region), comprising QTL7.1, is detectable by a molecular marker assay which detects at least 3, 4, 5, 6, 7 or 8 of the markers selected from the group consisting of:
a) the GG or GX genotype for the Single Nucleotide Polymorphism marker SNP_02 in SEQ ID NO: 2 (or in a variant thereof);
b) the AA or AX genotype for the Single Nucleotide Polymorphism marker SNP_09 in SEQ ID NO: 9 (or in a variant thereof);
c) any L. serriola—genome specific marker in between marker SNP_02 and SNP_09.

In one aspect the markers of c) are one or more of SNP_03 to SNP_08. In one aspect, at least 3, 4, 5, 6, 7 or 8 markers are detected from the markers of a), b) and/or c) above. In one embodiment at least the marker of a) and/or b) is detected and optionally at least one, two, three or more markers of c) are detected. In one aspect the at least one marker of c) comprises SNP_07. In one aspect the markers detected are consecutive markers.

Any L. serriola—genome specific marker in between two markers refers to any molecular marker which maps genetically to the chromosome 7 region in-between the two markers and/or which lies physically in-between the two markers, and which is indicative of the L. serriola chromosome 7 region. This means that the marker is polymorphic between the cultivated L. sativa genome and the wild L. serriola genome. In one aspect, the marker is a Single Nucleotide Polymorphism (SNP), but other molecular markers such as INDELs, RFLP, AFLP, RAPD, DNA sequencing, etc. may equally be used.

The introgression fragment in the plants of the invention is, in one aspect, a fragment of chromosome 7 (comprising QTL7.1) which is present in seeds deposited under accession number NCIMB44108 or a smaller version of that fragment retaining the QTL (generated by e.g. recombination within the introgression fragment).

Also provided are seeds from which a plant of the invention can be grown, as are heads or leaves harvested from a plant of the invention and comprising the recombinant chromosome 7 in their genome (comprising QTL7.1 or a variant). Likewise, a plant cell, tissue or plant part of a plant or of a seed is provided comprising at least one recombinant chromosome 7 (comprising QTL7.1 or a variant), wherein said recombinant chromosome 7 comprises an introgression fragment from a L. serriola accession and wherein said introgression fragment comprises a QTL conferring Fol-4 and/or Fol-1 resistance, e.g. HR or IR resistance to either race. In one aspect the L. serriola donor and the QTL7.1 from the donor confers at least IR or HR resistance against race Fol-4 and optionally further also IR or HR resistance against race Fol-1.

The molecular markers described herein may be detected according to standard method. For example SNP markers can easily be detected using a KASP-assay (see www.kpbioscience.co.uk) or other SNP genotyping assays. For developing a KASP-assay, for example 50 or 70 base pairs upstream and 50 or 70 base pairs downstream of the SNP can be selected and two allele-specific forward primers and one allele specific reverse primer can be designed. See e.g. Allen et al. 2011, Plant Biotechnology J. 9, 1086-1099, especially p097-1098 for KASP assay method.

Thus, in one aspect, the SNP markers and the presence/absence of the marker associated with QTL7.1 is determined using a KASP assay, but equally other SNP genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

The physical size of an introgression fragment can be determined by various methods, such as physical mapping, sequencing or by visualization of the introgression using Fluorescent in situ hybridization (FISH) images (Verlaan et al. 2011, Plant Journal 68:1093-1103).

Cultivated lettuce plants with smaller introgression fragments on chromosome 7 (comprising QTL7.1 or a variant) can be generated by generating new recombinant plants from a population of plants derived from a cross between a cultivated lettuce plant (lacking the introgressions) and a plant of the invention and selecting recombinant progeny having smaller introgression sizes. Such plants are, thus, in one aspect derived from (progeny or descendants of) the recombinant chromosome 7 present in plants of which seeds have been deposited under NCIMB44108. Such progeny or descendants which retain the QTL7.1, and thus the higher Fol-4 and/or Fol-1 resistance compared to plants lacking an introgression as described herein, are encompassed herein.

In one aspect the plants and plant parts which comprise QTL7.1 (or a variant thereof) comprise an introgression fragment comprising the QTL that is derivable from or obtainable from (or is derived from, or obtained from) NCIMB 44108 or from a L. serriola donor which comprises the same SNP haplotype or SNP genotype for at least 3, 4, 5, 6, 7, 8, 9 or 10 of the SNP markers linked to QTL7.1. The wild donor comprises at least Fol-4 resistance, but optionally further also Fol-1 resistance, i.e. dual resistance.

The lettuce plant, seed, plant part, cell or tissue comprising QTL7.1 (or a variant) may be any type of lettuce plant, e.g. baby leaf, Romaine, iceberg, etc., suitable for e.g. open field or indoor cultivation. The Lactuca sativa plant according to the invention may be a heading and non-heading type of lettuce. In one aspect, the Lactuca sativa plant according to the present invention is of the head lettuce type or of the teen leaf type, loose leaf type or baby leaf type.

As the QTL7.1 (or a variant) has an additive effect, the lettuce plant, seed, plant part, cell or tissue preferably comprises QTL7.1 (or a variant) in homozygous form.

When referring to a lettuce plant herein, also seeds from which such a plant can be grown are encompassed, as are plant parts (e.g. heads, leaves, leaf parts, etc.), tissues and cells.

Preferably the plant has good agronomic quality characteristics. The plant is in one aspect uniform, both genetically and phenotypically.

In one aspect the lettuce plant is the plant of which seeds were deposited under accession number NCIMB 441108, or progeny thereof, whereby the progeny retain QTL7.1 (as detectable by the presence of one or more markers as described elsewhere).

A further embodiment of the invention is a plant cell, tissue or plant part of a plant or of a seed according to the invention comprising at least one recombinant chromosome 7 wherein said recombinant chromosome 7 comprises an introgression fragment which comprises QTL7.1 (or a variant) as described.

In one aspect, the Lactuca sativa plant according to the present invention, comprising QTL7.1 from L. serriola, is an inbred plant, a dihaploid plant or a hybrid plant. In one aspect, accordingly, the present invention provides that the plant of the present invention is an inbred plant. Such an inbred plant is highly homozygous, for instance by repeated selfing or self pollination steps. In one aspect, the disclosure provides for haploid plants and/or dihaploid (double haploid) plants of plant of the invention are encompassed herein, which comprise the QTL7.1 (or variant) as described herein.

Haploid and dihaploid plants can for example be produced by anther or microspore culture and regeneration into a whole plant. For dihaploid production chromosome doubling may be induced using known methods, such as colchicine treatment or the like. So, in one aspect a Lactuca sativa plant is provided, comprising QTL7.1 (or variant) as described, wherein the plant is a dihaploid plant.

The plants provided by the present invention may be used to produce food or feed. The present invention, thus, provides the use of a plant of the species Lactuca sativa, as provided herein, as a crop for consumption. Particularly the harvested leaves and/or harvested heads produced by the plants of the present invention can be advantageously used as a crop for consumption.

The plants provided by the present invention may be used to produce propagation material. Such propagation material comprises propagation material suitable for and/or resulting from sexual reproduction, such as pollen and seeds. Such propagation material comprises propagation material suitable for and/or resulting from asexual or vegetative reproduction including, but not limited to cuttings, grafts, tubers, cell culture and tissue culture. The present invention, thus, further provides the use of a Lactuca sativa plant as provided herein as a source of propagation material.

In one aspect, the present invention provides seed from which the Lactuca sativa plant according to the present invention can be grown. Furthermore, the invention provides a plurality of such seed. A seed of the invention can be distinguished from other seeds due to the presence of the QTL7.1 (or a variant, or the introgression fragment comprising QTL7.1 or a variant) as described herein, either phenotypically (based on plants having a Fol-4 and/or Fol-1 resistance phenotype of the present invention) and/or using molecular methods to detect the SNP markers linked to the QTL, or the SNP markers indicative of the introgression fragment, in the cells or tissues, such as molecular genotyping methods to detect the introgression fragment of the present invention, or sequencing.

Also, a method for identifying (detecting or selecting) a cultivated L. sativa plant or plant part comprising an introgression fragment on chromosome 7 comprising QTL7.1 (or a variant thereof) is provided, wherein said introgression fragment or said QTL7.1 (or a variant thereof), is e.g. as found in NCIMB 44108, or in a L. serriola donor, comprising:
a) providing a cultivated L. sativa plant or plant part or DNA of such plant or plant part,
b) screening said plant, plant part or DNA using a molecular marker assay which detects at least 1, 2, 3, 4, 5, 6, 7, 8, 9 or 10 SNP markers of SNP_01 to SNP_10, or of SNP_02 to SNP_09, for detecting the introgression fragment on chromosome 7 comprising QTL7.1 (or a variant); and
c) identifying and/or selecting a plant comprising the donor SNP nucleotide (the donor SNP haplotype or donor SNP genotype) for at least 1, 2, 3, 4, 5, 6, 7, 8, 9 or 10 of SNP_01 to SNP_10, or of SNP_02 to SNP_09, for detecting the introgression fragment on chromosome 7 comprising QTL7.1 (or a variant).

Further a method of producing an L. sativa plant comprising an introgression fragment on chromosome 7 comprising QTL7.1 (or a variant thereof) comprising:
a) providing a first lettuce (L. sativa) plant comprising a recombinant chromosome 7 in homozygous form having an introgression fragment comprising QTL7.1 (or a variant thereof) and comprising the donor SNP nucleotide (the donor SNP haplotype or donor SNP genotype) for at least 1, 2, 3, 4, 5, 6, 7, 8, 9 or 10 of the SNP markers linked to the QTL, optionally wherein said introgression fragment or said QTL7.1 is derivable from (or derived from) NCIMB 44108,
b) providing a second lettuce (L. sativa) plant,
c) crossing said lettuce plant of a) with said lettuce plant of b),
d) collecting seeds from said cross.

The seeds collected are also an embodiment of the invention.

In another aspect a method for generating progeny of NCIMB 44108 is provided, said method comprising:
a) growing a plant from seeds deposited under accession number NCIMB 44108;
b) selfing said plant one or more times and/or crossing said plant one or more times with another L. sativa plant to generate progeny seeds;
c) screening said progeny seeds or plants grown from said seeds or parts of the seeds or plants using a molecular marker assay which detects at least 1, 2, 3, 4, 5, 6, 7, 8, 9, 10 or more SNP markers of:
   i) SNP_01 to SNP_10, or SNP_02 to SNP_09, for detecting the introgression fragment on chromosome 7 comprising QTL7.1 (or a variant); and/or
d) identifying and/or selecting a progeny plant comprising the donor SNP nucleotide (the donor SNP haplotype or donor SNP genotype) for at least 1, 2, 3, 4, 5, 6, 7, 8, 9 or 10 of SNP_01 to SNP_10, or SNP_02 to SNP_09, for detecting the introgression fragment on chromosome 7 comprising QTL7.1.

The donor SNP nucleotide (or haplotype or genotype) is described above and in Tables 1 to 3.

Further provided is a method for identifying and/or selecting a wild L. serriola donor plant comprising QTL7.1 (or a variant thereof), comprising:
a) screening seeds or parts of the seeds or plants or parts of plants or DNA of such plants, seeds or plant parts of one or more L. serriola accessions using a molecular marker assay which detects at least 1, 2, 3, 4, 5, 6, 7, 8, 9 or 10 SNP markers of:
   i) SNP_01 to SNP_10, or SNP_02 to SNP_09, for detecting the introgression fragment on chromosome 7 comprising QTL7.1 (or a variant);
b) identifying and/or selecting a L. serriola accession comprising the donor SNP nucleotide (the donor SNP haplotype or donor SNP genotype) for at least 1, 2, 3, 4, 5, 6, 7, 8, 9 or 10 of SNP_01 to SNP_10, or of SNP_02 to SNP_09, for detecting the introgression fragment on chromosome 7 comprising QTL7.1 (or a variant).

The above method may optionally also comprise selfing the wild L. serriola accessions one or more times, e.g. prior to step a).

Wild accessions may be e.g. from seed depositories, such as USDA ARS-GRIN collections, CGN collections, and others.

The method above may also include a step of testing the one or more L serriola accessions in a Fol-4 and/or Fol-1 resistance assay. This may e.g. be done prior to the molecular marker assay of step a), prior to selfing the accession(s) or after selfing the accession(s) and/or after step b), i.e. after selection or identification of one or more accessions comprising a SNP haplotype or SNP genotype identical or similar to the one described in Tables 1 to 3, e.g. the SNP haplotype for QTL7.1 (or a variant) having the donor nucleotide for at least 1, 2, 3, 4, 5, 6, 7, 8, 9 or 10 of the SNP markers linked to the QTL7.1 (or a variant thereof). The one or more L. serriola accessions may be selected for accessions (or selfing progenies thereof) having an average Fol-4 and/or Fol-1 disease score of 2.0 or less, preferably of 1.5 or less, e.g. 1.0 or less than 1.0, using the protocol described e.g. in the Examples, or the one or more L. serriola accessions may be selected for accessions having a significantly increased Fol-4 and/or Fol-1 resistance compared to the susceptible control variety or varieties included in the disease assay.

Also a method for transferring QTL7.1 (or a variant thereof) from a L. serriola donor into a L. sativa plant is provided comprising:
a) providing a L. serriola accession (or a progeny thereof obtained by selfing one or more times) comprising the donor SNP nucleotide (the donor SNP haplotype or donor SNP genotype) for at least 1, 2, 3, 4, 5, 6, 7, 8, 9 or 10 of
   i) SNP_01 to SNP_10, or SNP_02 to SNP_09, for detecting the introgression fragment on chromosome 7 comprising QTL7.1 (or a variant), and optionally comprising an average Fol-4 and/or Fol-1 disease score of 2.0 or less, preferably of 1.5 or less, e.g. 1.0 or less than 1.0; or optionally comprising significantly increased Fol-4 and/or Fol-1 resistance compared to the control;
b) crossing said accession with a L. sativa plant to obtain progenies of the F1, F2, F3 or further selfing generations or BC1, BC2, BC3 or further backcross generations, and optionally
c) selecting a progeny plant comprising QTL7.1 (or a variant) from the L. serriola accession of step a).

A progeny plant generated by the above method is also an aspect of the invention.

Also, containers and packages containing or comprising seeds from which plants of the invention can be grown are provided herein. These may be labelled as containing L. sativa seeds producing plants having Fusarium wilt resistance, at least against races Fol-4 and/or Fol-1.

Also, progeny seeds and progeny plants of plants of the invention are provided, which retain the introgression on chromosome 7 comprising QTL7.1 (or a variant), or which comprise a smaller introgression fragment comprising QTL7.1 or a variant QTL7.1 (e.g. derivable from the fragment as is present in NCIMB 44108) which still confers Fol-4 and/or Fol-1 resistance, i.e. which still contains the QTL. Progeny may be any generation obtained by selfing a lettuce plant according to the invention and/or crossing a lettuce plant according to the invention with another lettuce plant one or more times.

Progeny are, therefore, either the generation (seeds) produced from the first cross (F1) or selfing (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another L. sativa plant. Progeny are preferably selected to retain the introgression fragment from L. serriola comprising QTL7.1 (or a variant thereof). Thus, progeny also have the increased Fol-4 and/or Fol-1 resistance phenotype, preferably at least the same average disease score as the plant used in the initial cross or selfing. The presence of (or retention of) the introgression fragment comprising the QTL can be determined phenotypically and/or using the molecular marker assay(s) described herein. Regarding phenotypic assessment, of course consideration needs to be given to the additive nature of the QTL.

In a further aspect parts of the L. sativa plants according to the invention are provided. Parts include for example cells and cell-cultures, tissue cultures, vegetative plant tissues (leaves, roots, etc.), flowers, pollen, embryos, etc. The plant parts comprise the introgression fragment on chromosome 7, as described, and as can be detected using one or more of the markers described. Also, when whole plants are regenerated from such parts, such as cells, cell- or tissue cultures, the regenerated plants comprise the recombinant chromosome 7.

Thus, also provided is a plant cell, tissue or plant part of a plant or of a seed according the invention comprising at least one recombinant chromosome 7, wherein said recombinant chromosome 7 comprises an introgression fragment from a L. serriola donor plant and wherein said introgression fragment comprises a QTL conferring Fol-4 and/or Fol-1 resistance, selected from QTL7.1 or a variant thereof.

Also in vitro cell cultures and in vitro tissue cultures are encompassed herein, of cells or tissues comprising a recombinant chromosome 7 described. Preferably the cells or tissues can be regenerated into a whole lettuce plant, i.e. the cells are regenerable cells and the tissues comprise regenerable cells. Thus, also vegetative propagations of the plants according to the invention are an embodiment herein. Thus, a vegetatively propagated L. sativa plant is provided which comprises a recombinant chromosome 7 as described herein. In a different aspect non-propagating cells comprising a QTL7.1 (or a variant thereof) conferring Fol-4 and/or Fol-1 resistance,) are encompassed herein, as are tissues comprising such cells.

In a specific aspect a head or leaf harvested from a plant according to the invention is provided.

Also, containers or packages comprising or consisting of harvested heads or leaves are provided. Again, the cells of the heads or leaves are distinguishable from other heads or leaves by the presence of the QTL7.1 (or a variant thereof) (as determinable in one or more of the molecular marker assays).

The invention also provides for a food or feed product comprising or consisting of a plant part described herein preferably a head or leaves described herein. The food or feed product may be fresh or processed, e.g., pickled, canned, steamed, boiled, fried, blanched and/or frozen, etc. For example, containers such as cans, boxes, crates, bags, cartons, Modified Atmosphere Packaging, films (e.g. biodegradable films), etc. comprising plant parts such as heads or leaves described herein are also provided herein.

### Methods and Uses According to the Invention

In a further embodiment, the invention provides for a method of producing a new cultivated lettuce plant which comprises an introgression fragment on chromosome 7, which comprises QTL7.1, or a variant thereof, and wherein said QTL confers Fol-4 and/or Fol-1 resistance compared to a control plant, in homozygous or heterozygous form, as described. The method comprises crossing a plant of the invention, or a progeny plant thereof, either as male or as female parent, with a second lettuce plant one or more times, and/or selfing a lettuce plant according to the invention, or a progeny plant thereof, one or more times, and selecting progeny from said crossing and/or selfing.

Thus, a method for transferring the recombinant chromosome 7, comprising QTL7.1 or a variant thereof from one (cultivated) lettuce plant into another (cultivated) clettuce plant is provided, especially into lettuce varieties or breeding lines for which the Fusarium Fol-4 and/or Fol-1 resistance should be introduced or increased.

The method comprises the steps of:
a) providing a first cultivate lettuce plant comprising an introgression fragment comprising QTL7.1 (or a variant thereof), preferably in homozygous form,
b) providing a second cultivated lettuce plant, especially a plant having a wild type (non-recombinant) chromosome 7, or a plant lacking QTL7.1,
c) crossing said lettuce plant of a) with said lettuce plant of b),
d) collecting F1 hybrid seeds from said cross, and
e) optionally selfing the plant grown from said F1 hybrid seeds to produce F2 seeds or further selfing generations and/or backcrossing the seeds to produce backcross generations, and optionally selecting the F2 seeds or further selfing generation seeds or backcross generation seeds having the QTL7.1 (or a variant thereof), and
f) optionally breeding further with plants grown from said F1 or F2 or further generation selfing seeds or backcross generation seeds to produce a lettuce plant having good agronomic characteristics and QTL7.1 (or a variant thereof) in homozygous or heterozygous form.

The presence or absence of the introgression fragment comprising QTL7.1 (or a variant thereof) may be determined by one or more of the molecular marker assays described herein and/or by determining whether the Fol-4 and/or Fol-1 resistance is significantly increased compared to the plant of e.g. step b). Further breeding in step e) or f) may comprise selfing, crossing, double haploid production, backcrossing, and combinations thereof (e.g. backcrossing and selfing), etc. Plants, plant parts and seeds obtainable by the above method are encompassed herein.

In one aspect the plant of step a) may be a plant grown from seeds deposited under NCIMB 44108, or progeny thereof, or a plant comprising QTL7.1 (or a variant thereof), as e.g. present in other wild L. serriola donor accession comprising the same SNP haplotype or genotype for at least 2, 3, 4, 5, 6, 7, 8, 9 or 10 markers linked to the QTL (e.g. as described in Tables 1 to 3).

In a different aspect a method for producing a cultivated lettuce plant comprising an introgression fragment on chromosome 7, wherein said introgression fragment comprises QTL7.1 or a variant thereof, is provided, said method comprising the steps:
a) providing a first cultivated lettuce (L. sativa) plant,
b) providing a second wild L. serriola plant, wherein said plant comprises QTL7.1 or a variant thereof, as determinable by the presence of one or more SNP markers (especially as determinable by the presence of the donor SNP haplotype or genotype for at least 2, 3, 4, 5, 6, 7, 8, 9 or 10 SNP markers linked to the QTL) as described herein,
c) crossing said lettuce plant of a) with said lettuce plant of b),
d) collecting F1 seeds from said cross and backcrossing an F1 plant to the lettuce plant of a) to produce a backcross (BC1) population, or selfing said F1 plants one or more times to produce an F2 or F3 or higher generation selfing population,
e) optionally backcrossing a plant of d) one or more times to the lettuce plant of a) to produce a higher generation backcross population, and
f) identifying a F2, F3, or higher generation selfing, or BC1 or higher generation backcross plant which comprises an introgression on chromosome 7, wherein said introgression fragment comprises QTL7.1 or a variant thereof.

When referring to backcross populations in the method, the backcross populations may also be selfed, i.e. BC1S1, BC1S2, BC2S1, BC2S2, or others.

In one or more of steps b) to f) the presence of the QTL (or the introgression fragment comprising the QTL) may be tested (and plants may be selected) by carrying out a molecular marker assay as described elsewhere herein, e.g. by determining whether the plant comprises the one or more of the SNP markers (e.g. one or more of SNP_01 to SNP_10, or SNP_02 to SNP_09) linked to QTL7.1 and/or testing the disease resistance against Fol-4 and/or Fol-1.

Using this method, one can generate and/or select new cultivated lettuce plants comprising an introgression with QTL7.1, or a variant thereof, from a L. serriola donor. In one aspect the L. serriola in step b) is selected from a donor comprising at least 2, 3, 4, 5, 6, 7, 8, 9 or 10 of the SNP markers (SNP haplotype or SNP genotype) linked to the QTL (see e.g. Tables 1 to 3) and comprising Fol-4 and/or Fol-1 resistance, e.g. an average Fol-4 disease score of equal to or below 2.0, 1.5 or 1.0, or significantly enhanced Fol-4 and/or Fol-1 resistance compared to a susceptible control plant.

In one aspect the method for producing a cultivated lettuce plant comprising an introgression fragment on chromosome 7, wherein said introgression fragment comprises QTL7.1 or a variant thereof, comprises the steps:
a) providing a first cultivated lettuce plant,
b) providing a second wild L. serriola comprising at least 2, 3, 4, 5, 6, 7, 8, 9 or 10 of the SNP markers (SNP haplotype or SNP genotype) linked to QTL7.1, and optionally comprising an average Fol-4 and/or Fol-1 disease score of equal to or below 2.0, 1.5 or 1.0 or optionally comprising significantly increased resistance against Fol-4 and/or Fol-1 compared to the control,
c) crossing said plant of a) with said plant of b),
d) collecting F1 seeds from said cross and backcrossing an F1 plant to the plant of a) to produce a backcross (BC1) population, or selfing said F1 plants one or more times to produce an F2 or F3 population,
e) optionally selfing the backcross population to produce e.g. a BC1S1 or BC1S2 population,
f) identifying a F2, F3, BC1, BC1S1, or BC1S2 plant which comprises the (one or more) SNP markers.

Also provided is a method for identifying a wild L. serriola plant comprising a Fol-4 and/or Fol-1 resistance QTL on chromosome 7, said method comprising:
A) providing a L. serriola accession or several accessions;
B) screening said accession(s) using a molecular marker assay which detects at least 1, 2, 3, 4, 5, 6, 7, 8, 9 or 10 of the SNP markers linked to QTL7.1;
C) identifying and/or selecting an accession from b) comprising the SNP haplotype or SNP genotype of at least 2, 3, 4, 5, 6, 7, 8, 9 or 10 of the SNP markers linked to a QTL, selected from:
a) the AX or AA genotype for the Single Nucleotide Polymorphism marker SNP_01 at nucleotide 101 of SEQ ID NO: 1, or of SEQ ID NO: 11, or of SEQ ID NO: 21 (or at nucleotide 101 in a variant thereof comprising at least 95%, 96%, 97%, 98% or 99% sequence identity to SEQ ID NO: 1, or SEQ ID NO: 11 or SEQ ID NO: 21); (this marker is optional, as it is a flanking marker, which delimits the QTL-comprising region on one side)
b) the GX or GG genotype for the Single Nucleotide Polymorphism marker SNP_02 at nucleotide 101 of SEQ ID NO: 2, or of SEQ ID NO: 12, or of SEQ ID NO: 22 (or at nucleotide 101 in a variant thereof comprising at least 95%, 96%, 97%, 98% or 99% sequence identity to SEQ ID NO: 2, or SEQ ID NO: 12 or SEQ ID NO: 22);
c) the CX or CC genotype for the Single Nucleotide Polymorphism marker SNP_03 at nucleotide 101 of SEQ ID NO: 3, or of SEQ ID NO: 13, or of SEQ ID NO: 23 (or at nucleotide 101 in a variant thereof comprising at least 95%, 96%, 97%, 98% or 99% sequence identity to SEQ ID NO: 3 or SEQ ID NO: 13 or SEQ ID NO: 23);
d) the AX or AA genotype for the Single Nucleotide Polymorphism marker SNP_04 at nucleotide 101 of SEQ ID NO: 4, or of SEQ ID NO: 14, or of SEQ ID NO: 24 (or at nucleotide 101 in a variant thereof comprising at least 95%, 96%, 97%, 98% or 99% sequence identity to SEQ ID NO: 4 or SEQ ID NO: 14 or SEQ ID NO: 24);
e) the GX or GG genotype for the Single Nucleotide Polymorphism marker SNP_05 at nucleotide 101 of SEQ ID NO: 5, or of SEQ ID NO: 15, or of SEQ ID NO: 25 (or at nucleotide 101 in a variant thereof comprising at least 95%, 96%, 97%, 98% or 99% sequence identity to SEQ ID NO: 5 or SEQ ID NO: 15 or SEQ ID NO: 25);
f) the GX or GG genotype for the Single Nucleotide Polymorphism marker SNP_06 at nucleotide 101 of SEQ ID NO: 6, or of SEQ ID NO: 16, or of SEQ ID NO: 26 (or at nucleotide 101 in a variant thereof comprising at least 95%, 96%, 97%, 98% or 99% sequence identity to SEQ ID NO: 6 or SEQ ID NO: 16 or SEQ ID NO: 26);
g) the TX or TT genotype for the Single Nucleotide Polymorphism marker SNP_07 at nucleotide 101 of SEQ ID NO: 7, or of SEQ ID NO: 17, or of SEQ ID NO: 27 (or at nucleotide 101 in a variant thereof comprising at least 95%, 96%, 97%, 98% or 99% sequence identity to SEQ ID NO: 7 or SEQ ID NO: 17 or SEQ ID NO: 27);
h) the TX or TT genotype for the Single Nucleotide Polymorphism marker SNP_08 at nucleotide 101 of SEQ ID NO: 8, or of SEQ ID NO: 18, or of SEQ ID NO: 28 (or at nucleotide 101 in a variant thereof comprising at least 95%, 96%, 97%, 98% or 99% sequence identity to SEQ ID NO: 8 or SEQ ID NO: 18 or SEQ ID NO: 28);
i) the AX or AA genotype for the Single Nucleotide Polymorphism marker SNP_09 at nucleotide 101 of SEQ ID NO: 9, or of SEQ ID NO: 19, or of SEQ ID NO: 29 (or at nucleotide 101 in a variant thereof comprising at least 95%, 96%, 97%, 98% or 99% sequence identity to SEQ ID NO: 9 or SEQ ID NO: 19 or SEQ ID NO: 29);
j) the GX or GG genotype for the Single Nucleotide Polymorphism marker SNP_10 at nucleotide 101 of SEQ ID NO: 10, or of SEQ ID NO: 20, or of SEQ ID NO: 30 (or at nucleotide 101 in a variant thereof comprising at least 95%, 96%, 97%, 98% or 99% sequence identity to SEQ ID NO: 10 or SEQ ID NO: 20 or SEQ ID NO: 30); (this marker is optional, as it is a flanking marker, which delimits the QTL-comprising region on the other side),
D) optionally introgressing said QTL from said wild accession into cultivated lettuce (e.g. by backcrossing).

In step B), C) and D) a SNP genotyping assay can for example be used (such as a KASP assay), but also other molecular marker assays can be used. This applies also to other methods described herein. With this method one can, thus, screen wild L. serriola accessions for the presence of one or more of the markers linked to QTL7.1 and introgress the QTL into cultivated lettuce plants. Plants and seeds obtained by this method are also an embodiment of the invention.

Further provided is a method for identifying a wild L. serriola plant comprising a Fol-4 and/or Fol-1 resistance QTL on chromosome 7, said method comprising:
A) providing a L. serriola plant or a plurality of L. serriola plants;
B) optionally testing the L. serriola plant or the plurality of L. serriola plants for resistance against Fusarium races Fol-4 and/or Fol-1, e.g. in a disease assay;
C) screening the genomic DNA of the plant or plurality of plants of A), or optionally only the Fol-4 and/or Fol-1 resistant plant or plants identified in B), for the presence of one or more markers indicative of QTL7.1; and
D) identifying a plant and/or selecting a plant comprising one or more markers of C);
E) optionally testing the plant of D) for Fusarium race Fol-4 and/or Fol-1 resistance;
   wherein the markers indicative of QTL7.1 are selected from the group consisting of:
a) the AX or AA genotype for the Single Nucleotide Polymorphism marker SNP_01 at nucleotide 101 of SEQ ID NO: 1, or of SEQ ID NO: 11, or of SEQ ID NO: 21 (or at nucleotide 101 in a variant thereof comprising at least 95%, 96%, 97%, 98% or 99% sequence identity to SEQ ID NO: 1, or SEQ ID NO: 11 or SEQ ID NO: 21); (this marker is optional, as it is a flanking marker which delimits the QTL-comprising region on one side)
b) the GX or GG genotype for the Single Nucleotide Polymorphism marker SNP_02 at nucleotide 101 of SEQ ID NO: 2, or of SEQ ID NO: 12, or of SEQ ID NO: 22 (or at nucleotide 101 in a variant thereof comprising at least 95%, 96%, 97%, 98% or 99% sequence identity to SEQ ID NO: 2, or SEQ ID NO: 12 or SEQ ID NO: 22);
c) the CX or CC genotype for the Single Nucleotide Polymorphism marker SNP_03 at nucleotide 101 of SEQ ID NO: 3, or of SEQ ID NO: 13, or of SEQ ID NO: 23 (or at nucleotide 101 in a variant thereof comprising at least 95%, 96%, 97%, 98% or 99% sequence identity to SEQ ID NO: 3 or SEQ ID NO: 13 or SEQ ID NO: 23);
d) the AX or AA genotype for the Single Nucleotide Polymorphism marker SNP_04 at nucleotide 101 of SEQ ID NO: 4, or of SEQ ID NO: 14, or of SEQ ID NO: 24 (or at nucleotide 101 in a variant thereof comprising at least 95%, 96%, 97%, 98% or 99% sequence identity to SEQ ID NO: 4 or SEQ ID NO: 14 or SEQ ID NO: 24);
e) the GX or GG genotype for the Single Nucleotide Polymorphism marker SNP_05 at nucleotide 101 of SEQ ID NO: 5, or of SEQ ID NO: 15, or of SEQ ID NO: 25 (or at nucleotide 101 in a variant thereof comprising at least 95%, 96%, 97%, 98% or 99% sequence identity to SEQ ID NO: 5 or SEQ ID NO: 15 or SEQ ID NO: 25);
f) the GX or GG genotype for the Single Nucleotide Polymorphism marker SNP_06 at nucleotide 101 of SEQ ID NO: 6, or of SEQ ID NO: 16, or of SEQ ID NO: 26 (or at nucleotide 101 in a variant thereof comprising at least 95%, 96%, 97%, 98% or 99% sequence identity to SEQ ID NO: 6 or SEQ ID NO: 16 or SEQ ID NO: 26);
g) the TX or TT genotype for the Single Nucleotide Polymorphism marker SNP_07 at nucleotide 101 of SEQ ID NO: 7, or of SEQ ID NO: 17, or of SEQ ID NO: 27 (or at nucleotide 101 in a variant thereof comprising at least 95%, 96%, 97%, 98% or 99% sequence identity to SEQ ID NO: 7 or SEQ ID NO: 17 or SEQ ID NO: 27);
h) the TX or TT genotype for the Single Nucleotide Polymorphism marker SNP_08 at nucleotide 101 of SEQ ID NO: 8, or of SEQ ID NO: 18, or of SEQ ID NO: 28 (or at nucleotide 101 in a variant thereof comprising at least 95%, 96%, 97%, 98% or 99% sequence identity to SEQ ID NO: 8 or SEQ ID NO: 18 or SEQ ID NO: 28);
i) the AX or AA genotype for the Single Nucleotide Polymorphism marker SNP_09 at nucleotide 101 of SEQ ID NO: 9, or of SEQ ID NO: 19, or of SEQ ID NO: 29 (or at nucleotide 101 in a variant thereof comprising at least 95%, 96%, 97%, 98% or 99% sequence identity to SEQ ID NO: 9 or SEQ ID NO: 19 or SEQ ID NO: 29);
j) the GX or GG genotype for the Single Nucleotide Polymorphism marker SNP_10 at nucleotide 101 of SEQ ID NO: 10, or of SEQ ID NO: 20, or of SEQ ID NO: 30 (or at nucleotide 101 in a variant thereof comprising at least 95%, 96%, 97%, 98% or 99% sequence identity to SEQ ID NO: 10 or SEQ ID NO: 20 or SEQ ID NO: 30); (this marker is optional, as it is a flanking marker which delimits the QTL-comprising region on the other side).

In still another aspect a method for identifying a cultivated lettuce plant comprising an introgression fragment on chromosome 7, wherein said introgression fragment comprises a Fol-4 and/or Fol-1 resistance QTL, is provided, said method comprising: screening a cultivated lettuce plant or a population of cultivated lettuce plants or parts of such lettuce plants (e.g. cells, DNA) using a molecular marker assay which detects at least one SNP marker (preferably 2, 3, 4, 5 or more; preferably consecutive SNP markers) indicative of (linked to) QTL7.1 (or a variant thereof) as described elsewhere herein.

In this method any of the molecular marker tests described elsewhere herein can be used. Thus, using this method one can detect the presence of an introgression fragment comprising QTL7.1 (or a variant thereof) in cultivated lettuce plants or plant parts.

In yet another aspect a method for detecting whether a cultivated lettuce plant comprises an introgression fragment on chromosome 7, wherein said introgression fragment comprises QTL7.1 (or a variant thereof), is provided, said method comprising:
a) providing cultivated lettuce plant or a plant part,
b) screening said plant or said plant part (or DNA obtained from said plant or plant part) using a molecular marker assay which detects at least one (preferably at least 2, 3, 4, 5, 6, 7, 8, 9, or more) SNP marker selected from the group consisting of:
   SNP_01 to SNP_10 linked to QTL7.1 (or a variant); or SNP_02 to SNP_09 linked to QTL7.1 (or a variant).

Furthermore, provided is a method for screening cultivated lettuce lines or varieties for the presence of QTL7.1 comprising the steps of:
A) providing a cultivated lettuce plant or a plurality of cultivated lettuce plants;
B) optionally testing the cultivated lettuce plant or the plurality of cultivated lettuce plants for resistance against Fusarium races Fol-4 and/or Fol-1, e.g. in a disease assay;
C) screening the genomic DNA of the plant or plurality of plants of A), or optionally only the Fol-4 and/or Fol-1 resistant plant or plants identified in B), for the presence of one or more markers indicative of QTL7.1; and
D) identifying a plant and/or selecting a plant comprising one or more markers of C);
E) optionally testing the plant of D) for Fusarium race Fol-4 and/or Fol-1 resistance;
wherein the markers indicative of QTL7.1 are selected from the group consisting of:
a) the AX or AA genotype for the Single Nucleotide Polymorphism marker SNP_01 at nucleotide 101 of SEQ ID NO: 1, or of SEQ ID NO: 11, or of SEQ ID NO: 21 (or at nucleotide 101 in a variant thereof comprising at least 95%, 96%, 97%, 98% or 99% sequence identity to SEQ ID NO: 1, or SEQ ID NO: 11 or SEQ ID NO: 21); (this marker is optional, as it is a flanking marker)
b) the GX or GG genotype for the Single Nucleotide Polymorphism marker SNP_02 at nucleotide 101 of SEQ ID NO: 2, or of SEQ ID NO: 12, or of SEQ ID NO: 22 (or at nucleotide 101 in a variant thereof comprising at least 95%, 96%, 97%, 98% or 99% sequence identity to SEQ ID NO: 2, or SEQ ID NO: 12 or SEQ ID NO: 22);
c) the CX or CC genotype for the Single Nucleotide Polymorphism marker SNP_03 at nucleotide 101 of SEQ ID NO: 3, or of SEQ ID NO: 13, or of SEQ ID NO: 23 (or at nucleotide 101 in a variant thereof comprising at least 95%, 96%, 97%, 98% or 99% sequence identity to SEQ ID NO: 3 or SEQ ID NO: 13 or SEQ ID NO: 23);
d) the AX or AA genotype for the Single Nucleotide Polymorphism marker SNP_04 at nucleotide 101 of SEQ ID NO: 4, or of SEQ ID NO: 14, or of SEQ ID NO: 24 (or at nucleotide 101 in a variant thereof comprising at least 95%, 96%, 97%, 98% or 99% sequence identity to SEQ ID NO: 4 or SEQ ID NO: 14 or SEQ ID NO: 24);
e) the GX or GG genotype for the Single Nucleotide Polymorphism marker SNP_05 at nucleotide 101 of SEQ ID NO: 5, or of SEQ ID NO: 15, or of SEQ ID NO: 25 (or at nucleotide 101 in a variant thereof comprising at least 95%, 96%, 97%, 98% or 99% sequence identity to SEQ ID NO: 5 or SEQ ID NO: 15 or SEQ ID NO: 25);
f) the GX or GG genotype for the Single Nucleotide Polymorphism marker SNP_06 at nucleotide 101 of SEQ ID NO: 6, or of SEQ ID NO: 16, or of SEQ ID NO: 26 (or at nucleotide 101 in a variant thereof comprising at least 95%, 96%, 97%, 98% or 99% sequence identity to SEQ ID NO: 6 or SEQ ID NO: 16 or SEQ ID NO: 26);
g) the TX or TT genotype for the Single Nucleotide Polymorphism marker SNP_07 at nucleotide 101 of SEQ ID NO: 7, or of SEQ ID NO: 17, or of SEQ ID NO: 27 (or at nucleotide 101 in a variant thereof comprising at least 95%, 96%, 97%, 98% or 99% sequence identity to SEQ ID NO: 7 or SEQ ID NO: 17 or SEQ ID NO: 27);
h) the TX or TT genotype for the Single Nucleotide Polymorphism marker SNP_08 at nucleotide 101 of SEQ ID NO: 8, or of SEQ ID NO: 18, or of SEQ ID NO: 28 (or at nucleotide 101 in a variant thereof comprising at least 95%, 96%, 97%, 98% or 99% sequence identity to SEQ ID NO: 8 or SEQ ID NO: 18 or SEQ ID NO: 28);
i) the AX or AA genotype for the Single Nucleotide Polymorphism marker SNP_09 at nucleotide 101 of SEQ ID NO: 9, or of SEQ ID NO: 19, or of SEQ ID NO: 29 (or at nucleotide 101 in a variant thereof comprising at least 95%, 96%, 97%, 98% or 99% sequence identity to SEQ ID NO: 9 or SEQ ID NO: 19 or SEQ ID NO: 29);
j) the GX or GG genotype for the Single Nucleotide Polymorphism marker SNP_10 at nucleotide 101 of SEQ ID NO: 10, or of SEQ ID NO: 20, or of SEQ ID NO: 30 (or at nucleotide 101 in a variant thereof comprising at least 95%, 96%, 97%, 98% or 99% sequence identity to SEQ ID NO: 10 or SEQ ID NO: 20 or SEQ ID NO: 30); (this marker is optional, as it is a flanking marker).

Thus, in one aspect a method for detecting or selecting a, e.g. L. sativa or L. serriola, plant or plant part or seed comprising QTL7.1 is provided, said method comprising determining the presence in the genome of the plant, plant part or seed of at least 2, 3, 4, 5, 6, 7 or 8 of the following SNP markers:
a Guanine (G) for SNP_02 at nucleotide 101 of SEQ ID NO: 2 or at the equivalent nucleotide of a sequence comprising at least 95% sequence identity to SEQ ID NO: 2,
a Cytosine (C) for SNP_03 at nucleotide 101 of SEQ ID NO: 3 or at the equivalent nucleotide of a sequence comprising at least 95% sequence identity to SEQ ID NO: 3,
an Adenine (A) for SNP_04 at nucleotide 101 of SEQ ID NO: 4 or at the equivalent nucleotide of a sequence comprising at least 95% sequence identity to SEQ ID NO: 4,
a Guanine (G) for SNP_05 at nucleotide 101 of SEQ ID NO: 5 or at the equivalent nucleotide of a sequence comprising at least 95% sequence identity to SEQ ID NO: 5,
a Guanine (G) for SNP_06 at nucleotide 101 of SEQ ID NO: 6 or at the equivalent nucleotide of a sequence comprising at least 95% sequence identity to SEQ ID NO: 6,
a Thymine (T) for SNP_07 at nucleotide 101 of SEQ ID NO: 7 or at the equivalent nucleotide of a sequence comprising at least 95% sequence identity to SEQ ID NO: 7,
a Thymine (T) for SNP_08 at nucleotide 101 of SEQ ID NO: 8 or at the equivalent nucleotide of a sequence comprising at least 95% sequence identity to SEQ ID NO: 8,
an Adenine (A) for SNP_09 at nucleotide 101 of SEQ ID NO: 9 or at the equivalent nucleotide of a sequence comprising at least 95% sequence identity to SEQ ID NO: 9, and optionally selecting a plant, or plant part or seed comprising said SNP markers.

The method may further comprise the step of:
testing the plant or seed comprising said at least 2, 3, 4, 5, 6, 7 or 8 SNP markers for resistance against Fusarium race Fol-4 and/or Fol-1, and optionally selecting the plant or seed comprising resistance against Fusarium race Fol-4 and/or Fol-1.

Similarly, a method for detecting or selecting a, e.g. L. sativa or L. serriola, plant or plant part or seed comprising QTL7.1 is provided, comprising the steps of:
I) testing a plant or seed (or a plurality of plants or seeds) for resistance against Fusarium race Fol-4 and/or Fol-1; and
II) determining the presence in the genome of the plant, plant part or seed of at least 2, 3, 4, 5, 6, 7 or 8 of the following SNP markers:
   a Guanine (G) for SNP_02 at nucleotide 101 of SEQ ID NO: 2 or at the equivalent nucleotide of a sequence comprising at least 95% sequence identity to SEQ ID NO: 2,
   a Cytosine (C) for SNP_03 at nucleotide 101 of SEQ ID NO: 3 or at the equivalent nucleotide of a sequence comprising at least 95% sequence identity to SEQ ID NO: 3,
   an Adenine (A) for SNP_04 at nucleotide 101 of SEQ ID NO: 4 or at the equivalent nucleotide of a sequence comprising at least 95% sequence identity to SEQ ID NO: 4,
   a Guanine (G) for SNP_05 at nucleotide 101 of SEQ ID NO: 5 or at the equivalent nucleotide of a sequence comprising at least 95% sequence identity to SEQ ID NO: 5,
   a Guanine (G) for SNP_06 at nucleotide 101 of SEQ ID NO: 6 or at the equivalent nucleotide of a sequence comprising at least 95% sequence identity to SEQ ID NO: 6,
   a Thymine (T) for SNP_07 at nucleotide 101 of SEQ ID NO: 7 or at the equivalent nucleotide of a sequence comprising at least 95% sequence identity to SEQ ID NO: 7,
   a Thymine (T) for SNP_08 at nucleotide 101 of SEQ ID NO: 8 or at the equivalent nucleotide of a sequence comprising at least 95% sequence identity to SEQ ID NO: 8,
   an Adenine (A) for SNP_09 at nucleotide 101 of SEQ ID NO: 9 or at the equivalent nucleotide of a sequence comprising at least 95% sequence identity to SEQ ID NO: 9,
      and optionally
III) selecting a plant, or plant part or seed comprising Fol-4 and/or Fol-1 resistance and at least 2, 3, 4, 5, 6, 7 or 8 of said SNP markers.

Steps I and II) may be changed in order. So, one may first determine the presence of one or more donor SNP markers linked to QTL7.1 and indicative of QTL7.1 in the genome, and then test the plant line or seed (i.e. plant grown from the seed) for the resistance phenotype conferred by QTL7.1 or a variant thereof.

Molecular marker screening obviously involves obtaining plant material and analyzing the genomic DNA of the material for the SNP marker haplotype or genotype.

In the above methods also other molecular marker tests described elsewhere herein can be used.

Also encompassed herein is a method for producing a cultivated lettuce plant comprising an introgression fragment on chromosome 7, wherein said introgression fragments comprise QTL7.1 (or a variant thereof), comprising:
a) providing a first cultivated lettuce plant, preferably lacking QTL7.1,
b) providing a second cultivated lettuce plant selected from plants grown from seeds deposited under accession number NCIMB44108 or progeny thereof, or providing a second cultivated lettuce plant or wild L. serriola plant comprising QTL7.1 (or a variant thereof) and comprising a resistant donor SNP haplotype or genotype for at least 2, 3, 4, 5, 6, 7, 8, 9 or SNP markers linked to the QTL,
c) crossing said plant of a) with said plant of b),
d) collecting F1 seeds from said cross and optionally selfing said F1 plants one or more times to produce an F2 or F3 or further selfing population,
e) optionally backcrossing the F1 plant or an F2 or F3 or further selfing plant to the plant of a) to produce a backcross population,
f) optionally selfing the backcross population one or more times,
g) identifying a F1, F2, F3, further selfing or backcross plant which comprises the resistant donor SNP haplotype or genotype for at least 2, 3, 4, 5, 6, 7, 8, 9 or 10 SNP markers linked to the QTL.

In another aspect a method for generating progeny of NCIMB44108 retaining QTL7.1 is provided, said method comprising:
a) growing a plant from seeds deposited under accession number NCIMB 44108;
b) selfing said plant one or more times or crossing said plant one or more times with another cultivated lettuce plant to generate progeny seeds;
c) screening said progeny seeds or plants grown from said seeds or parts of the seeds or plants using a molecular marker assay which detects at least 1, 2, 3, 4, 5, 6, 7, 8, 9 or 10 SNP marker disclosed herein as being linked to QTL7.1;
d) identifying and/or selecting a progeny plant comprising a resistant donor SNP haplotype or genotype for at least 2, 3, 4, 5, 6, 7, 8, 9 or 10 SNP markers linked to the QTL (as described elsewhere herein); and
e) optionally confirming the Fol-4 and/or Fol-1 resistance of said progeny plants.

A method for generating progeny of NCIMB 44108 is provided, said method comprising:
a) growing a plant from seeds deposited under accession number NCIMB 44108;
b) selfing said plant one or more times or crossing said plant one or more times with another lettuce plant to generate progeny seeds;
c) screening said progeny seeds or plants grown from said seeds or parts of the seeds or plants using a molecular marker assay which detects at least 1, 2, 3, 4, 5, 6, 7, 8, 9 or SNP marker linked to QTL7.1, wherein the SNP markers are:
   SNP_01 to SNP_10 for detecting the introgression fragment comprising QTL7.1; or
   SNP_02 to SNP_09 for detecting the introgression fragment comprising QTL7.1;
d) identifying and/or selecting a progeny plant comprising:
   i) at least 2, 3, 4, 5, 6, 7, 8, 9 or 10 markers of SNP_01 to SNP_10 which have the resistant donor SNP haplotype; and/or
   ii) at least 2, 3, 4, 5, 6, 7, 8, 9 or 10 markers of SNP_02 to SNP_09 which have the resistant donor SNP haplotype; and/or
   iii) at least SNP_07 and further 1, 2, or 3 markers flanking SNP_07, selected from SNP_04, SNP_05, SNP_06, SNP_08, SNP_09 which have the resistant donor SNP haplotype;
e) optionally confirming the Fol-4 and/or Fol-1 resistance of said progeny plants.

A progeny plant generated by any of the above methods is also an aspect of the invention.

One can also use the methods and the markers described herein to reduce the size of the introgression fragment comprising the QTL, i.e. to generate and select recombinants having a smaller introgression fragment, but which retain the QTL.

In one aspect the invention encompasses the use of a recombinant chromosome 7 comprising an introgression fragment from a L. serriola accession, said introgression fragment comprising QTL7.1 (or a variant thereof), for breeding lettuce varieties having Fol-4 and/or Fol-1 resistance, especially dual resistance.

Also provided is the use of a chromosome 7 as found in seeds deposited under accession number NCIMB 44108, or progeny thereof, for generating a cultivated lettuce plant comprising an introgression fragment of said chromosome 7.

Also provided is the use of plants grown from seeds deposited under accession number NCIMB 44108, or progeny thereof, for generating a cultivated lettuce plant comprising Fol-4 and/or Fol-1 resistance, wherein said resistance is conferred by an introgression fragment obtained from chromosome 7 of said plants or progeny.

When referring to QTL7.1 as present in the deposited seeds (NCIMB 44108) or progeny thereof, it is noted that sequence flanking the SNP at nucleotide 101 provided in Table 2 is the flanking sequence of the specific donor used in the mapping and introgression. The SNP marker at nucleotide 101 may, however, also be present in a sequence comprising less than 100% sequence identity to the sequence provided under SEQ ID NO: 11 to 20, e.g. at nucleotide 101 of a sequence comprising at least 95%, 96%, 97%, 98%, 99% sequence identity to the provided sequence. Sequence identity of the region flanking the SNP can e.g. be analyzed by BLAST analysis or by pairwise alignment of sequences of the same length comprising the SNP at nucleotide 101 (using e.g. Needle, with default parameters). In the same way, Table 1 shows the SNP at nucleotide 101 in a sequence wherein the flanking sequences upstream and downstream of the SNP nucleotide are consensus sequences, taking account of variation in the flanking sequence between the specific L. serriola donor and the L. sativa reference genome. Therefore, the SNP marker at nucleotide 101 of SEQ ID NO: 1 to 10 may also be present in a sequence comprising less than 100% sequence identity to the sequence provided under SEQ ID NO: 1 to 10, e.g. at nucleotide 101 of a sequence comprising at least 95%, 96%, 97%, 98%, 99% sequence identity to the provided sequence.

Also, the molecular marker sequences (and isolated nucleic acid molecules comprising the sequence) disclosed herein and their use in detecting and/or generating lettuce plants comprising said QTLs described herein are encompassed herein.

Further a method of growing a plant comprising QTL7.1 (or a variant thereof) in an area where Fusarium races Fol-4 and/or Fol-1 occur, e.g. in Mediterranean countries (e.g. Spain, Italy, France) or in Northern European countries (e.g. Netherlands, Belgium, etc.), either in the field, or in glasshouses or tunnels, is provided. Due to the QTL7.1 being present in the lettuce plants, less yield loss will occur during the cultivation period compared to plants lacking QTL7.1. Growing disease resistant plants is the best way to control effects of Fusarium wilt. There is less of a need for fallowing, sanitation procedures (e.g. removal of debris, cleaning of equipment, etc.), soil fumigation or solarization, crop rotation and/or fungicide treatment when a plant comprising QTL7.1 is grown.

In a further aspect a method for transferring QTL7.1 from a Lactuca serriola plant into a Lactuca sativa plant to generate a Fol-4 and/or Fol-1 resistant cultivated lettuce plant is provided, comprising:
a) crossing a Lactuca serriola plant comprising QTL7.1 with a Lactuca sativa plant to generate an F1;
b) optionally selfing the F1 one or more times to generate further selfing progeny;
c) backcrossing the F1 or further selfing progeny one or more times to the Lactuca sativa plant of step a); and
d) identifying and/or selecting backcross progeny comprising a genome of the Lactuca sativa plant of step a) comprising an introgression fragment of the Lactuca serriola plant of step a) on chromosome 7 comprising QTL7.1,
wherein markers indicative of QTL7.1 are one or more or all of:
   a Guanine (G) for SNP_02 at nucleotide 101 of SEQ ID NO: 2 or at the equivalent nucleotide of a sequence comprising at least 95% sequence identity to SEQ ID NO: 2,
   a Cytosine (C) for SNP_03 at nucleotide 101 of SEQ ID NO: 3 or at the equivalent nucleotide of a sequence comprising at least 95% sequence identity to SEQ ID NO: 3,
   an Adenine (A) for SNP_04 at nucleotide 101 of SEQ ID NO: 4 or at the equivalent nucleotide of a sequence comprising at least 95% sequence identity to SEQ ID NO: 4,
   a Guanine (G) for SNP_05 at nucleotide 101 of SEQ ID NO: 5 or at the equivalent nucleotide of a sequence comprising at least 95% sequence identity to SEQ ID NO: 5,
   a Guanine (G) for SNP_06 at nucleotide 101 of SEQ ID NO: 6 or at the equivalent nucleotide of a sequence comprising at least 95% sequence identity to SEQ ID NO: 6,
   a Thymine (T) for SNP_07 at nucleotide 101 of SEQ ID NO: 7 or at the equivalent nucleotide of a sequence comprising at least 95% sequence identity to SEQ ID NO: 7,
   a Thymine (T) for SNP_08 at nucleotide 101 of SEQ ID NO: 8 or at the equivalent nucleotide of a sequence comprising at least 95% sequence identity to SEQ ID NO: 8,
   an Adenine (A) for SNP_09 at nucleotide 101 of SEQ ID NO: 9 or at the equivalent nucleotide of a sequence comprising at least 95% sequence identity to SEQ ID NO: 9.

In one aspect the L. serriola plant of step a) is a plant of which a representative sample of seeds has been deposited under Accession number NCIMB 44108, or progeny thereof obtained by selfing and/or crossing, wherein the progeny comprise QTL7.1.

In yet a further aspect a method for transferring QTL7.1 from a Lactuca sativa plant into another Lactuca sativa plant to generate a Fol-4 and/or Fol-1 resistant cultivated lettuce plant is provided, comprising:
a) crossing a first Lactuca sativa plant comprising QTL7.1 with another, second Lactuca sativa plant to generate an F1;
b) collecting F1 seeds from said cross and optionally selfing the F1 plants one or more times to generate an F2 or F3 or further selfing population;
c) optionally backcrossing the F1 plant or F2 or F3 plant, or further selfing plant, to the second L. sativa plant of a) to produce a backcross population;
d) optionally selfing the backcross population one or more times; and
e) identifying a F1, F2, F3, further selfing or backcross plant which comprises one or more or all SNP markers indicative of QTL7.1, wherein markers indicative of QTL7.1 are one or more or all of:
   a Guanine (G) for SNP_02 at nucleotide 101 of SEQ ID NO: 2 or at the equivalent nucleotide of a sequence comprising at least 95% sequence identity to SEQ ID NO: 2,
   a Cytosine (C) for SNP_03 at nucleotide 101 of SEQ ID NO: 3 or at the equivalent nucleotide of a sequence comprising at least 95% sequence identity to SEQ ID NO: 3,
   an Adenine (A) for SNP_04 at nucleotide 101 of SEQ ID NO: 4 or at the equivalent nucleotide of a sequence comprising at least 95% sequence identity to SEQ ID NO: 4,
   a Guanine (G) for SNP_05 at nucleotide 101 of SEQ ID NO: 5 or at the equivalent nucleotide of a sequence comprising at least 95% sequence identity to SEQ ID NO: 5,
   a Guanine (G) for SNP_06 at nucleotide 101 of SEQ ID NO: 6 or at the equivalent nucleotide of a sequence comprising at least 95% sequence identity to SEQ ID NO: 6,
   a Thymine (T) for SNP_07 at nucleotide 101 of SEQ ID NO: 7 or at the equivalent nucleotide of a sequence comprising at least 95% sequence identity to SEQ ID NO: 7,
   a Thymine (T) for SNP_08 at nucleotide 101 of SEQ ID NO: 8 or at the equivalent nucleotide of a sequence comprising at least 95% sequence identity to SEQ ID NO: 8,
   an Adenine (A) for SNP_09 at nucleotide 101 of SEQ ID NO: 9 or at the equivalent nucleotide of a sequence comprising at least 95% sequence identity to SEQ ID NO: 9.

Also provided is a method for generating a cultivated lettuce (Lactuca sativa) plant comprising Fol-4 and/or Fol-1 resistance comprising the steps of:
a) providing a cultivated lettuce plant comprising 1, 2, 3, 4, 5, 6, 7, 8, or more SNP markers indicative of QTL7.1;
b) crossing a cultivated lettuce plant with a lettuce plant which is susceptible to Fol-4 and/or Fol-1 or which only comprises intermediate resistance to Fol-4 and/or Fol-1, to produce F1 seeds;
c) optionally selfing the plants grown from F1 seeds one or more times to produce F2, F3 or further generation selfing progeny;
d) identifying F1, F2, F3 or further generation selfing progeny which have Fol-4 and/or Fol-1 resistance and/or which comprise the introgression fragment on chromosome 7 comprising QTL7.1;
e) optionally crossing said identified F1 or further generation selfing progeny to the cultivated lettuce plant of step b) to produce a backcross progeny; and
f) optionally selecting backcross progeny which comprises resistance against Fol-4 and/or Fol-1 and/or which comprises the introgression fragment comprising QTL7.1,
wherein the markers indicative of QTL7.1 in step a) are one or more or all of:
   a Guanine (G) for SNP_02 at nucleotide 101 of SEQ ID NO: 2 or at the equivalent nucleotide of a sequence comprising at least 95% sequence identity to SEQ ID NO: 2,
   a Cytosine (C) for SNP_03 at nucleotide 101 of SEQ ID NO: 3 or at the equivalent nucleotide of a sequence comprising at least 95% sequence identity to SEQ ID NO: 3,
   an Adenine (A) for SNP_04 at nucleotide 101 of SEQ ID NO: 4 or at the equivalent nucleotide of a sequence comprising at least 95% sequence identity to SEQ ID NO: 4,
   a Guanine (G) for SNP_05 at nucleotide 101 of SEQ ID NO: 5 or at the equivalent nucleotide of a sequence comprising at least 95% sequence identity to SEQ ID NO: 5,
   a Guanine (G) for SNP_06 at nucleotide 101 of SEQ ID NO: 6 or at the equivalent nucleotide of a sequence comprising at least 95% sequence identity to SEQ ID NO: 6,
   a Thymine (T) for SNP_07 at nucleotide 101 of SEQ ID NO: 7 or at the equivalent nucleotide of a sequence comprising at least 95% sequence identity to SEQ ID NO: 7,
   a Thymine (T) for SNP_08 at nucleotide 101 of SEQ ID NO: 8 or at the equivalent nucleotide of a sequence comprising at least 95% sequence identity to SEQ ID NO: 8,
   an Adenine (A) for SNP_09 at nucleotide 101 of SEQ ID NO: 9 or at the equivalent nucleotide of a sequence comprising at least 95% sequence identity to SEQ ID NO: 9.

In the above methods the susceptible L. sativa plant or plant having intermediate resistance against Fol-4 and/or Fol-1 is preferably a plant lacking QTL7.1. The plant may comprise some level of resistance against Fusarium, but conferred by a different locus than QTL7.1. Thus, QTL7.1 can be combined with other Fusarium resistance genes/loci found at different locations in the genome.

Further, it is understood that reference to a SNP marker indicative of QTL7.1 refers to the donor SNP haplotype (nucleotides on one chromosome 7 in the plant) and/or the donor SNP genotype (nucleotides on both chromosomes 7 in the plant) of one or more or all of the SNP markers provided herein, linked to QTL7.1, e.g. one or more of SNP_01 to SNP_10, preferably one or more of SNP_02 to SNP 09.

### Deposits

A total of 2500 seeds of Lactuca sativa containing the QTL7.1 from the L. serriola donor in homozygous form were deposited by Nunhems B.V. (Napoleonsweg 152, 6083 AB Nunhem, The Netherlands) under the Budapest Treaty, using the Expert Solution, at the depositor institution NCIMB Limited, Wellheads Place, Dyce, Aberdeen, AB21 7 GB and received accession number NCIMB 44108 on Feb. 1, 2023.

The deposited seeds do not meet the DUS criteria which are required for obtaining plant variety protection, and can therefore not be considered to be a plant variety.

The Applicant requests that samples of the biological material and any material derived therefrom be only released to a designated Expert in accordance with Rule 32 (1) EPC or related legislation of countries or treaties having similar rules and regulation, until the mention of the grant of the patent, or for 20 years from the date of filing if the application is refused, withdrawn or deemed to be withdrawn.

Access to the deposit will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The following non-limiting Examples describe how one can obtain plants comprising QTL7.1 or a variant thereof. Examples provided herein are non-limiting examples. Any patent and non-patents documents mentioned herein are incorporated by reference.

### Sequence Data

SEQ ID NO 1: consensus flanking sequence comprising SNP_01 at nucleotide 101, with SNP nucleotide of resistant donor
SEQ ID NO 2: consensus flanking sequence comprising SNP_02 at nucleotide 101, with SNP nucleotide of resistant donor
SEQ ID NO 3: consensus flanking sequence comprising SNP_03 at nucleotide 101, with SNP nucleotide of resistant donor
SEQ ID NO 4: consensus flanking sequence comprising SNP_04 at nucleotide 101, with SNP nucleotide of resistant donor
SEQ ID NO 5: consensus flanking sequence comprising SNP_05 at nucleotide 101, with SNP nucleotide of resistant donor
SEQ ID NO 6: consensus flanking sequence comprising SNP_06 at nucleotide 101, with SNP nucleotide of resistant donor
SEQ ID NO 7: consensus flanking sequence comprising SNP_07 at nucleotide 101, with SNP nucleotide of resistant donor
SEQ ID NO 8: consensus flanking sequence comprising SNP_08 at nucleotide 101, with SNP nucleotide of resistant donor
SEQ ID NO 9: consensus flanking sequence comprising SNP_09 at nucleotide 101, with SNP nucleotide of resistant donor
SEQ ID NO 10: consensus flanking sequence comprising SNP_10 at nucleotide 101, with SNP nucleotide of resistant donor
SEQ ID NO 11: L. serriola donor flanking sequence comprising SNP_01 at nucleotide 101, with
SNP nucleotide of resistant donor
SEQ ID NO 12: L. serriola donor flanking sequence comprising SNP_02 at nucleotide 101, with SNP nucleotide of resistant donor
SEQ ID NO 13: L. serriola donor flanking sequence comprising SNP_03 at nucleotide 101, with SNP nucleotide of resistant donor
SEQ ID NO 14: L. serriola donor flanking sequence comprising SNP_04 at nucleotide 101, with SNP nucleotide of resistant donor
SEQ ID NO 15: L. serriola donor flanking sequence comprising SNP_05 at nucleotide 101, with SNP nucleotide of resistant donor
SEQ ID NO 16: L. serriola donor flanking sequence comprising SNP_06 at nucleotide 101, with SNP nucleotide of resistant donor
SEQ ID NO 17: L. serriola donor flanking sequence comprising SNP_07 at nucleotide 101, with SNP nucleotide of resistant donor
SEQ ID NO 18: L. serriola donor flanking sequence comprising SNP_08 at nucleotide 101, with SNP nucleotide of resistant donor
SEQ ID NO 19: L. serriola donor flanking sequence comprising SNP_09 at nucleotide 101, with SNP nucleotide of resistant donor
SEQ ID NO 20: L. serriola donor flanking sequence comprising SNP_10 at nucleotide 101, with SNP nucleotide of resistant donor
SEQ ID NO 21: L. sativa reference genome V11 flanking sequence comprising SNP_01 at nucleotide 101, with SNP nucleotide of resistant donor
SEQ ID NO 22: L. sativa reference genome V11 flanking sequence comprising SNP_02 at nucleotide 101, with SNP nucleotide of resistant donor
SEQ ID NO 23: L. sativa reference genome V11 flanking sequence comprising SNP_03 at nucleotide 101, with SNP nucleotide of resistant donor
SEQ ID NO 24: L. sativa reference genome V11 flanking sequence comprising SNP_04 at nucleotide 101, with SNP nucleotide of resistant donor
SEQ ID NO 25: L. sativa reference genome V11 flanking sequence comprising SNP_05 at nucleotide 101, with SNP nucleotide of resistant donor
SEQ ID NO 26: L. sativa reference genome V11 flanking sequence comprising SNP_06 at nucleotide 101, with SNP nucleotide of resistant donor
SEQ ID NO 27: L. sativa reference genome V11 flanking sequence comprising SNP_07 at nucleotide 101, with SNP nucleotide of resistant donor
SEQ ID NO 28: L. sativa reference genome V11 flanking sequence comprising SNP_08 at nucleotide 101, with SNP nucleotide of resistant donor
SEQ ID NO 29: L. sativa reference genome V11 flanking sequence comprising SNP_09 at nucleotide 101, with SNP nucleotide of resistant donor
SEQ ID NO 30: L. sativa reference genome V11 flanking sequence comprising SNP_10 at nucleotide 101, with SNP nucleotide of resistant donor

In the consensus sequences of SEQ ID NO: 1 to SEQ ID NO: 10, differences in the regions flanking the SNP nucleotide are designated following the IUPAC nucleotide ambiguity code, as shown below:

| | |
|---|---|
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

It is noted that the differences between the L. serriola flanking sequences and the L. sativa reference genome V11 flanking sequences are small, with no differences found for sequences flanking SNP 3, SNP_4, SNP_5 and SNP_7; and one nucleotide difference found for sequences flanking SNP_06 and SNP_08; and two nucleotide differences found for sequences flanking SNP_01 and SNP_02; and 5 differences found for sequences flanking SNP_10; and 9 differences found for sequences flanking SNP_09. The most differences are thus found between SEQ ID NO: 09 (SNP_09 with L. serriola flanking sequences) and SEQ ID NO: 19 (SNP_09 with L. sativa reference genome V11 flanking sequences), which sequences still have a sequence identity of 95.5% when aligned pairwise (using Emboss Needle).

### Examples

A large in-house core set of germplasm, consisting of Lactuca sativa, L. serriola, L. saligna and L. virosa species, was screened in a high-throughput in vivo root assay for resistance against Fusarium oxysporum f.sp. lactucae races Fol-1 and Fol-4.

The high throughput screening led to the identification of the L. serriola donor accession, which had been obtained from the CGN collection in the Netherlands and which originated from Georgia.

Through subsequent re-screening in a resistance assay the donor was confirmed to display an intermediary to high level of resistance against the Fol-1 and Fol-4 races of Fusarium oxysporum f. sp. lactucae.

To map the underlying QTLs, a segregating F2 mapping population was created by crossing the donor with an elite susceptible variety. The resulting F2 populations were subjected to QTL mapping (n=184 each for Fol-1 and Fol-4 in separate screenings).

The in-house routine in vivo protocol was followed to generate the phenotypic data and the tissue samples from the corresponding plants were gathered to generate genotypic data on the infinium XT genotypic platform involving 18 k markers. Out of these, around 2000 markers were found to be informative and nicely covering the genome with good distribution for this specific mapping population. By using IM analysis, we identified major QTLs conferring intermediary level of resistance to the races Fol-1 and Fol-4.

The QTL was fine mapped to the region starting at SNP_01 at nucleotide 64.382.881 (SNP_01) of chromosome 7 of the reference genome V11, and ending at SNP_10 at nucleotide 69.375.685 (SNP_10) of chromosome 7 of the reference genome V11. SNP_01 and SNP_10 are, therefore, markers that flank the region comprising QTL7.1. The L. sativa V11 reference genome is found on the world wide web at ncbi.nlm.nih.gov/data-hub/genome/?taxon=4236 and a BLAST can be done with any of the sequences provided herein to identify the nucleotide position of the SNP on chromosome 7 of the V11 reference genome. The genome is also referred to as Lactuca sativa cultivar Salinas, Lsat_Salinas_v11.

The genomic region comprising QTL7.1 from the L. serriola donor is characterized by the presence of QTL7.1 and one or more of the following SNP markers linked to the QTL7.1, as shown in the Tables below.

The Table 1 shows the consensus sequence flanking the SNP, which takes nucleotide differences (in the sequence region flanking the SNP nucleotide) between the wild L. serriola donor (shown in Table 2) and the L. sativa reference genome (shown in Table 3) into account. So, for example, a ‘C’ in the wild donor and an ‘A’ in the L. sativa V11 reference genome is shown as ‘M’ in the consensus sequence. The consensus sequence is based on the IUPAC nucleotide code.

An example of a BLAST output of SEQ ID NO: 1 (Query) against the Lsat_Salinas_v11 is shown in FIG. 3, where the ‘Query’ sequence is aligned to the ‘Sbjct’ chromosome 7 sequence of the L. sativa V11 genome. The SNP at nucleotide 101 of SEQ ID NO: 1 is located at nucleotide 64.382.881 of chromosome 7 of the L. sativa V11 genome. Both sequences have 99% sequence identity in the BLAST output (not shown).

TABLE 1

| | | | | Sequences (consensus flanking |
|---|---|---|---|---|
| | SNP position on | | | sequence of the SNP, |
| | chromosome 7 | SNP | | which is a consensus |
| SNP | of the V11 | nucleotide | SNP | sequence between the |
| marker at | Lactuca sativa | of resistant | nucleotide | resistant donor flanking |
| nucleotide | reference | donor | of | sequence and the |
| 101 of the | genome | (donorS NP | recurrent | L. sativa V11 reference |
| sequence | (nucleotide) | haplotype) | parent | genome flanking sequence) |
| SNP_01 | 64.382.881 | A | G | GTGTAAGAATACAAAATTCTCGCTAC |
| (flanking | | | | TAATAGAATATGTTAGATTCTAACAT |
| marker) | | | | TTTATGCAACAGTCMTTTTMGTAATG |
| | | | | TCATGACACAAAAGTCACCAAG[G/A] |
| | | | | CTCGGCCAGTGAAATTACAAAATACT |
| | | | | CTATTGGAGATTGTCACAATACAATT |
| | | | | CCATAGCCATGAAGTCTCATATTCAA |
| | | | | AGTACATTTCTTTTAACATCCT |
| | | | | (SEQ ID NO: 1) |
| SNP_02 | 64.442.022 | G | A | AAGGACCAGAAGCATTAAAATTGTGT |
| | | | | GTTMGGTTCAGAACKACATCTCTCAC |
| | | | | TAACAAAACCTTCATGACGACCATGT |
| | | | | ATGACTAGACATACACGTACAG[A/G] |
| | | | | CGGACTGAAATTGAAACAGGAAAAT |
| | | | | AGAAATCGCACACACCGAAGCTTCAC |
| | | | | GATCTTGAAATCAACGATTTTGGAGG |
| | | | | CTCCGAGGCTATTTTCATGATTC |
| | | | | (SEQ ID NO: 2) |
| SNP_03 | 64.505.410 | C | T | ACTTTTGGAAAAAACAGATTTGATTC |
| | | | | GTTCCACAAGGGAGTGCCTTATGGTG |
| | | | | TGATGGTTTCTGGAGGTACTGAAGAT |
| | | | | GATGATGAACCAACTTATTGTT[T/C]T |
| | | | | CTATTTGCTGCTTCTTTCTTTAGTTTC |
| | | | | CTTGCACTTCTTTCGATTTCTGGATCA |
| | | | | AAAGCTAAAGGTGTACCTAAATAAA |
| | | | | AAATTATGGGCATAAATGTG |
| | | | | (SEQ ID NO: 3) |
| SNP_04 | 65.772.249 | A | G | GTATTTTTGTCTTTTCAGATTATTCCA |
| | | | | AGAAGAAAGACCTGTTTGGTATACAA |
| | | | | TCAACTGTAACCCACTCCCGAAAGAT |
| | | | | TACAAACAACCAAAAATTGAA[G/A]A |
| | | | | TGACATTACACATGAACAGAAGATCC |
| | | | | TTATGGTAATTGATAAGGAACCTTCA |
| | | | | TCATCATCATCATCATCATCATCAAA |
| | | | | CACTGATTCAGAAGAACACCC |
| | | | | (SEQ ID NO: 4) |
| SNP_05 | 66.347.850 | G | A | CATCAGACAATCCGTTTCTTGAGAAT |
| | | | | TTGTTTCTTAACTTTTGTGAAACACGT |
| | | | | TCGCCTCTTAAGTTCTCTCTACCGAAA |
| | | | | AGCCAAAAGGATCTTGATAT[A/G]GG |
| | | | | TAACCGCCATCATTTGTTGGACTGCA |
| | | | | TGTATCACTTGAAGGTATGCCATGCT |
| | | | | AAACCACCTCCAACCCCTTTGATTCG |
| | | | | AACAGATTACTCCATTGATG |
| | | | | (SEQ ID NO: 5) |
| SNP_06 | 67.607.756 | G | T | GTTGTCCATTATGCTTCCAATGGATGT |
| | | | | ATCCATTTGAAAGATAGATGAAGGTA |
| | | | | ATAAAAGGTCATGTTTGGAACAAAAA |
| | | | | CAAACTGGAAGGATGCATTGT[T/G]K |
| | | | | GAGTGGATGTTGCAGAGGAGACAATT |
| | | | | GAGTTCTTAAGTGAATACCACAAAAC |
| | | | | AAACAGACTGTTGATACTCCACAGGA |
| | | | | TAAGCATAACACAATTGATAA |
| | | | | (SEQ ID NO: 6) |
| SNP_07 | 67.736.843 | T | C | AAAATTTGTAGTTTCGAATTTGCCTTT |
| (peak | | | | TAATGTATCAAGCTCAGCAATTAACT |
| marker) | | | | CAACATCCTGTCTTTTGATATAGTCAT |
| | | | | AGTTTTCACATTTGTTACTG[C/T]AAT |
| | | | | CTTCTTGAAGCCTCCTAAAGTCTTTGA |
| | | | | TTTTTTCTTCTAATTCAGCCTTTAGGG |
| | | | | GTTTCTGTCCTTTCCTGAGTTGATATC |
| | | | | CTTCGTATCTCAGGTC |
| | | | | (SEQ ID NO: 7) |
| SNP_08 | 68.702.173 | T | C | GGCCMATTGGAAACACATTGAAAGT |
| | | | | TGGAGATAGAGTTAATGTTTCTACCA |
| | | | | CTGTGATGAATGGGTTGGAGATACTC |
| | | | | GAGTGTGGTGTAAGCCTTGTGTA[C/T] |
| | | | | ACACATGATGATGATGAGGTAGTAAA |
| | | | | TGAGACCTTGGAAAATGACATGGAAT |
| | | | | GGGTAAAAATTCTTGGTGGAGATTAT |
| | | | | TCTGGATTTCAACTAAGAACAG |
| | | | | (SEQ ID NO: 8) |
| SNP_09 | 69.331.664 | A | G | AAATTGGATATSTTGTGATGTGTGTCC |
| | | | | CTATTTGTGTATGGTATATGTGTTGGA |
| | | | | AATGTTGATGCTCTATTRTGATARAA |
| | | | | TTATTTATGGTGTATTTCAY[G/A]CCA |
| | | | | GAKAGYTTATGTGATGATTTGGTATG |
| | | | | AATWATGATGATAGGTGCATACTAGT |
| | | | | GTGACGACTATTTTTTAWATGYGTTG |
| | | | | TAAATAATGTTTGCATGCT |
| | | | | (SEQ ID NO: 9) |
| SNP_10 | 69.375.685 | G | T | AGAGCATGACTTAGTGAGKTTGGGTT |
| (flanking | | | | CCTTTGGGTAGTTAGTTTTAATAAAG |
| marker) | | | | ATGCCACTTGAGCACGTTTGGTGTTT |
| | | | | ATTTTCCTTTGAACCTCTTTTT[T/G]CA |
| | | | | ATAATTAAATGGTTGAATGCTTGGTT |
| | | | | ATGAAAATGGTAACRTGTGGYTKTGA |
| | | | | AAATGTTTTGAATTAAAAACAAAGTC |
| | | | | TTCCTCTMACGTATTCTGTT |
| | | | | (SEQ ID NO: 10) |

TABLE 2

| | | SNP | | |
|---|---|---|---|---|
| | SNP position | nucleotide | | |
| | on chromosome | of | SNP | |
| | 7 of the V11 | resistant | nucleotide | |
| | reference | donore | of | |
| SNP | genome | (donor SNP | recurrent | Sequences (L. serriola |
| marker | (nucleotide) | haplotype) | parent | flanking sequence) |
| SNP_01 | 64.382.881 | A | G | GTGTAAGAATACAAAATTCTCGCTACT |
| (flanking | | | | AATAGAATATGTTAGATTCTAACATTT |
| marker) | | | | TATGCAACAGTCCTTTTCGTAATGTCA |
| | | | | TGACACAAAAGTCACCAAG[G/A]CTCG |
| | | | | GCCAGTGAAATTACAAAATACTCTATT |
| | | | | GGAGATTGTCACAATACAATTCCATAG |
| | | | | CCATGAAGTCTCATATTCAAAGTACAT |
| | | | | TTCTTTTAACATCCT |
| | | | | (SEQ ID NO: 11) |
| SNP_02 | 64.442.022 | G | A | AAGGACCAGAAGCATTAAAATTGTGT |
| | | | | GTTCGGTTCAGAACTACATCTCTCACT |
| | | | | AACAAAACCTTCATGACGACCATGTAT |
| | | | | GACTAGACATACACGTACAG[A/G]CGG |
| | | | | ACTGAAATTGAAACAGGAAAATAGAA |
| | | | | ATCGCACACACCGAAGCTTCACGATCT |
| | | | | TGAAATCAACGATTTTGGAGGCTCCGA |
| | | | | GGCTATTTTCATGATTC |
| | | | | (SEQ ID NO: 12) |
| SNP_03 | 64.505.410 | C | T | ACTTTTGGAAAAAACAGATTTGATTCG |
| | | | | TTCCACAAGGGAGTGCCTTATGGTGTG |
| | | | | ATGGTTTCTGGAGGTACTGAAGATGAT |
| | | | | GATGAACCAACTTATTGTT[T/C]TCTAT |
| | | | | TTGCTGCTTCTTTCTTTAGTTTCCTTGC |
| | | | | ACTTCTTTCGATTTCTGGATCAAAAGC |
| | | | | TAAAGGTGTACCTAAATAAAAAATTAT |
| | | | | GGGCATAAATGTG |
| | | | | (SEQ ID NO: 13) |
| SNP_04 | 65.772.249 | A | G | GTATTTTTGTCTTTTCAGATTATTCCAA |
| | | | | GAAGAAAGACCTGTTTGGTATACAATC |
| | | | | AACTGTAACCCACTCCCGAAAGATTAC |
| | | | | AAACAACCAAAAATTGAA[G/A]ATGAC |
| | | | | ATTACACATGAACAGAAGATCCTTATG |
| | | | | GTAATTGATAAGGAACCTTCATCATCA |
| | | | | TCATCATCATCATCATCAAACACTGAT |
| | | | | TCAGAAGAACACCC |
| | | | | (SEQ ID NO: 14) |
| SNP_05 | 66.347.850 | G | A | CATCAGACAATCCGTTTCTTGAGAATT |
| | | | | TGTTTCTTAACTTTTGTGAAACACGTTC |
| | | | | GCCTCTTAAGTTCTCTCTACCGAAAAG |
| | | | | CCAAAAGGATCTTGATAT[A/G]GGTAA |
| | | | | CCGCCATCATTTGTTGGACTGCATGTA |
| | | | | TCACTTGAAGGTATGCCATGCTAAACC |
| | | | | ACCTCCAACCCCTTTGATTCGAACAGA |
| | | | | TTACTCCATTGATG |
| | | | | (SEQ ID NO: 15) |
| SNP_06 | 67.607.756 | G | T | GTTGTCCATTATGCTTCCAATGGATGT |
| | | | | ATCCATTTGAAAGATAGATGAAGGTA |
| | | | | ATAAAAGGTCATGTTTGGAACAAAAA |
| | | | | CAAACTGGAAGGATGCATTGT[T/G]TG |
| | | | | AGTGGATGTTGCAGAGGAGACAATTG |
| | | | | AGTTCTTAAGTGAATACCACAAAACAA |
| | | | | ACAGACTGTTGATACTCCACAGGATAA |
| | | | | GCATAACACAATTGATAA |
| | | | | (SEQ ID NO: 16) |
| SNP_07 | 67.736.843 | T | C | AAAATTTGTAGTTTCGAATTTGCCTTTT |
| (peak | | | | AATGTATCAAGCTCAGCAATTAACTCA |
| marker) | | | | ACATCCTGTCTTTTGATATAGTCATAG |
| | | | | TTTTCACATTTGTTACTG[C/T]AATCTTC |
| | | | | TTGAAGCCTCCTAAAGTCTTTGATTTTT |
| | | | | TCTTCTAATTCAGCCTTTAGGGGTTTCT |
| | | | | GTCCTTTCCTGAGTTGATATCCTTCGTA |
| | | | | TCTCAGGTC |
| | | | | (SEQ ID NO: 17) |
| SNP_08 | 68.702.173 | T | C | GGCCCATTGGAAACACATTGAAAGTTG |
| | | | | GAGATAGAGTTAATGTTTCTACCACTG |
| | | | | TGATGAATGGGTTGGAGATACTCGAGT |
| | | | | GTGGTGTAAGCCTTGTGTA[C/T]ACACA |
| | | | | TGATGATGATGAGGTAGTAAATGAGA |
| | | | | CCTTGGAAAATGACATGGAATGGGTA |
| | | | | AAAATTCTTGGTGGAGATTATTCTGGA |
| | | | | TTTCAACTAAGAACAG |
| | | | | (SEQ ID NO: 18) |
| SNP_09 | 69.331.664 | A | G | AAATTGGATATGTTGTGATGTGTGTCC |
| | | | | CTATTTGTGTATGGTATATGTGTTGGA |
| | | | | AATGTTGATGCTCTATTGTGATAGAAT |
| | | | | TATTTATGGTGTATTTCAC[G/A]CCAGA |
| | | | | GAGCTTATGTGATGATTTGGTATGAAT |
| | | | | AATGATGATAGGTGCATACTAGTGTGA |
| | | | | CGACTATTTTTTAAATGCGTTGTAAAT |
| | | | | AATGTTTGCATGCT |
| | | | | (SEQ ID NO: 19) |
| SNP_10 | 69.375.685 | G | T | AGAGCATGACTTAGTGAGGTTGGGTTC |
| (flanking | | | | CTTTGGGTAGTTAGTTTTAATAAAGAT |
| marker) | | | | GCCACTTGAGCACGTTTGGTGTTTATT |
| | | | | TTCCTTTGAACCTCTTTTT[T/G]CAATA |
| | | | | ATTAAATGGTTGAATGCTTGGTTATGA |
| | | | | AAATGGTAACATGTGGCTGTGAAAAT |
| | | | | GTTTTGAATTAAAAACAAAGTCTTCCT |
| | | | | CTAACGTATTCTGTT |
| | | | | (SEQ ID NO: 20) |

TABLE 3

| | SNP | | | |
|---|---|---|---|---|
| | position on | SNP | | |
| | chromosome | nucleotide | SNP | |
| | 7 of the V11 | of resistant | nucleotide | |
| | reference | donor | of | |
| | genome | (donor SNP | recurrent | Sequences (L. sativa reference |
| SNP marker | (nucleotide) | haplotype) | parent | genome flanking sequence) |
| SNP_01 | 64.382.881 | A | G | GTGTAAGAATACAAAATTCTCGCTACT |
| (flanking | | | | AATAGAATATGTTAGATTCTAACATTT |
| marker) | | | | TATGCAACAGTCATTTTAGTAATGTCA |
| | | | | TGACACAAAAGTCACCAAG[G/A]CTCG |
| | | | | GCCAGTGAAATTACAAAATACTCTATT |
| | | | | GGAGATTGTCACAATACAATTCCATAG |
| | | | | CCATGAAGTCTCATATTCAAAGTACAT |
| | | | | TTCTTTTAACATCCT |
| | | | | (SEQ ID NO: 21) |
| SNP_02 | 64.442.022 | G | A | AAGGACCAGAAGCATTAAAATTGTGT |
| | | | | GTTAGGTTCAGAACGACATCTCTCACT |
| | | | | AACAAAACCTTCATGACGACCATGTAT |
| | | | | GACTAGACATACACGTACAG[A/G]CGG |
| | | | | ACTGAAATTGAAACAGGAAAATAGAA |
| | | | | ATCGCACACACCGAAGCTTCACGATCT |
| | | | | TGAAATCAACGATTTTGGAGGCTCCGA |
| | | | | GGCTATTTTCATGATTC |
| | | | | (SEQ ID NO: 22) |
| SNP_03 | 64.505.410 | C | T | ACTTTTGGAAAAAACAGATTTGATTCG |
| | | | | TTCCACAAGGGAGTGCCTTATGGTGTG |
| | | | | ATGGTTTCTGGAGGTACTGAAGATGAT |
| | | | | GATGAACCAACTTATTGTT[T/C]TCTAT |
| | | | | TTGCTGCTTCTTTCTTTAGTTTCCTTGC |
| | | | | ACTTCTTTCGATTTCTGGATCAAAAGC |
| | | | | TAAAGGTGTACCTAAATAAAAAATTAT |
| | | | | GGGCATAAATGTG |
| | | | | (SEQ ID NO: 23) |
| SNP_04 | 65.772.249 | A | G | GTATTTTTGTCTTTTCAGATTATTCCAA |
| | | | | GAAGAAAGACCTGTTTGGTATACAATC |
| | | | | AACTGTAACCCACTCCCGAAAGATTAC |
| | | | | AAACAACCAAAAATTGAA[G/A]ATGAC |
| | | | | ATTACACATGAACAGAAGATCCTTATG |
| | | | | GTAATTGATAAGGAACCTTCATCATCA |
| | | | | TCATCATCATCATCATCAAACACTGAT |
| | | | | TCAGAAGAACACCC |
| | | | | (SEQ ID NO: 24) |
| SNP_05 | 66.347.850 | G | A | CATCAGACAATCCGTTTCTTGAGAATT |
| | | | | TGTTTCTTAACTTTTGTGAAACACGTTC |
| | | | | GCCTCTTAAGTTCTCTCTACCGAAAAG |
| | | | | CCAAAAGGATCTTGATAT[A/G]GGTAA |
| | | | | CCGCCATCATTTGTTGGACTGCATGTA |
| | | | | TCACTTGAAGGTATGCCATGCTAAACC |
| | | | | ACCTCCAACCCCTTTGATTCGAACAGA |
| | | | | TTACTCCATTGATG |
| | | | | (SEQ ID NO: 25) |
| SNP_06 | 67.607.756 | G | T | GTTGTCCATTATGCTTCCAATGGATGT |
| | | | | ATCCATTTGAAAGATAGATGAAGGTA |
| | | | | ATAAAAGGTCATGTTTGGAACAAAAA |
| | | | | CAAACTGGAAGGATGCATTGT[T/G]GG |
| | | | | AGTGGATGTTGCAGAGGAGACAATTG |
| | | | | AGTTCTTAAGTGAATACCACAAAACAA |
| | | | | ACAGACTGTTGATACTCCACAGGATAA |
| | | | | GCATAACACAATTGATAA |
| | | | | (SEQ ID NO: 26) |
| SNP_07 | 67.736.843 | T | C | AAAATTTGTAGTTTCGAATTTGCCTTTT |
| (peak | | | | AATGTATCAAGCTCAGCAATTAACTCA |
| marker) | | | | ACATCCTGTCTTTTGATATAGTCATAG |
| | | | | TTTTCACATTTGTTACTG[C/T]AATCTTC |
| | | | | TTGAAGCCTCCTAAAGTCTTTGATTTTT |
| | | | | TCTTCTAATTCAGCCTTTAGGGGTTTCT |
| | | | | GTCCTTTCCTGAGTTGATATCCTTCGTA |
| | | | | TCTCAGGTC |
| | | | | (SEQ ID NO: 27) |
| SNP_08 | 68.702.173 | T | C | GGCCAATTGGAAACACATTGAAAGTT |
| | | | | GGAGATAGAGTTAATGTTTCTACCACT |
| | | | | GTGATGAATGGGTTGGAGATACTCGA |
| | | | | GTGTGGTGTAAGCCTTGTGTA[C/T]ACA |
| | | | | CATGATGATGATGAGGTAGTAAATGA |
| | | | | GACCTTGGAAAATGACATGGAATGGG |
| | | | | TAAAAATTCTTGGTGGAGATTATTCTG |
| | | | | GATTTCAACTAAGAACAG |
| | | | | (SEQ ID NO: 28) |
| SNP_09 | 69.331.664 | A | G | AAATTGGATATCTTGTGATGTGTGTCC |
| | | | | CTATTTGTGTATGGTATATGTGTTGGA |
| | | | | AATGTTGATGCTCTATTATGATAAAAT |
| | | | | TATTTATGGTGTATTTCAT[G/A]CCAGA |
| | | | | TAGTTTATGTGATGATTTGGTATGAAT |
| | | | | TATGATGATAGGTGCATACTAGTGTGA |
| | | | | CGACTATTTTTTATATGTGTTGTAAATA |
| | | | | ATGTTTGCATGCT |
| | | | | (SEQ ID NO: 29) |
| SNP_10 | 69.375.685 | G | T | AGAGCATGACTTAGTGAGTTTGGGTTC |
| (flanking | | | | CTTTGGGTAGTTAGTTTTAATAAAGAT |
| marker) | | | | GCCACTTGAGCACGTTTGGTGTTTATT |
| | | | | TTCCTTTGAACCTCTTTTT[T/G]CAATA |
| | | | | ATTAAATGGTTGAATGCTTGGTTATGA |
| | | | | AAATGGTAACGTGTGGTTTTGAAAATG |
| | | | | TTTTGAATTAAAAACAAAGTCTTCCTC |
| | | | | TCACGTATTCTGTT |
| | | | | (SEQ ID NO: 30) |

The resistant donor nucleotide for SNP markers SNP_02, SNP_03, SNP_04, SNP_05, SNP_06, SNP_08 and SNP_09 appears to be unique to the specific wild L. serriola accession used to map and introgress the QTL7.1.

From the mapping population a L. sativa line was selected which was homozygous for the introgression fragment comprising QTL7.1. Seeds produced by that line were deposited by Nunhems B.V. under the Budapest Treaty and received Accession number NCIMB 44108. The L. serriola donor and the L. sativa line comprising the QTL7.1 introgression (seed deposit NCIMB 44108) were further tested together with the differential set of the ISF (see world wide web at worldseed. org/our-work/disease-resistance/differential-hosts/) in a root dip assay, as described in the CPVO protocol (on the world wide web at cpvo.europa.eu, pages 34 to 36).

In short, 9 plants per genotype were tested with either Fol-1 (reference isolate of the Naktuinbouw) or Fol-4 (isolate 04750888 of Gilardi et al. 2017).

Prior to transplanting, the roots of the young plants were hung into a spore suspension of 10⁶ spores/ml for 5 to 15 minutes.

Phenotypic observations were carried out 18 days after inoculation, using the CPVO scale for ‘inoculation by soaking seedlings’ (see page 35 of the CPVO protocol, with pictures):
0: plant without symptoms and healthy vessels
1: plant with brown vessels only below the cotyledon, without yellowing and wilting
2: plant with brown vessels above the cotyledon, without yellowing and wilting
3: plant yellowing and wilting, brown vessels
4: dead plant

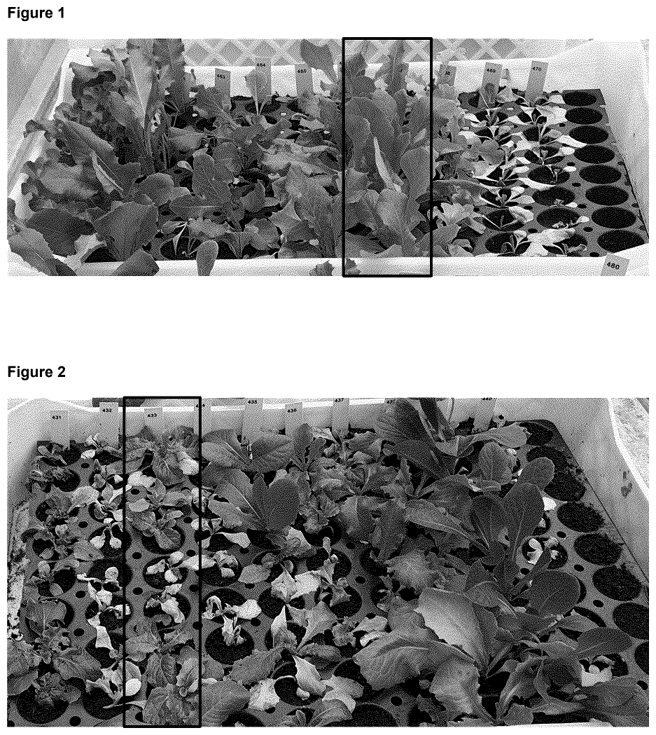

At 12 days after inoculation the fotos of FIG. 1 and FIG. 2 were taken.

The results are shown in Table 4 below:

| | Race Fol-1 | | Race Fol-4 | |
|---|---|---|---|---|
| | | Average | | Average |
| | Classi- | phenotypic | Classi- | phenotypic |
| | fication | score | fication | score |
| L. serriola donor | HR | 0.7 | HR | 1.0 |
| L. sativa line | IR | 2.1 | HR | 0.9 |
| comprising QTL7.1 | | | | |
| (seed deposit | | | | |
| NCIMB 44108) | | | | |
| Recurrent parent | S | 3.6 | S | 3.1 |
| variety | | | | |
| | Classi- | Average | Classi- | Average |
| ISF Differential Set | fication | score | fication | score |
| Gisela | S | 3.8 | S | 3.1 |
| Patriot | S | 3.6 | IR | 1.8 |
| Costa Rica No 4 | HR | 1.0 | S | 3.1 |
| Romabella | HR | 0.0 | IR | 2.5 |
| Banchu Red Fire | S | 2.9 | IR | 2.4 |
| Ballerina | S | 3.3 | IR | 1.9 |
| Lomeria | S | 2.4 | HR | 0.7 |

HR = High Resistance,
IR = Intermediate Resistance,
S = Susceptible

1. A lettuce plant (Lactuca sativa L.) comprising a Quantitative Trait Locus (QTL) named QTL7.1 from Lactuca serriola, which confers resistance to Fusarium oxysporum f.sp. lactucae races Fol-1 and Fol-4, wherein the QTL7.1 is located on chromosome 7 between nucleotide 64.382.881 (SNP_01 at nucleotide 101 of SEQ ID NO: 1) and nucleotide 69.375.685 (SNP_10 at nucleotide 101 of SEQ ID NO: 10) and wherein said QTL7.1 is as found in the genome of plants grown from seeds of which a representative sample was deposited under accession number NCIMB 44108.

2. The lettuce plant according to claim 1, wherein the plant comprises a Thymine (T) for SNP_07 at nucleotide 101 of SEQ ID NO: 7 or at the equivalent nucleotide of a sequence comprising at least 95% sequence identity to SEQ ID NO: 7.

3. The lettuce plant (Lactuca sativa L.) according to claim 1, wherein the plant comprises in its genome at least two, three, four, five, six, seven or eight markers selected from:
a Guanine (G) for SNP_02 at nucleotide 101 of SEQ ID NO: 2 or at the equivalent nucleotide of a sequence comprising at least 95% sequence identity to SEQ ID NO: 2,
a Cytosine (C) for SNP_03 at nucleotide 101 of SEQ ID NO: 3 or at the equivalent nucleotide of a sequence comprising at least 95% sequence identity to SEQ ID NO: 3,
an Adenine (A) for SNP_04 at nucleotide 101 of SEQ ID NO: 4 or at the equivalent nucleotide of a sequence comprising at least 95% sequence identity to SEQ ID NO: 4,
a Guanine (G) for SNP_05 at nucleotide 101 of SEQ ID NO: 5 or at the equivalent nucleotide of a sequence comprising at least 95% sequence identity to SEQ ID NO: 5,
a Guanine (G) for SNP_06 at nucleotide 101 of SEQ ID NO: 6 or at the equivalent nucleotide of a sequence comprising at least 95% sequence identity to SEQ ID NO: 6,
a Thymine (T) for SNP_07 at nucleotide 101 of SEQ ID NO: 7 or at the equivalent nucleotide of a sequence comprising at least 95% sequence identity to SEQ ID NO: 7,
a Thymine (T) for SNP_08 at nucleotide 101 of SEQ ID NO: 8 or at the equivalent nucleotide of a sequence comprising at least 95% sequence identity to SEQ ID NO: 8, and/or
an Adenine (A) for SNP_09 at nucleotide 101 of SEQ ID NO: 9 or at the equivalent nucleotide of a sequence comprising at least 95% sequence identity to SEQ ID NO: 9.

4. The lettuce plant (Lactuca sativa L.) according to claim 1, wherein the plant comprises the QTL7.1 in homozygous form.

5. A seed capable of growing into a lettuce plant as claimed in claim 1.

6. A seed produced on a lettuce plant as claimed in claim 1, wherein said seed comprises QTL7.1.

7. A progeny plant of a lettuce plant as defined in claim 1, wherein the progeny plant comprises QTL7.1.

8. Propagation material derived from a plant as claimed in claim 1, wherein the propagation material comprises the QTL7.1.

9. Propagation material capable of growing into a plant as claimed in claim 1.

10. A method for detecting or selecting a plant or plant part or seed comprising QTL7.1, said method comprising determining the presence in the genome of the plant, plant part or seed of at least 2, 3, 4, 5, 6, 7 or 8 of the following SNP markers:
a Guanine (G) for SNP_02 at nucleotide 101 of SEQ ID NO: 2 or at the equivalent nucleotide of a sequence comprising at least 95% sequence identity to SEQ ID NO: 2,
a Cytosine (C) for SNP_03 at nucleotide 101 of SEQ ID NO: 3 or at the equivalent nucleotide of a sequence comprising at least 95% sequence identity to SEQ ID NO: 3,
an Adenine (A) for SNP_04 at nucleotide 101 of SEQ ID NO: 4 or at the equivalent nucleotide of a sequence comprising at least 95% sequence identity to SEQ ID NO: 4,
a Guanine (G) for SNP_05 at nucleotide 101 of SEQ ID NO: 5 or at the equivalent nucleotide of a sequence comprising at least 95% sequence identity to SEQ ID NO: 5,
a Guanine (G) for SNP_06 at nucleotide 101 of SEQ ID NO: 6 or at the equivalent nucleotide of a sequence comprising at least 95% sequence identity to SEQ ID NO: 6,
a Thymine (T) for SNP_07 at nucleotide 101 of SEQ ID NO: 7 or at the equivalent nucleotide of a sequence comprising at least 95% sequence identity to SEQ ID NO: 7,
a Thymine (T) for SNP_08 at nucleotide 101 of SEQ ID NO: 8 or at the equivalent nucleotide of a sequence comprising at least 95% sequence identity to SEQ ID NO: 8, and/or
an Adenine (A) for SNP_09 at nucleotide 101 of SEQ ID NO: 9 or at the equivalent nucleotide of a sequence comprising at least 95% sequence identity to SEQ ID NO: 9,
and optionally selecting a plant, or plant part or seed comprising said SNP markers.

11. The method according to claim 10, further comprising the step of:
testing the plant or seed comprising said at least 2, 3, 4, 5, 6, 7 or 8 SNP markers for resistance against Fusarium race Fol-4 and/or Fol-1, and optionally selecting the plant or seed comprising resistance against Fusarium race Fol-4 and/or Fol-1.

12. A method of producing an L. sativa plant comprising an introgression fragment on chromosome 7 comprising QTL7.1 comprising: crossing a first lettuce (L. sativa) plant comprising a recombinant chromosome 7 in homozygous form having an introgression fragment comprising QTL7.1 and comprising the donor SNP nucleotide for at least 1, 2, 3, 4, 5, 6, 7, 8, 9 or 10 of the SNP markers linked to the QTL, with a second lettuce (L. sativa) plant.


SEQUENCE LISTING (30 sequences)

SEQ ID NO: 1 - DNA, 201 bp, Lactuca sativa
  source 1..201: mol_type = genomic DNA
  variation 101: note = SNP at nucleotide 101
gtgtaagaatacaaaattctcgctactaatagaatatgttagattctaacattttatgcaacagtcmttttmgtaatgtcatgacacaaaagtcaccaagactcggccagtgaaattacaaaatactctattggagattgtcacaatacaattccatagccatgaagtctcatattcaaagtacatttcttttaacatcct

SEQ ID NO: 2 - DNA, 201 bp, Lactuca sativa
  source 1..201: mol_type = genomic DNA
  variation 101: note = SNP at nucleotide 101
aaggaccagaagcattaaaattgtgtgttmggttcagaackacatctctcactaacaaaaccttcatgacgaccatgtatgactagacatacacgtacaggcggactgaaattgaaacaggaaaatagaaatcgcacacaccgaagcttcacgatcttgaaatcaacgattttggaggctccgaggctattttcatgattc

SEQ ID NO: 3 - DNA, 201 bp, Lactuca sativa
  source 1..201: mol_type = genomic DNA
  variation 101: note = SNP at nucleotide 101
acttttggaaaaaacagatttgattcgttccacaagggagtgccttatggtgtgatggtttctggaggtactgaagatgatgatgaaccaacttattgttctctatttgctgcttctttctttagtttccttgcacttctttcgatttctggatcaaaagctaaaggtgtacctaaataaaaaattatgggcataaatgtg

SEQ ID NO: 4 - DNA, 201 bp, Lactuca sativa
  source 1..201: mol_type = genomic DNA
  variation 101: note = SNP at nucleotide 101
gtatttttgtcttttcagattattccaagaagaaagacctgtttggtatacaatcaactgtaacccactcccgaaagattacaaacaaccaaaaattgaaaatgacattacacatgaacagaagatccttatggtaattgataaggaaccttcatcatcatcatcatcatcatcatcaaacactgattcagaagaacaccc

SEQ ID NO: 5 - DNA, 201 bp, Lactuca sativa
  source 1..201: mol_type = genomic DNA
  variation 101: note = SNP at nucleotide 101
catcagacaatccgtttcttgagaatttgtttcttaacttttgtgaaacacgttcgcctcttaagttctctctaccgaaaagccaaaaggatcttgatatgggtaaccgccatcatttgttggactgcatgtatcacttgaaggtatgccatgctaaaccacctccaacccctttgattcgaacagattactccattgatg

SEQ ID NO: 6 - DNA, 201 bp, Lactuca sativa
  source 1..201: mol_type = genomic DNA
  variation 101: note = SNP at nucleotide 101
gttgtccattatgcttccaatggatgtatccatttgaaagatagatgaaggtaataaaaggtcatgtttggaacaaaaacaaactggaaggatgcattgtgkgagtggatgttgcagaggagacaattgagttcttaagtgaataccacaaaacaaacagactgttgatactccacaggataagcataacacaattgataa

SEQ ID NO: 7 - DNA, 201 bp, Lactuca sativa
  source 1..201: mol_type = genomic DNA
  variation 101: note = SNP at nucleotide 101
aaaatttgtagtttcgaatttgccttttaatgtatcaagctcagcaattaactcaacatcctgtcttttgatatagtcatagttttcacatttgttactgtaatcttcttgaagcctcctaaagtctttgattttttcttctaattcagcctttaggggtttctgtcctttcctgagttgatatccttcgtatctcaggtc

SEQ ID NO: 8 - DNA, 201 bp, Lactuca sativa
  source 1..201: mol_type = genomic DNA
  variation 101: note = SNP at nucleotide 101
ggccmattggaaacacattgaaagttggagatagagttaatgtttctaccactgtgatgaatgggttggagatactcgagtgtggtgtaagccttgtgtatacacatgatgatgatgaggtagtaaatgagaccttggaaaatgacatggaatgggtaaaaattcttggtggagattattctggatttcaactaagaacag

SEQ ID NO: 9 - DNA, 201 bp, Lactuca sativa
  source 1..201: mol_type = genomic DNA
  variation 101: note = SNP at nucleotide 101
aaattggatatsttgtgatgtgtgtccctatttgtgtatggtatatgtgttggaaatgttgatgctctattrtgataraattatttatggtgtatttcayaccagakagyttatgtgatgatttggtatgaatwatgatgataggtgcatactagtgtgacgactattttttawatgygttgtaaataatgtttgcatgct

SEQ ID NO: 10 - DNA, 201 bp, Lactuca sativa
  source 1..201: mol_type = genomic DNA
  variation 101: note = SNP at nucleotide 101
agagcatgacttagtgagkttgggttcctttgggtagttagttttaataaagatgccacttgagcacgtttggtgtttattttcctttgaacctctttttgcaataattaaatggttgaatgcttggttatgaaaatggtaacrtgtggytktgaaaatgttttgaattaaaaacaaagtcttcctctmacgtattctgtt

SEQ ID NO: 11 - DNA, 201 bp, Lactuca serriola
  source 1..201: mol_type = genomic DNA
  variation 101: note = SNP at nucleotide 101
gtgtaagaatacaaaattctcgctactaatagaatatgttagattctaacattttatgcaacagtccttttcgtaatgtcatgacacaaaagtcaccaagactcggccagtgaaattacaaaatactctattggagattgtcacaatacaattccatagccatgaagtctcatattcaaagtacatttcttttaacatcct

SEQ ID NO: 12 - DNA, 201 bp, Lactuca serriola
  source 1..201: mol_type = genomic DNA
  variation 101: note = SNP at nucleotide 101
aaggaccagaagcattaaaattgtgtgttcggttcagaactacatctctcactaacaaaaccttcatgacgaccatgtatgactagacatacacgtacaggcggactgaaattgaaacaggaaaatagaaatcgcacacaccgaagcttcacgatcttgaaatcaacgattttggaggctccgaggctattttcatgattc

SEQ ID NO: 13 - DNA, 201 bp, Lactuca serriola; identical to SEQ ID NO: 3
  source 1..201: mol_type = genomic DNA
  variation 101: note = SNP at nucleotide 101

SEQ ID NO: 14 - DNA, 201 bp, Lactuca serriola; identical to SEQ ID NO: 4
  source 1..201: mol_type = genomic DNA
  variation 101: note = SNP at nucleotide 101

SEQ ID NO: 15 - DNA, 201 bp, Lactuca serriola; identical to SEQ ID NO: 5
  source 1..201: mol_type = genomic DNA
  variation 101: note = SNP at nucleotide 101

SEQ ID NO: 16 - DNA, 201 bp, Lactuca serriola
  source 1..201: mol_type = genomic DNA
  variation 101: note = SNP at nucleotide 101
gttgtccattatgcttccaatggatgtatccatttgaaagatagatgaaggtaataaaaggtcatgtttggaacaaaaacaaactggaaggatgcattgtgtgagtggatgttgcagaggagacaattgagttcttaagtgaataccacaaaacaaacagactgttgatactccacaggataagcataacacaattgataa

SEQ ID NO: 17 - DNA, 201 bp, Lactuca serriola; identical to SEQ ID NO: 7
  source 1..201: mol_type = genomic DNA
  variation 101: note = SNP at nucleotide 101

SEQ ID NO: 18 - DNA, 201 bp, Lactuca serriola
  source 1..201: mol_type = genomic DNA
  variation 101: note = SNP at nucleotide 101
ggcccattggaaacacattgaaagttggagatagagttaatgtttctaccactgtgatgaatgggttggagatactcgagtgtggtgtaagccttgtgtatacacatgatgatgatgaggtagtaaatgagaccttggaaaatgacatggaatgggtaaaaattcttggtggagattattctggatttcaactaagaacag

SEQ ID NO: 19 - DNA, 201 bp, Lactuca serriola
  source 1..201: mol_type = genomic DNA
  variation 101: note = SNP at nucleotide 101
aaattggatatgttgtgatgtgtgtccctatttgtgtatggtatatgtgttggaaatgttgatgctctattgtgatagaattatttatggtgtatttcacaccagagagcttatgtgatgatttggtatgaataatgatgataggtgcatactagtgtgacgactattttttaaatgcgttgtaaataatgtttgcatgct

SEQ ID NO: 20 - DNA, 201 bp, Lactuca serriola
  source 1..201: mol_type = genomic DNA
  variation 101: note = SNP at nucleotide 101
agagcatgacttagtgaggttgggttcctttgggtagttagttttaataaagatgccacttgagcacgtttggtgtttattttcctttgaacctctttttgcaataattaaatggttgaatgcttggttatgaaaatggtaacatgtggctgtgaaaatgttttgaattaaaaacaaagtcttcctctaacgtattctgtt

SEQ ID NO: 21 - DNA, 201 bp, Lactuca sativa
  source 1..201: mol_type = genomic DNA
  variation 101: note = SNP at nucleotide 101
gtgtaagaatacaaaattctcgctactaatagaatatgttagattctaacattttatgcaacagtcattttagtaatgtcatgacacaaaagtcaccaagactcggccagtgaaattacaaaatactctattggagattgtcacaatacaattccatagccatgaagtctcatattcaaagtacatttcttttaacatcct

SEQ ID NO: 22 - DNA, 201 bp, Lactuca sativa
  source 1..201: mol_type = genomic DNA
  variation 101: note = SNP at nucleotide 101
aaggaccagaagcattaaaattgtgtgttaggttcagaacgacatctctcactaacaaaaccttcatgacgaccatgtatgactagacatacacgtacaggcggactgaaattgaaacaggaaaatagaaatcgcacacaccgaagcttcacgatcttgaaatcaacgattttggaggctccgaggctattttcatgattc

SEQ ID NO: 23 - DNA, 201 bp, Lactuca sativa; identical to SEQ ID NO: 3
  source 1..201: mol_type = genomic DNA
  variation 101: note = SNP at nucleotide 101

SEQ ID NO: 24 - DNA, 201 bp, Lactuca sativa; identical to SEQ ID NO: 4
  source 1..201: mol_type = genomic DNA
  variation 101: note = SNP at nucleotide 101

SEQ ID NO: 25 - DNA, 201 bp, Lactuca sativa; identical to SEQ ID NO: 5
  source 1..201: mol_type = genomic DNA
  variation 101: note = SNP at nucleotide 101

SEQ ID NO: 26 - DNA, 201 bp, Lactuca sativa
  source 1..201: mol_type = genomic DNA
  variation 101: note = SNP at nucleotide 101
gttgtccattatgcttccaatggatgtatccatttgaaagatagatgaaggtaataaaaggtcatgtttggaacaaaaacaaactggaaggatgcattgtgggagtggatgttgcagaggagacaattgagttcttaagtgaataccacaaaacaaacagactgttgatactccacaggataagcataacacaattgataa

SEQ ID NO: 27 - DNA, 201 bp, Lactuca sativa; identical to SEQ ID NO: 7
  source 1..201: mol_type = genomic DNA
  variation 101: note = SNP at nucleotide 101

SEQ ID NO: 28 - DNA, 201 bp, Lactuca sativa
  source 1..201: mol_type = genomic DNA
  variation 101: note = SNP at nucleotide 101
ggccaattggaaacacattgaaagttggagatagagttaatgtttctaccactgtgatgaatgggttggagatactcgagtgtggtgtaagccttgtgtatacacatgatgatgatgaggtagtaaatgagaccttggaaaatgacatggaatgggtaaaaattcttggtggagattattctggatttcaactaagaacag

SEQ ID NO: 29 - DNA, 201 bp, Lactuca sativa
  source 1..201: mol_type = genomic DNA
  variation 101: note = SNP at nucleotide 101
aaattggatatcttgtgatgtgtgtccctatttgtgtatggtatatgtgttggaaatgttgatgctctattatgataaaattatttatggtgtatttcataccagatagtttatgtgatgatttggtatgaattatgatgataggtgcatactagtgtgacgactattttttatatgtgttgtaaataatgtttgcatgct

SEQ ID NO: 30 - DNA, 201 bp, Lactuca sativa
  source 1..201: mol_type = genomic DNA
  variation 101: note = SNP at nucleotide 101
agagcatgacttagtgagtttgggttcctttgggtagttagttttaataaagatgccacttgagcacgtttggtgtttattttcctttgaacctctttttgcaataattaaatggttgaatgcttggttatgaaaatggtaacgtgtggttttgaaaatgttttgaattaaaaacaaagtcttcctctcacgtattctgtt